(19) United States
(12) Patent Application Publication
(10) Pub. No.: US 2022/0041669 A1
(43) Pub. Date: Feb. 10, 2022

(54) METHODS OF TREATING OR PREVENTING CONDITIONS OF DENDRITIC AND NEURAL SPINE DEFECTS

(71) Applicant: THE JOHNS HOPKINS UNIVERSITY, Baltimore, MD (US)

(72) Inventors: Sean James Miller, Baltimore, MD (US); Jeffrey D. Rothstein, Catonsville, MD (US)

(21) Appl. No.: 17/277,676

(22) PCT Filed: Sep. 18, 2019
(86) PCT No.: PCT/US2019/051692

§ 371 Date: Mar. 18, 2021

Related U.S. Application Data
(60) Provisional application No. 62/732,628, filed on Sep. 18, 2018.

(51) Int. Cl. C07K 14/475; A61P 25/28

(52) U.S. Cl. CPC C07K 14/475; A61K 38/00; A61P 25/28

(57) ABSTRACT

Described are methods of treating or preventing dendritic spine loss in a subject. The methods include a step of administering to a subject having or at risk of having dendritic spine loss a pharmaceutical composition. In addition, pharmaceutical compositions are described comprises an agent selected from the group comprising a salt, solvate, or stereoisomer of a norrin protein or functional part thereof; a vector expressing the norrin protein or functional part thereof; or a combination thereof, and a pharmaceutically acceptable carrier.

### REFERENCE TO RELATED APPLICATIONS

This application claims the benefit of U.S. Provisional Patent application 62/732,628, filed Sep. 18, 2018, which is hereby incorporated by reference for all purposes as if fully set forth herein.

### STATEMENT OF GOVERNMENTAL INTEREST

This invention was made with government support under grant no. NS092067 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Aug. 22, 2019, is named *.TXT and is * bytes in size.

### BACKGROUND OF THE INVENTION

Astroglia are the most abundant cell type in the central nervous system (CNS) and have essential roles in the development and homeostasis of the nervous system, maturation and maintenance of synapses, and regulation of neural transmission. Disturbances to these essential roles contribute to various CNS diseases, including amyotrophic lateral sclerosis (ALS). For more than 100 years, astroglia have been broadly defined into two morphologically described subgroups: protoplasmic astroglia, which are localized to gray matter, and fibrous astroglia, which are localized to white matter. However, recent years have witnessed a growing appreciation for potential astroglia diversity beyond simple morphology, with accumulating evidence suggesting the existence of functionally distinct astroglia subpopulations. Current knowledge surrounding functional specialization of astroglia subpopulations comes in part from insight provided by the positional identity of astroglia in the developing spinal cord, where defined anatomical locations also define different astroglia subpopulations. Each of these subpopulations displays a unique biological profile that allows the cell to maintain its physiological niche. In disease, altered function in regional astroglia can exacerbate pathogenesis, such as impaired glutamate uptake by perisynaptic astroglia in the context of neurodegeneration. However, very little is known about the molecular identities of different astroglia subgroups in the majority of CNS tissues and how these subgroups might regulate neuronal function. This limited understanding of astroglia is due in part to the lack of RNA or protein markers to identify different subgroups in the adult CNS, making histological or functional studies of different populations nearly impossible.

### SUMMARY OF THE INVENTION

One embodiment of the present invention is a method of treating or preventing dendritic spine loss in a subject. The method includes a step of administering to a subject having or at risk of having dendritic spine loss a pharmaceutical composition. The pharmaceutical composition comprises an agent selected from the group comprising a salt, solvate, or stereoisomer of a norrin protein or functional part thereof; a vector expressing the norrin protein or functional part thereof; or a combination thereof, and a pharmaceutically acceptable carrier. Then treating or preventing the dendritic spine loss in the subject. Examples of suitable vectors used in the present invention includes nanoparticles; retrovirsuses, such as Alpharetrovirus, Betaretrovirus, Deltaretrovirus, Epsilonretrovirus, Gammaretrovirus, and Lentivirus; adenoviruses, such as Atadenovirus, Aviadenovirus, Ichtadenovirus, Mastadenovirus, and Siadenovirus; or a combination thereof. The methods of the present invention are able to treat or prevent diseases, such as amyotrophic lateral sclerosis (ALS) that result in dendritic spine loss in a subject. Subjects having ALS or at risk for ALS may be treated with the methods of the present invention. Norrin protein used in the present invention may be an entire norrin protein or functional part thereof, as examples. Suitable norrin proteins may also be selected from SEQ ID NOs: 1,4,6,7 or a combination thereof, as examples. In some embodiments of the present invention a vector may be administered to a subject. The vector expresses a norrin protein or functional part thereof. Examples of vector sequences expressing a norrin protein include SEQ ID NOs: 2, 3, 5, or a combination thereof.

Another embodiment of the present invention is a method of treating or preventing dendritic spine loss in a subject by administering to a subject a pharmaceutical composition. The pharmaceutical composition comprises an agonist of LGR6 and a pharmaceutically acceptable carrier. Then treating or preventing dendritic spine loss in the subject. An example of an LGR6 agonist includes RSPO1, or a functional part thereof. Examples of RSPO1 protein sequences includes SEQ ID NOs: 15, 16, or a combination thereof. In some embodiments of the present invention the agonist of LGR6 may be a vector that expresses the agonist of LGR6. For example the vector may express the LGR6 agonist RSPO1, or a functional part thereof. Example of nucleic acid sequences that may be placed in vectors of the present invention that express RSPO1, or a functional part thereof, include SEQ ID NOs: 8, 9, 10, 11, 12 or a combination thereof. An additional step may include administering to the subject a pharmaceutical composition comprising a salt, solvate, or steriosomer of a norrin protein or functional part thereof, and a pharmaceutically acceptable carrier. A vector may also be administered that expresses the norrin protein, or functional part thereof. The methods of the present invention may be used to treat and prevent dendritic spine loss in a subject caused by a disease such as amyotrophic lateral sclerosis (ALS).

Another embodiment of the present invention is a method of treating or preventing a condition of dendritic spine loss in a subject comprising the steps of administering to a subject having or prone to a condition of dendritic spine loss a pharmaceutical composition. The pharmaceutical composition comprises a modulator of the expression of Norrin and a pharmaceutically acceptable carrier. Then treating or preventing the condition of dendritic spine loss. An example of a suitable modulator of expression of Norrin is RSPO1, or functional part thereof, or an agonist of LGR6. Examples of RSPO1 proteins include SEQ ID NOs: 15, 16, or a combination thereof. Methods of the present invention may include a vector that expresses an agonist of LGR6. Examples of nucleic acid sequences that may be part of a vector, that express an agonist of LGR6 include SEQ ID NOs: 8, 9, 10, 11, 12 or a combination thereof. The methods of the present invention are able to treat or prevent conditions of dendritic spine loss caused by a disease such as ALS.

Another embodiment of the present invention is a method of treating or preventing dendritic spine loss in a subject comprising the steps of administering a composition to a subject having or is at risk of having dendritic spine loss. The composition comprises an agent selected from the group comprising a salt, solvate, or stereoisomer of a norrin protein or functional part thereof; a vector expressing the norrin protein or functional part thereof; or a combination thereof. Then treating or preventing the dendritic spine loss in the subject.

Another embodiment of the present invention is a method of treating or preventing a condition of dendritic spine loss in a subject comprising the steps of administering an agonist of LGR6 to a subject having or at risk of having dendritic spine loss. Then treating or preventing the condition of dendritic spine loss in the subject.

Another embodiment of the present invention is a method of treating or preventing a condition of dendritic spine loss in a subject comprising the steps of administering a modulator of the expression of Norrin to a subject having or at risk of having dendritic spine loss Then treating or preventing the condition of dendritic spine loss.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “activity” refers to the ability of a gene to perform its function such as the Leucine-rich repeat containing G protein coupled receptor 6, or LGR6, to be a Wnt signaling pathway mediator.

The term “agent” refers to any small molecule chemical compound, antibody, nucleic acid molecule, or polypeptide, or fragments thereof.

The term “ALS” refers to amyotophic lateral sclerosis.

The term “alteration” refers to a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

The term “ameliorate” refers to decrease, suppress, attenuate, diminish, arrest, or stabilize the development or progression of a disease.

The term “analog” refers to a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

The term “disease” refers to any condition or disorder that damages or interferes with the normal function of a cell, tissue, or organ. Examples of diseases include a neurological disease such as dendritic spine loss in a subject and amyotrophic lateral sclerosis (ALS).

The term “effective amount” refers to the amount required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The term “express” refers to the ability of a gene to express the gene product including for example its corresponding mRNA or protein sequence (s).

The term “fragment” refers to a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

The term “GLT1” refers to a Glutamate Transporter. Glutamate transporters are a family of proteins that move glutamate—the principal excitatory neurotransmitter—across a membrane. The family of glutamate transporters is composed of two primary subclasses: the excitatory amino acid transporter (EAAT) family and vesicular glutamate transporter (VGLUT) family. In the brain, EAATs remove glutamate from the synaptic cleft and extra synaptic sites via glutamate reuptake into glial cells and neurons, while VGLUTs move glutamate from the cell cytoplasm into synaptic vesicles. Glutamate transporters also transport aspartate and are present in virtually all peripheral tissues, including the heart, liver, testes, and bone. They exhibit stereoselectivity for L-glutamate but transport both L-aspartate and D-aspartate.

The term “LGR6” refers to a Leucine Rich Repeat Containing G Protein Coupled Receptor 6 or the gene that expresses this protein.

The term “Norrin” refers to also known as Norrie disease protein or X-linked exudative vitreoretinopathy 2 protein (EVR2) is a protein that in humans is encoded by the NDP gene. Mutations in the NDP gene are associated with the Norrie disease. Examples of Norrin proteins and nucleic sequences are:

| NDP | Human | Mouse |
|---|---|---|
| RefSeq (mRNA) | NM 000266 | NM010883 |
| RefSeq (protein) | NP 000257 | NP 035013 |

For examples:

| Homo sapiens NDP, norrin cystine knot growth factor (NDP) | |
|---|---|
| Homo sapien NDP protein | |
| Sequence ID: NM 000266.3 | |
| SEQ ID NO: 1 | |
| MRKHVLAASFSMLSLLVIMGDTDSKTDSSFIMDSDPRRCMRHHYVDSISHPLYKCSSKMVLLARCEGHCS | |
| QASRSEPLVSFSTVLKQPFRSSCHCCRPQTSKLKALRLRCSGGMRLTATYRYILSCHCEECNS | |
| Homo sapien NDP DNA Sequences [The bold nucleic acids are | |
| the coding or cDNA Sequences] | |
| ORIGIN | |
| SEQ ID NO: 2 | |
| 1 | aagatgctcc gtggaaggga gccgagcggt gggcagaggc tgagtccccg ataacgagcg |
| 61 | cctcacattt ccgtggcatt cccatttgct agtgcgctgc tgcggccgca cgcctgattg |
| 121 | atatatgact gcaatggcac ttttccattt gacattctct ctctctctct ccctctctct |
| 181 | ctctccctct ctctctccct ctctctctct ccctgtgtcg cttaaacaac agtcctaact |
| 241 | tttgtgtgtt gcaaatataa aaggcaagcc atgtgacaga gggacagaag aacaaaagca |
| 301 | tttggaagta acaggacctc tttctagctc tcagaaaagt ctgagaagaa aggagccctg |
| 361 | cgttccccta agctgtgcag cagatactgt gatgatggat tgcaagtgca aagagtaaga |
| 421 | caaaactcca gcacataaag gacaatgaca accagaaagc ttcagcccga tcctgccctt |
| 481 | tccttgaacg ggactggatc ctaggaggtg aagccatttc caattttttg tcctctgcct |
| 541 | ccctctgctg ttcttctaga gaagtttttc cttacaaca [symbol] [symbol] [symbol] |
| 601 | [symbol] [symbol] [symbol] [symbol] [symbol] [symbol] |
| 661 | [symbol] [symbol] [symbol] [symbol] [symbol] [symbol] |
| 721 | [symbol] [symbol] [symbol] [symbol] [symbol] [symbol] |
| 781 | [symbol] [symbol] [symbol] [symbol] [symbol] [symbol] |
| 841 | [symbol] [symbol] [symbol] [symbol] [symbol] [symbol] |
| 901 | [symbol] [symbol] [symbol] [symbol] [symbol] [symbol] |
| 961 | [symbol] [symbol] ggcccgctg ctgtgtgtgg cttctggatg ggacaactgt |
| 1021 | agaggcagtt cgaccagcca gggaaagact ggcaagaaaa gagttaaggc aaaaaaggat |
| 1081 | gcaacaattc tcccgggact ctgcatattc tagtaataaa gactctacat gcttgttgac |
| 1141 | agagagagat actctgggaa cttctttgca gttcccatct cctttctctg gtacaatttc |
| 1201 | ttttggttca ttttcagatt caggcatttt cccccttggc tctcaatgct gtttgggttt |
| 1261 | ccaacaattc agcattagtg ggaaaaagtg ggccctcata cacaagcgtg tcaggctgtc |
| 1321 | agtgtttggt gcacgctggg gaagaattta ctttggaaag tagaaaagcc cagcttttcc |
| 1381 | tgggacatct tctgttattg ttgatgtttt tttttacctt gtcattttgg tctaaggttg |
| 1441 | ccattgctgc taaaggttac cgatttcaaa gtccagatac caagcatgtg gatatgttta |
| 1501 | gctacgttta ctcacagcca gcgaactgac attaaaataa ctaacaaaca gattctttta |
| 1561 | tgtgatgctg gaactcttga cagctataat tattattcag aaatgacttt ttgaaagtaa |
| 1621 | aagcagcata aagaatttgt cacaggaagg ctgtctcaga taaattatgg taaaattttg |
| 1681 | taagggagca gacttttaaa gacttgcaca aatacggatc ctgcactgac tctggaaaag |
| 1741 | gcatatatgt actagtggca tggagaatgc accatactca tgcatgcaaa ttagacaacc |
| 1801 | aagtatgaat ctatttgtgg gtgtgctata gctttagccg tgtcacgggc atcattctct |
| 1861 | aatatccact tgtccatgtg aaacatgttg ccaaaatggt ggcctggctt gtcttctgaa |
| 1921 | cgtttggttc aaatgtgttt tggtcctgga ggctcaaatt ttgagttatt cccacgtttt |
| 1981 | gaaataaaaa gagtatattc aaaaaaaaaa aaaaaaaaaa aaaaaaaaaa aaaaaaaaaa |
| 2041 | aaaaaaaaaa aaaaaaaa |
| Mus musculus Norrie disease (pseudoglioma) (human?) (Ndp), | |
| cDNA | |
| NCB 1 Reference Sequence: NM_010683.3 | |
| >NM_010883.3 Mus musculus Norrie disease (pseudoglioma) | |
| (human) (Ndp), cDNA | |
| SEQ ID NO: 3 | |
| CTCATCTTAAGATGCTCCATGGAAAAGATCCGAGCAGTGGGCAGAGGCTGTGAGTCCCCGATAACGAGAG | |
| CGCCTAACATTTCCGTGGTATTCCCATTTGCTAGAGCGCTGCTGCGGCCACACGCCTGATTGATATATGC | |
| CTGCAATGGCACTTTTCCATTTGACACTCTCCCTCTCTCTCTCCCTCTCTCTCCCTCTCTCTCTCTCCCT | |
| CTCTCTCCCTCTCTCTCCCTGGGTCGCTTAAACAACAGTCCTAACTTTTGTGTGTTGCAAATATAAAAGG | |
| CAAGCCATGTGACAGAGGGACAGAAGAACAAAAGCATTTGGAAGTAACAGGACCTCTTTCTAGCTCTCAG | |
| AAAAGTCTGAGAAGAAAGGAGCCCTGCGTTTCCCCCAAGCTGTGCAGCACATACTGCTGTGATCGGTTGC | |
| AAGTGAAAGAAGCAAGAGAAATTCCTGCATACAAAGGACAATGACAACCAGAAAGCGGCAGCCCTATCCT | |
| GCTCTGGCCTTGAAAGGGACTGGATCCTAGGAGGTGGCAGCATTTCCAATCTATTGTCCTCTGTCTCCCT | |
| CTGCTGTTTTTCTGGAGGAGTTTCCCTTTACAACAATGAGAAATCATGTACTAGCTGCATCCATTTCTAT | |
| GCTCTCCCTGCTGGCCATAATGGGAGATACAGACAGCAAAACAGACAGTTCATTTCTGATGGACTCTCAA | |
| CGCTGCATGAGACACCATTATGTCGATTCTATCAGTCACCCACTGTACAAATGTAGCTCAAAGATGGTGC | |
| TCCTGGCCAGATGTGAGGGGCACTGCAGCCAGGCATCACGCTCTGAGCCCTTGGTGTCCTTCAGCACTGT | |
| CCTCAAGCAACCTTTCCGTTCCTCCTGTCACTGCTGCCGACCCCAGACTTCCAAGCTGAAGGCTCTGCGT | |
| CTGCGCTGCTCAGGGGGCATGCGACTTACTGCCACTTACCGGTACATCCTCTCCTGTCACTGTGAGGAAT | |
| GCAGCTCCTGAGACTTGCTGATGATTGGCTTTCTGACTGGCACAACCACAGGAGCAGTTCAACCTGCCAG | |
| AGACGGACTGGCAAGAAAAGAGTTAAGGCAGATAAAGATGGAGCAAGTCCCATAGGATTTTGCATATTCT | |
| TGTCCTAAAGACTCAATGTGCTTTTGACAGAAAGTGACTCTGGGAACTTGCTTTTCATTCCCATCTCCTT | |
| TCCCTGGAAGAATTTCTTTTGGTTACTTTACAGATTCAGGCATTTCCCCTGTTGGCTCTAATTGTGGTTT | |
| GGGTGCCTGACAGCTCTGCATTAGTGGGAAAATGTGGGGCCCTGTGCAATAGCATGTCAGGCTGTTCCTA | |
| TTTGGTGCATATTAGGGAAAATTTTACCTAACTCTCCTTAGGAGATCTTTGCTTGTTGTTTCCCCTGGTC | |
| ATTTTGGTCTAAGATTTGCCTCTAAAGTTTCCTGGTTTCAAGATCTGGACACCCAGTCCATGGATGTTTA | |
| GTGAGGCTTACTCACAGCCAGCTAACTGCTACTAAAATAACTAACACATGGGTTCTTTTATGTGACAGCG | |
| GGACTCCTGACCACTATAGTAATTATTCAGAAGTGACTGAGGGGATATAAATGTGGCAGAGGAATTTATA | |
| ATCTGAAGCCTTTTGTGAGGAAGCAGGCTTTCACACATACACACTCAGGTGGATCCTGCACTGACTCTGG | |
| AGAAGGCATACATTATACTTGGTGTGGAGAACACACCATACTCATAATTGAGCATTAGTCAAGCATGTAG | |
| GAATCTACTTGTGGGTGTGCAATAGCTTCAGCCATATCTTAGCTATATCCACCTGTCTATGTGAAGCTTG | |
| TTGCCGTAGTGGTGGCCCGACTTATTGTCTGAAATTTTTGTTTCAATATATTTTTGGTCCTCGAAGCTCA | |
| AATTTTGAAGTCTCCCCATGTTTTCAAATAAAAATAGAGTACCTTCAAAAAAAAAAAAAAAAAAAAAAAA | |
| AA | |
| Mouse NDP cDNA Protein: | |
| SEQ ID NO: 4 | |
| MrnhvlaasismlsllaimgdtdsktdssflmdsqrcmrhhyVdsishplykcsskmyllarceghcsqa | |
| srseplvsfstvlkqpfrsschccrpqtsklkalrlrcsggmrltatyryilschceecss* | |
| Mouse NDP cDNA (coded protein disclosed as SEQ ID NO: 4): | |
| SEQ ID NO:5 | |
| | PstI |
| | ~~~~~~ |
| 1 | AGCAAGCACA GGTGGCAGCG GCTGCAGGGG CGCATCGCCG GCGTGCGCCC |
| | PstI AvaI |
| | ~~~~~~ ~~~~~~~ |
| 51 | TCCTGCAGCC CTGGGCGCAT CGCTCTCTCG GGGAAGCCAC CCTCGGAGCC |
| | HindIII |
| | ~~~~~~~ |
| | M R N H • |
| 101 | CCCGGAGCTC CCCGCCAAGC GCCATAAGCT TCGATCGCCA TGAGAAATCA |
| | • V L A A S I S M L S L L A I M G D • |
| 151 | TGTACTAGCT GCATCCATTT CTATGCTCTC CCTGCTGGCC ATAATGGGAG |
| | • T D S K T D S S F L M D S Q R C |
| 201 | ATACAGACAG CAAAACAGAC AGTTCATTTC TGATGGACTC TCAACGCTGC |
| | M R H H Y V D S I S H P L Y K C S • |
| 251 | ATGAGACACC ATTATGTCGA TTCTATCAGT CACCCACTGT ACAAATGTAG |
| | PstI |
| | ~~~~~~~ |
| | • S K M V L L A R C E G H C S Q A S • |
| 301 | CTCAAAGATG GTGCTCCTGG CCAGATGTGA GGGGCACTGC AGCCAGGCAT |
| | • R S E P L V S F S T V L K Q P F |
| 351 | CACGCTCTGA GCCCTTGGTG TCCTTCAGCA CTGTCCTCAA GCAACCTTTC |
| | R S S C H C C R P Q T S K L K A L • |
| 401 | CGTTCCTCCT GTCACTGCTG CCGACCCCAG ACTTCCAAGC TGAAGGCTCT |
| | • R L R C S G G M R L T A T Y R Y I • |
| 451 | GCGTCTGCGC TGCTCAGGGG GCATGCGACT TACTGCCACT TACCGGTACA |
| | • L S C H C E E C S S * |
| 501 | TCCTCTCCTG TCACTGTGAG GAATGCAGCT CCTGAACGCG TACGCGGCCG |
| | AvaI |
| | ~~~~~~ |
| 551 | CTCGAGAGCG CTAAGCTCCG AGGGCCGCCA CCACCTGTTC CTGTACGGCA |
| | EcoRI BamHI |
| | ~~~~~~ ~~~~~~ |
| 601 | TGGACGAGCT GTACAAGTAA GAATTCGATA TCGGATCCGC AGATCCCGGC |
| 651 | CAGATACCGA TGCTGCCGCA GCAAAAGCAG GAGCAGATGC CGCCGTCGCA |
| 701 | GGCGAAGATG TCGCAGACGG AGGAGGCGAT GCTGCCGGCG GAGGAGGCGA |
| 751 | AGTAAGTAGA GGGCTGGGCT GGGCTGTGGG GGGTGTGGGG TGCGGGACTG |
| 801 | GGCAGTCTGG GAGTCCCTCT CACCACTTTT CTTACCTTTC TAGGATGCTG |
| 851 | CCGTCGCCGC CGCTCATACA CCATAAGGTG TAAAAANTAC TAGATGCACA |
| 901 | GAATAGCAAG TCCATCAAAA CTCCTGCGTG AGAATTTTAC CAGACTTCAA |
| 951 | GAGCATCTCG CCACATCTTG AAAAATGCCA CCGTCCGATG AAAAACAGGA |
| 1001 | GCCTGCTAAG GAACAATGCC ACCTGTCAAT AAATGTTGAA AACTCATCCC |
| 1051 | ATTCCTGCCT CTTGGTCCTT GGGCTT |

norrin precursor [Homo sapiens]
NCBI Reference Sequence: NP_000257.1
>NP_000257.1 norrin precursor [Homo sapiens]
SEQ ID NO: 6
MRKHVLAASFSMLSLLVIMGDTDSKTDSSFIMDSDPRRCMRHHYVDSISHPLYKCSSKMVLLARCEGHCS
QASRSEPLVSFSTVLKQPFRSSCHCCRPQTSKLKALRLRCSGGMRLTATYRYILSCHCEECNS
norrin precursor [Mus musculus]
NCBI Reference Sequence: NP_035013:1
>NP_035013.1 norrin precursor [Mus musculus]
SEQ ID NO: 7
MRNHVLAASISMLSLLAIMGDTDSKTDSSFLMDSQRCMRHHYVDSISHPLYKCSSKMVLLARCEGHCSQA
SRSEPLVSFSTVLKQPFRSSCHCCRPQTSKLKALRLRCSGGMRLTATYRYILSCHCEECSS

The term, “obtaining” as in “obtaining an agent” includes synthesizing, purchasing, or otherwise acquiring the agent.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

The term “reduces” refers to a negative alteration of at least 10%, 25%, 50%, 75%, or 100%.

A “reference” refers to a standard or control conditions such as a sample (human cells) or a subject that is a free, or substantially free, of an agent such as one or more agonist of LGR6, including RSPO1, for example.

A “reference sequence” is a defined sequence used as a basis for sequence comparison. A reference sequence may be a subset of or the entirety of a specified sequence;

for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or there between.

The term “RSPO1” refers to R-spondin 1 or a secreted protein that in humans is encoded by the Rspo1 gene, found on chromosome 1. In humans, it interacts with WNT4 in the process of female sex development. Loss of function can cause female to male sex reversal. Furthermore, it promotes canonical WNT/β catenin signaling. An example of a human RSPO1 gene may be found on the NCBI database having Gene ID number 284654 and an example of a human RSPO 1 protein sequence may be found on the NCBI database having Accession number: XP-006710646.1.

Homo sapiens R-spondin 1 (RSPO1), transcript variant 1, cDNA
NCBI Reference Sequence; NM_001038633.3
>NM_001038633.3 Homo sapiens R-spondin 1 (RSPO1), transcript
variant 1, cDNA
SEQ ID NO: 8
ATTCCCTCCCTGGTGCTCGCAGAGGACTGGCCCCTCTCCGGGCTGGGAGCTCCGGCCGAGCGGAGGCGCG
ACGGAGAGCACCAGCGCAGGGCAGAGAGCCCGGAGCGACCGGCCAGAGTAGGGCATCCGCTCGGGTGCTG
CGGAGAACGAGGGCAGCTCCGAGCCGCCCCGGAGGACCGATGCGCCGGGTGGGGCGCTGGCCCCGAGGGC
GTGAGCCGTCCGCAGATTGAGCAACTTGGGAACGGGCGGGCGGAGCGCAGGCGAGCCGGGCGCCCAGGAC
AGTCCCGCAGCGGGCGGGTGAGCGGGCCGCGCCCTCGCCCCTCCCGGGCCTGCCCCCGTCGCGACTGGCA
GCACGAAGCTGAGATTGTGGTTTCCTGGTGATTCAGGTGGGAGTGGGCCAGAAGATCACCGCTGGCAAGG
ACTGGTGTTTGTCAACTGTAAGGACTCATGGAACAGATCTACCAGGGATTCTCAGACCTTAGTTTGAGAA
ATGCTGCAATTAAAGGCAAATCCTATCACTCTGAGTGATCGCTTTGGTGTCGAGGCAATCAACCATAAAG
ATAAATGCAAATATGGAAATTGCATAACAGTACTCAGTATTAAGGTTGGTTTTTGGAGTAGTCCCTGCTG
ACGTGACAAAAAGATCTCTCATATGATATTCCGAGGTATCTTTGAGGAAGTCTCTCTTTGAGGACCTCCC
TTTGAGCTGATGGAGAACTGGGCTCCCCACACCCTCTCTGTCCCCAGCTGAGATTATGGTGGATTTGGGC
TACGGCCCAGGCCTGGGCCTCCTGCTGCTGACCCAGCCCCAGAGGTGTTAGCAAGAGCCGTGTGCTATCC
ACCCTCCCCGAGACCACCCCTCCGACCAGGGGCCTGGAGCTGGCGCGTGACTATGCGGCTTGGGCTGTGT
GTGGTGGCCCTGGTTCTGAGCTGGACGCACCTCACCATCAGCAGCCGGGGGATCAAGGGGAAAAGGCAGA
GGCGGATCAGTGCCGAGGGGAGCCAGGCCTGTGCCAAAGGCTGTGAGCTCTGCTCTGAAGTCAACGGCTG
CCTCAAGTGCTCACCCAAGCTGTTCATCCTGCTGGAGAGGAACGACATCCGCCAGGTGGGCGTCTGCTTG
CCGTCCTGCCCACCTGGATACTTCGACGCCCGCAACCCCGACATGAACAAGTGCATCAAATGCAAGATCG
AGCACTGTGAGGCCTGCTTCAGCCATAACTTCTGCACCAAGTGTAAGGAGGGCTTGTACCTGCACAAGGG
CCGCTGCTATCCAGCTTGTCCCGAGGGCTCCTCAGCTGCCAATGGCACCATGGAGTGCAGTAGTCCTGCG
CAATGTGAAATGAGCGAGTGGTCTCCGTGGGGGCCCTGCTCCAAGAAGCAGCAGCTCTGTGGTTTCCGGA
GGGGCTCCGAGGAGCGGACACGCAGGGTGCTACATGCCCCTGTGGGGGACCATGCTGCCTGCTCTGACAC
CAAGGAGACCCGGAGGTGCACAGTGAGGAGAGTGCCGTGTCCTGAGGGGCAGAAGAGGAGGAAGGGAGGC
CAGGGCCGGCGGGAGAATGCCAACAGGAACCTGGCCAGGAAGGAGAGCAAGGAGGCGGGTGCTGGCTCTC
GAAGACGCAAGGGGCAGCAACAGCAGCAGCAGCAAGGGACAGTGGGGCCACTCACATCTGCAGGGCCTGC
CTAGGGACACTGTCCAGCCTCCAGGCCCATGCAGAAAGAGTTCAGTGCTACTCTGCGTGATTCAAGCTTT
CCTGAACTGGAACGTCGGGGGCAAAGCATACACACACACTCCAATCCATCCATGCATACATAGACACAAG
ACACACACGCTCAAACCCCTGTCCACATATACAACCATACATACTTGCACATGTGTGTTCATGTACACAC
GCAGACACAGACACCACACACACACATACACACACACACACACACACACCTGAGGCCACCAGAAGACACT
TCCATCCCTCGGGCCCAGCAGTACACACTTGGTTTCCAGAGCTCCCAGTGGACATGTCAGAGACAACACT
TCCCAGCATCTGAGACCAAACTGCAGAGGGGAGCCTTCTGGAGAAGCTGCTGGGATCGGACCAGCCACTG
TGGCAGATGGGAGCCAAGCTTGAGGACTGCTGGTGACCTGGGAAGAAACCTTCTTCCCATCCTGTTCAGC
ACTCCCAGCTGTGTGACTTTATCGTTGGAGAGTATTGTTACCCTTCCAGGATACATATCAGGGTTAACCT
GACTTTGAAAACTGCTTAAAGGTTTATTTCAAATTAAAACAAAAAAATCAACGACAGCAGTAGACACAGG
CACCACATTCCTTTGCAGGGTGTGAGGGTTTGGCGAGGTATGCGTAGGAGCAAGAAGGGACAGGGAATTT
CAAGAGACCCCAAATAGCCTGCTCAGTAGAGGGTCATGCAGACAAGGAAGAAAACTTAGGGGCTGCTCTG
ACGGTGGTAAACAGGCTGTCTATATCCTTGTTACTCAGAGCATGGCCCGGCAGCAGTGTTGTCACAGGGC
AGCTTGTTAGGAATGAGAATCTCAGGTCTCATTCCAGACCTGGTGAGCCAGAGTCTAAATTTTAAGATTC
CTGATGATTGGCATGTTACCCAAATTTGAGAAGTGCTGCTGTAATTCCCCTTAAAGGACGGGAGAAAGGG
CCCCGGCCATCTTGCAGCAGGAGGGATTCTGGTCAGCTATAPAGGAGGACTTTCCATCTGGGAGAGGCAG
AATCTATATACTGAAGGGCTAGTGGCACTGCCAGGGGAAGGGAGTGCGTAGGCTTCCAGTGATGGTTGGG
GACAATCCTGCCCAAAGGCAGGGCAGTGGATGGAATAACTCCTTGTGGCATTCTGAAGTGTGTGCCAGGC
TCTGGACTAGGTGCTAGGTTTCCAGGGAGGAGCCAAACACGGGCCTTGCTCTTGTGGAGCTTAGAGGTTG
GTGGGGAAGAAAATAGGCATGCACCAAGGAATTGTACAAACACATATATAACTACAAAAGGATGGTGCCA
AGGGCAGGTGACCACTGGCATCTATGCTTAGCTATGAAAGTGAATAAAGCAGAATAAAAATAAAATACTT
TCTCTCAGG
Homo sapiens R-spondin 1 (RSPO1), transcript variant 2, cDNA
NCBI Reference Sequence: NM_001242908.1
>NM_001242908.1 Homo sapiens R-spondin 1 (RSPO1), transcript
variant 2, cDNA
SEQ ID NO:9
ATTCCCTCCCTGGTGCTCGCAGAGGACTGGCCCCTCTCCGGGCTGGGAGCTCCGGCCGAGCGGAGGCGCG
ACGGAGAGCACCAGCGCAGGGCAGAGAGCCCGGAGCGACCGGCCAGAGTAGGGCATCCGCTCGGGTGCTG
CGGAGAACGAGGGCAGCTCCGAGCCGCCCCGGAGGACCGATGCGCCGGGTGGGGCGCTGGCCCCGAGGGC
GTGAGCCGTCCGCAGATTGAGCAACTTGGGAACGGGCGGGCGGAGCGCAGGCGAGCCGGGCGCCCAGGAC
AGTCCCGCAGCGGGCGGGTGAGCGGGCCGCGCCCTCGCCCCTCCCGGGCCTGCCCCCGTCGCGACTGGCA
GCACGAAGCTGAGATTGTGGTTTCCTGGTGATTCAGGTGGGAGTGGGCCAGAAGATCACCGCTGGCAAGG
ACTGGGTTGGTTTTTGGAGTAGTCCCTGCTGACGTGACAAAAAGATCTCTCATATGATATTCCGAGGTAT
CTTTGAGGAAGTCTCTCTTTGAGGACCTCCCTTTGAGCTGATGGAGAACTGGGCTCCCCACACCCTCTCT
GTCCCCAGCTGAGATTATGGTGGATTTGGGCTACGGCCCAGGCCTGGGCCTCCTGCTGCTGACCCAGCCC
CAGAGGTGTTAGCAAGAGCCGTGTGCTATCCACCCTCCCCGAGACCACCCCTCCGACCAGGGGCCTGGAG
CTGGCGCGTGACTATGCGGCTTGGGCTGTGTGTGGTGGCCCTGGTTCTGAGCTGGACGCACCTCACCATC
AGCAGCCGGGGGATCAAGGGGAAAAGGCAGAGGCGGATCAGTGCCGAGGGGAGCCAGGCCTGTGCCAAAG
GCTGTGAGCTCTGCTCTGAAGTCAACGGCTGCCTCAAGTGCTCACCCAAGCTGTTCATCCTGCTGGAGAG
GAACGACATCCGCCAGGTGGGCGTCTGCTTGCCGTCCTGCCCACCTGGATACTTCGACGCCCGCAACCCC
GACATGAACAAGTGCATCAAATGCAAGATCGAGCACTGTGAGGCCTGCTTCAGCCATAACTTCTGCACCA
AGTGTAAGGAGGGCTTGTACCTGCACAAGGGCCGCTGCTATCCAGCTTGTCCCGAGGGCTCCTCAGCTGC
CAATGGCACCATGGAGTGCAGTAGTCCTGCGCAATGTGAAATGAGCGAGTGGTCTCCGTGGGGGCCCTGC
TCCAAGAAGCAGCAGCTCTGTGGTTTCCGGAGGGGCTCCGAGGAGCGGACACGCAGGGTGCTACATGCCC
CTGTGGGGGACCATGCTGCCTGCTCTGACACCAAGGAGACCCGGAGGTGCACAGTGAGGAGAGTGCCGTG
TCCTGAGGGGCAGAAGAGGAGGAAGGGAGGCCAGGGCCGGCGGGAGAATGCCAACAGGAACCTGGCCAGG
AAGGAGAGCAAGGAGGCGGGTGCTGGCTCTCGAAGACGCAAGGGGCAGCAACAGCAGCAGCAGCAAGGGA
CAGTGGGGCCACTCACATCTGCAGGGCCTGCCTAGGGACACTGTCCAGCCTCCAGGCCCATGCAGAAAGA
GTTCAGTGCTACTCTGCGTGATTCAAGCTTTCCTGAACTGGAACGTCGGGGGCAAAGCATACACACACAC
TCCAATCCATCCATGCATACATAGACACAAGACACACACGCTCAAACCCCTGTCCACATATACAACCATA
CATACTTGCACATGTGTGTTCATGTACACACGCAGACACAGACACCACACACACACATACACACACACAC
ACACACACACCTGAGGCCACCAGAAGACACTTCCATCCCTCGGGCCCAGCAGTACACACTTGGTTTCCAG
AGCTCCCAGTGGACATGTCAGAGACAACACTTCCCAGCATCTGAGACCAAACTGCAGAGGGGAGCCTTCT
GGAGAAGCTGCTGGGATCGGACCAGCCACTGTGGCAGATGGGAGCCAAGCTTGAGGACTGCTGGTGACCT
GGGAAGAAACCTTCTTCCCATCCTGTTCAGCACTCCCAGCTGTGTGACTTTATCGTTGGAGAGTATTGTT
ACCCTTCCAGGATACATATCAGGGTTAACCTGACTTTGAAAACTGCTTAAAGGTTTATTTCAAATTAAAA
CAAAAAAATCAACGACAGCAGTAGACACAGGCACCACATTCCTTTGCAGGGTGTGAGGGTTTGGCGAGGT
ATGCGTAGGAGCAAGAAGGGACAGGGAATTTCAAGAGACCCCAAATAGCCTGCTCAGTAGAGGGTCATGC
AGACAAGGAAGAAAACTTAGGGGCTGCTCTGACGGTGGTAAACAGGCTGTCTATATCCTTGTTACTCAGA
GCATGGCCCGGCAGCAGTGTTGTCACAGGGCAGCTTGTTAGGAATGAGAATCTCAGGTCTCATTCCAGAC
CTGGTGAGCCAGAGTCTAAATTTTAAGATTCCTGATGATTGGCATGTTACCCAAATTTGAGAAGTGCTGC
TGTAATTCCCCTTAAAGGACGGGAGAAAGGGCCCCGGCCATCTTGCAGCAGGAGGGATTCTGGTCAGCTA
TAAAGGAGGACTTTCCATCTGGGAGAGGCAGAATCTATATACTGAAGGGCTAGTGGCACTGCCAGGGGAA
GGGAGTGCGTAGGCTTCCAGTGATGGTTGGGGACAATCCTGCCCAAAGGCAGGGCAGTGGATGGAATAAC
TCCTTGTGGCATTCTGAAGTGTGTGCCAGGCTCTGGACTAGGTGCTAGGTTTCCAGGGAGGAGCCAAACA
CGGGCCTTGCTCTTGTGGAGCTTAGAGGTTGGTGGGGAAGAAAATAGGCATGCACCAAGGAATTGTACAA
ACACATATATAACTACAAAAGGATGGTGCCAAGGGCAGGTGACCACTGGCATCTATGCTTAGCTATGAAA
GTGAATAAAGCAGAATAAAAATAAAATACTTTCTCTCAGG
Homo sapiens R-spondin 1 (RSPO1), transcript variant 3, cDNA
NCBI Reference Sequence. NM_001242909.1
>NM_001242909.1 Homo sapiens R-spondin 1 (RSPO1), transcript
variant 3, cDNA
SEQ ID NO: 10
ATTCCCTCCCTGGTGCTCGCAGAGGACTGGCCCCTCTCCGGGCTGGGAGCTCCGGCCGAGCGGAGGCGCG
ACGGAGAGCACCAGCGCAGGGCAGAGAGCCCGGAGCGACCGGCCAGAGTAGGGCATCCGCTCGGGTGCTG
CGGAGAACGAGGGCAGCTCCGAGCCGCCCCGGAGGACCGATGCGCCGGGTGGGGCGCTGGCCCCGAGGGC
GTGAGCCGTCCGCAGATTGAGCAACTTGGGAACGGGCGGGCGGAGCGCAGGCGAGCCGGGCGCCCAGGAC
AGTCCCGCAGCGGGCGGGTGAGCGGGCCGCGCCCTCGCCCCTCCCGGGCCTGCCCCCGTCGCGACTGGCA
GCACGAAGCTGAGATTGTGGTTTCCTGGTGATTCAGGTGGGAGTGGGCCAGAAGATCACCGCTGGCAAGG
ACTGGGTTGGTTTTTGGAGTAGTCCCTGCTGACGTGACAAAAAGATCTCTCATATGATATTCCGAGTCAG
TGCCGAGGGGAGCCAGGCCTGTGCCAAAGGCTGTGAGCTCTGCTCTGAAGTCAACGGCTGCCTCAAGTGC
TCACCCAAGCTGTTCATCCTGCTGGAGAGGAACGACATCCGCCAGGTGGGCGTCTGCTTGCCGTCCTGCC
CACCTGGATACTTCGACGCCCGCAACCCCGACATGAACAAGTGCATCAAATGCAAGATCGAGCACTGTGA
GGCCTGCTTCAGCCATAACTTCTGCACCAAGTGTAAGGAGGGCTTGTACCTGCACAAGGGCCGCTGCTAT
CCAGCTTGTCCCGAGGGCTCCTCAGCTGCCAATGGCACCATGGAGTGCAGTAGTCCTGCGCAATGTGAAA
TGAGCGAGTGGTCTCCGTGGGGGCCCTGCTCCAAGAAGCAGCAGCTCTGTGGTTTCCGGAGGGGCTCCGA
GGAGCGGACACGCAGGGTGCTACATGCCCCTGTGGGGGACCATGCTGCCTGCTCTGACACCAAGGAGACC
CGGAGGTGCACAGTGAGGAGAGTGCCGTGTCCTGAGGGGCAGAAGAGGAGGAAGGGAGGCCAGGGCCGGC
GGGAGAATGCCAACAGGAACCTGGCCAGGAAGGAGAGCAAGGAGGCGGGTGCTGGCTCTCGAAGACGCAA
GGGGCAGCAACAGCAGCAGCAGCAAGGGACAGTGGGGCCACTCACATCTGCAGGGCCTGCCTAGGGACAC
TGTCCAGCCTCCAGGCCCATGCAGAAAGAGTTCAGTGCTACTCTGCGTGATTCAAGCTTTCCTGAACTGG
AACGTCGGGGGCAAAGCATACACACACACTCCAATCCATCCATGCATACATAGACACAAGACACACACGC
TCAAACCCCTGTCCACATATACAACCATACATACTTGCACATGTGTGTTCATGTACACACGCAGACACAG
ACACCACACACACACATACACACACACACACACACACACCTGAGGCCACCAGAAGACACTTCCATCCCTC
GGGCCCAGCAGTACACACTTGGTTTCCAGAGCTCCCAGTGGACATGTCAGAGACAACACTTCCCAGCATC
TGAGACCAAACTGCAGAGGGGAGCCTTCTGGAGAAGCTGCTGGGATCGGACCAGCCACTGTGGCAGATGG
GAGCCAAGCTTGAGGACTGCTGGTGACCTGGGAAGAAACCTTCTTCCCATCCTGTTCAGCACTCCCAGCT
GTGTGACTTTATCGTTGGAGAGTATTGTTACCCTTCCAGGATACATATCAGGGTTAACCTGACTTTGAAA
ACTGCTTAAAGGTTTATTTCAAATTAAAACAAAAAAATCAACGACAGCAGTAGACACAGGCACCACATTC
CTTTGCAGGGTGTGAGGGTTTGGCGAGGTATGCGTAGGAGCAAGAAGGGACAGGGAATTTCAAGAGACCC
CAAATAGCCTGCTCAGTAGAGGGTCATGCAGACAAGGAAGAAAACTTAGGGGCTGCTCTGACGGTGGTAA
ACAGGCTGTCTATATCCTTGTTACTCAGAGCATGGCCCGGCAGCAGTGTTGTCACAGGGCAGCTTGTTAG
GAATGAGAATCTCAGGTCTCATTCCAGACCTGGTGAGCCAGAGTCTAAATTTTAAGATTCCTGATGATTG
GCATGTTACCCAAATTTGAGAAGTGCTGCTGTAATTCCCCTTAAAGGACGGGAGAAAGGGCCCCGGCCAT
CTTGCAGCAGGAGGGATTCTGGTCAGCTATAAAGGAGGACTTTCCATCTGGGAGAGGCAGAATCTATATA
CTGAAGGGCTAGTGGCACTGCCAGGGGAAGGGAGTGCGTAGGCTTCCAGTGATGGTTGGGGACAATCCTG
CCCAAAGGCAGGGCAGTGGATGGAATAACTCCTTGTGGCATTCTGAAGTGTGTGCCAGGCTCTGGACTAG
GTGCTAGGTTTCCAGGGAGGAGCCAAACACGGGCCTTGCTCTTGTGGAGCTTAGAGGTTGGTGGGGAAGA
AAATAGGCATGCACCAAGGAATTGTACAAACACATATATAACTACAAAAGGATGGTGCCAAGGGCAGGTG
ACCACTGGCATCTATGCTTAGCTATGAAAGTGAATAAAGCAGAATAAAAATAAAATACTTTCTCTCAGG
Homo sapiens R-spondin 1 (RSPO1), transcript variant 4, cDNA
NCBI Reference Sequence. NM_001242910.1
>NM_001242910.1 Homo sapiens R-spondin 1 (RSPO1), transcript
variant 4, cDNA
SEQ ID NO: 11
ATTCCCTCCCTGGTGCTCGCAGAGGACTGGCCCCTCTCCGGGCTGGGAGCTCCGGCCGAGCGGAGGCGCG
ACGGAGAGCACCAGCGCAGGGCAGAGAGCCCGGAGCGACCGGCCAGAGTAGGGCATCCGCTCGGGTGCTG
CGGAGAACGAGGGCAGCTCCGAGCCGCCCCGGAGGACCGATGCGCCGGGTGGGGCGCTGGCCCCGAGGGC
GTGAGCCGTCCGCAGATTGAGCAACTTGGGAACGGGCGGGCGGAGCGCAGGCGAGCCGGGCGCCCAGGAC
AGTCCCGCAGCGGGCGGGTGAGCGGGCCGCGCCCTCGCCCCTCCCGGGCCTGCCCCCGTCGCGACTGGCA
GCACGAAGCTGAGATTGTGGTTTCCTGGTGATTCAGGTGGGAGTGGGCCAGAAGATCACCGCTGGCAAGG
ACTGGGTTGGTTTTTGGAGTAGTCCCTGCTGACGTGACAAAAAGATCTCTCATATGATATTCCGAGGTAT
CTTTGAGGAAGTCTCTCTTTGAGGACCTCCCTTTGAGCTGATGGAGAACTGGGCTCCCCACACCCTCTCT
GTCCCCAGCTGAGATTATGGTGGATTTGGGCTACGGCCCAGGCCTGGGCCTCCTGCTGCTGACCCAGCCC
CAGAGGTGTTAGCAAGAGCCGTGTGCTATCCACCCTCCCCGAGACCACCCCTCCGACCAGGGGCCTGGAG
CTGGCGCGTGACTATGCGGCTTGGGCTGTGTGTGGTGGCCCTGGTTCTGAGCTGGACGCACCTCACCATC
AGCAGCCGGGGGATCAAGGGGAAAAGGCAGAGGCGGATCAGTGCCGAGGGGAGCCAGGCCTGTGCCAAAG
GCTGTGAGCTCTGCTCTGAAGTCAACGGCTGCCTCAAGTGCTCACCCAAGCTGTTCATCCTGCTGGAGAG
GAACGACATCCGCCAGGTGGGCGTCTGCTTGCCGTCCTGCCCACCTGGATACTTCGACGCCCGCAACCCC
GACATGAACAAGTGCATCAAATGCAAGATCGAGCACTGTGAGGCCTGCTTCAGCCATAACTTCTGCACCA
AGTGTAAGGAGGGCTTGTACCTGCACAAGGGCCGCTGCTATCCAGCTTGTCCCGAGGGCTCCTCAGCTGC
CAATGGCACCATGGAGTGCAGTAGTCCTGGGCAGAAGAGGAGGAAGGGAGGCCAGGGCCGGCGGGAGAAT
GCCAACAGGAACCTGGCCAGGAAGGAGAGCAAGGAGGCGGGTGCTGGCTCTCGAAGACGCAAGGGGCAGC
AACAGCAGCAGCAGCAAGGGACAGTGGGGCCACTCACATCTGCAGGGCCTGCCTAGGGACACTGTCCAGC
CTCCAGGCCCATGCAGAAAGAGTTCAGTGCTACTCTGCGTGATTCAAGCTTTCCTGAACTGGAACGTCGG
GGGCAAAGCATACACACACACTCCAATCCATCCATGCATACATAGACACAAGACACACACGCTCAAACCC
CTGTCCACATATACAACCATACATACTTGCACATGTGTGTTCATGTACACACGCAGACACAGACACCACA
CACACACATACACACACACACACACACACACCTGAGGCCACCAGAAGACACTTCCATCCCTCGGGCCCAG
CAGTACACACTTGGTTTCCAGAGCTCCCAGTGGACATGTCAGAGACAACACTTCCCAGCATCTGAGACCA
AACTGCAGAGGGGAGCCTTCTGGAGAAGCTGCTGGGATCGGACCAGCCACTGTGGCAGATGGGAGCCAAG
CTTGAGGACTGCTGGTGACCTGGGAAGAAACCTTCTTCCCATCCTGTTCAGCACTCCCAGCTGTGTGACT
TTATCGTTGGAGAGTATTGTTACCCTTCCAGGATACATATCAGGGTTAACCTGACTTTGAAAACTGCTTA
AAGGTTTATTTCAAATTAAAACAAAAAAATCAACGACAGCAGTAGACACAGGCACCACATTCCTTTGCAG
GGTGTGAGGGTTTGGCGAGGTATGCGTAGGAGCAAGAAGGGACAGGGAATTTCAAGAGACCCCAAATAGC
CTGCTCAGTAGAGGGTCATGCAGACAAGGAAGAAAACTTAGGGGCTGCTCTGACGGTGGTAAACAGGCTG
TCTATATCCTTGTTACTCAGAGCATGGCCCGGCAGCAGTGTTGTCACAGGGCAGCTTGTTAGGAATGAGA
ATCTCAGGTCTCATTCCAGACCTGGTGAGCCAGAGTCTAAATTTTAAGATTCCTGATGATTGGCATGTTA
CCCAAATTTGAGAAGTGCTGCTGTAATTCCCCTTAAAGGACGGGAGAAAGGGCCCCGGCCATCTTGCAGC
AGGAGGGATTCTGGTCAGCTATAAAGGAGGACTTTCCATCTGGGAGAGGCAGAATCTATATACTGAAGGG
CTAGTGGCACTGCCAGGGGAAGGGAGTGCGTAGGCTTCCAGTGATGGTTGGGGACAATCCTGCCCAAAGG
CAGGGCAGTGGATGGAATAACTCCTTGTGGCATTCTGAAGTGTGTGCCAGGCTCTGGACTAGGTGCTAGG
TTTCCAGGGAGGAGCCAAACACGGGCCTTGCTCTTGTGGAGCTTAGAGGTTGGTGGGGAAGAAAATAGGC
ATGCACCAAGGAATTGTACAAACACATATATAACTACAAAAGGATGGTGCCAAGGGCAGGTGACCACTGG
CATCTATGCTTAGCTATGAAAGTGAATAAAGCAGAATAAAAATAAAATACTTTCTCTCAGG
Mus musculus R-spondin 1 (Rspo1), mRNA
NCBI Reference Sequence: NM_138683.2
>NM_138683.2 Mus musculus R-spondin 1 (Rspo1), mRNA
SEQ ID NO: 12
GGATTCCCTCCCTCGTGCGAGCCGGGGACCGGCCCCTCTCCGGGCGCGGGGCGCAGAGCCCGGGCGGCGC
ACTGCGGGGCCCGCGCGGGCCGCCCCAGCACCAATGCACCGGGCGGGGCGCTGGCGGCCGAGAAGGCATT
GAGCAACTGGGCGGCGGGCGGAGCGCGGGGCCGACGGCAACGCGGGACCCAGTGGCCGCGCCCTCGCCCC
TCCGGGCTGCCCCGCCACGGCCGCTGCGCCAGGTCTATCTTGGGGGTGGTTCTCTGCTGGCGTGAGAAGA
CTTCTCATGTGACCCTCTGAGGTGGATTCAAGCAGGACAGGACCTCCCTTTGGACCAATGGAGAAGCCGG
CTCCAAACCCTCTCGGATCCCAGCTAAGGTTATGGTGGATCCGGGCCTGGCTCTCCTGCCACTGACCCAG
CCTCAGAGCCTTTTAGCAAGAGACCACCCCTCCTGCCAGGGGCCCGGGGCTGGCCAGTGACTATGCGGCT
TGGGCTGTGCGTGGTGGCCCTGGTTCTGAGCTGGACACACATCGCCGTGGGCAGCCGGGGGATCAAGGGC
AAGAGACAGAGGCGGATCAGTGCTGAGGGGAGCCAAGCCTGCGCCAAGGGCTGTGAGCTCTGTTCAGAAG
TCAACGGTTGCCTCAAGTGCTCGCCCAAGCTCTTCATTCTGCTGGAGAGGAACGACATCCGCCAGGTGGG
CGTCTGCCTGCCGTCCTGCCCACCTGGATACTTTGATGCCCGCAACCCCGACATGAACAAATGCATCAAA
TGCAAGATCGAGCACTGTGAGGCCTGCTTCAGCCACAACTTCTGCACCAAGTGTCAGGAGGGCTTGTACT
TACACAAGGGCCGCTGCTATCCAGCCTGCCCTGAGGGCTCTACAGCCGCTAACAGCACCATGGAGTGCGG
CAGTCCTGCACAATGTGAAATGAGCGAGTGGTCCCCGTGGGGACCCTGCTCCAAGAAGAGGAAGCTGTGC
GGTTTCCGGAAGGGATCGGAAGAGCGGACACGCAGAGTGCTCCATGCTCCCGGGGGAGACCACACCACCT
GCTCCGACACCAAAGAGACCCGCAAGTGTACCGTGCGCAGGACGCCCTGCCCAGAGGGGCAGAAGAGGAG
GAAGGGGGGCCAGGGCCGGAGGGAGAATGCCAACAGGCATCCGGCCAGGAAGAACAGCAAGGAGCCGGGC
TCCAACTCTCGGAGACACAAAGGGCAACAGCAGCCACAGCCAGGGACAACAGGGCCACTCACATCAGTAG
GACCTACCTGGGCACAGTGACCGGTCTCCAGATACCTGTGGAAGAGTACAGTGCTGTACTGTATAATGAG
AACTTTCCAGAACTGGAGCATCTGGGAGAGTCCACACATACCCCATCCACCCACCCATCCAACTATCCAT
CCATCCATCCATGCACACATATGGCCACATCTGAAAACGTCAACACACACACACACACACACACACACAC
ACACACACATTCTTGAGGTCACTGAAGACACTTCTATTCTGTGGCCCAGCTGTATATTCAGTCTTTAATG
CTCTTGGAAGACATATCTGAGAGAACCTTTCCCAGCATCTGAAACTAAGGAGTGGAACCTTCTGGAGGAA
CTTCTGGGACAGCATCTGACAGATGGATGGCAGATTGGAGCCAAAGCTGGAGCAGCTGCCGAGAGGGAGA
GAGAGGGAAAGCGCTTTCCCGGCTTGAGAGGCACTCCCAGCTGTGAGACTTGATTGTCGGAGATGAGAAT
TATTACACATCCGTGGTACACGTCACGGATGACCTGACTTGGAAACTGCTTAAAGGTTTATTTCAAATTA
AAAAAGAGAAAAAC
R-spondin-1 isoform 1 precursor [Homo sapiens] NCBI Reference
Sequence: NP_001033722.1
>NP_001033722.1 R-spondin-1 isoform 1 precursor [Homo sapiens]
SEQ ID NO: 15
MRLGLCVVALVLSWTHLTISSRGIKGKRQRRISAEGSQACAKGCELCSEVNGCLKCSPKLFILLERNDIR
QVGVCLPSCPPGYFDARNPDMNKCIKCKIEHCEACFSHNFCTKCKEGLYLHKGRCYPACPEGSSAANGTM
ECSSPAQCEMSEWSPWGPCSKKQQLCGFRRGSEERTRRVLHAPVGDHAACSDTKETRRCTVRRVPCPEGQ
KRRKGGQGRRENANRNLARKESKEAGAGSRRRKGQQQQQQQGTVGPLTSAGPA
R-spondin-1 precursor [Mus musculus]
NCBI Reference Sequence: NP_619624.2
>NP_619624.2 R-spondin-1 precursor [Mus musculus]
SEQ ID NO: 16
MRLGLCVVALVLSWTHIAVGSRGIKGKRQRRISAEGSQACAKGCELCSEVNGCLKCSPKLFILLERNDIR
QVGVCLPSCPPGYFDARNPDMNKCIKCKIEHCEACFSHNFCTKCQEGLYLHKGRCYPACPEGSTAANSTM
ECGSPAQCEMSEWSPWGPCSKKRKLCGFRKGSEERTRRVLHAPGGDHTTCSDTKETRKCTVRRTPCPEGQ
KRRKGGQGRRENANRHPARKNSKEPGSNSRRHKGQQQPQPGTTGPLTSVGPTWAQ

As used herein, the term “sensitivity” is the percentage of subjects with a particular disease.

As used herein, the term “specificity” is the percentage of subjects correctly identified as having a particular disease i.e., normal or healthy subjects. For example, the specificity is calculated as the number of subjects with a particular disease as compared to non-cancer subjects (e.g., normal healthy subjects).

The term “specifically binds” refers to a compound or antibody that recognizes and binds a polypeptide or nucleic acid of the invention, but which does not substantially recognize and bind other molecules in a sample, for example, a biological sample, which naturally includes a polypeptide of the invention.

As used herein, the term “subject” is intended to refer to any individual or patient to which the method described herein is performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof Δny compositions or methods provided herein can be combined with one or more of any of the other compositions and methods provided herein.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

Such treatment (surgery and/or chemotherapy) will be suitably administered to subjects, particularly humans, suffering from, having, susceptible to, or at risk for pancreatic cancer or disease, disorder, or symptom thereof. Determination of those subjects “at risk” can be made by any objective or subjective determination by a diagnostic test or opinion of a subject or health care provider (e.g., genetic test, enzyme or protein marker, a marker (as defined herein), family history, and the like).

### BRIEF DESCRIPTION OF THE DRAWINGS

FIG. 1. Generation of GLT1 promoter reporter mice employing increasing lengths of the DNA sequence upstream of the transcription start site. Promoter reporter mice were generated by employing different lengths of the DNA sequences upstream of the transcriptional start site (TSS) of GLT1 followed by tdTomato which were inserted into constructs for pronuclear injection to generate transgenic mice, in two to three different founder lines leading to astroglia-specific tdTomato-expression (see details below). (A) Varying length promoter reporter fragments resulted in differential cell-specific tdTomato expression. A fragment of 8.3 kb was required for astroglia-restricted tdTomato expression, whereas fragments ≤7.9 kb resulted in neuronal tdTomato expression and no astroglial expression in adults. (B) Coronal section of the 8.3 kb-tdTomato expression. (C) Magnified image of motor cortex representing overlap between 8.3 kb-tdTomato and Glt1-eGFP. (D) No 8.3 kb-tdTomato expression was detected in the hippocampus. A minimum of 3 mice were imaged with analysis of 3-5 images per mouse.

FIG. 2. 8.3 kb-tdTomato expression is static and limited to an astroglia subset in the cerebral motor cortex. (A) 8.3 kb-tdTomato does not colocalize with neuronal marker, NeuN. (B) 8.3 kb-tdTomato does not colocalize with microglia marker, Ibal. (C) 8.3 kb-tdTomato does not colocalize with myelin and oligodendrocyte marker, CNPase. (D) 8.3-astroglia consist of about 25% of all grey matter astroglia in the motor cortex. (E) 8.3-astroglia are heavily enriched in cortical layers II/III and V. Glt1-eGFP only astroglia are equally distributed across all cortical layers. (F) 8.3 kb-tdTomato nanoparticles are expressed by a subset of GFAP-positive astroglia enriched in layers II/III and V of the mouse motor cortex. (G) Cortical multiphoton in vivo imaging performed weekly for 5 weeks in adult mice tracking individual 8.3-astroglia (N=5 mice, 100 cells). Astroglia cell counting was performed with sections imaged and subjected to validated automated cell counting of eGFP and tdTomato fluorescence cells (CITE). For nanoparticle injections, at 5 mice were intracortically injected and analyzed. For all experiments a minimum of 5 mice were analyzed, 3-5 images per mouse.

FIG. 3. Astroglial cell populations isolated from 8.3-astroglia mouse CNS and human cortex have unique transcriptomes as validated with immunohistochemistry (IHC) and RNA in situ hybridization. (A) The cortex microarray heatmap displays unique genes differentially expressed between all rodent astroglia (GLT1-eGFP), 8.3 kb labeled astroglia (8.3-astroglia) and remaining non-labeled cells (negative). (B) Microarray expression of astroglia markers highly expressed in both rodent astroglia populations. (C) Selected markers highly enriched in 8.3-astroglia microarray data in the cortex. (D) qPCR validation of candidate markers from the microarray analyses in the cortex of the three cell populations. (E) KCNJ10 enhanced immunoreactivity in 8.3-astroglia compared to GLT1-eGFP astroglia in the mouse motor cortex. (F) LGR6-GFP expression is restricted to 8.3-astroglia in the adult mouse cortex as evaluated in double transgenic LGR6-GFP-ires-CreERT2/8.3-astroglia mice. (G) Analysis of Lgr6 mRNA expression in the human motor cortex in a subset of cells. (H) LGR6 protein in the human motor cortex colocalizes with GFAP and ALDH1L1 protein. (I) A subset of cultured primary mouse cortical astroglia are LGR6-positive. (J) A subset of cultured and matured human iPSC-astroglia are LGR6-immunoreactive. Three cell populations were isolated from the adult mouse whole cortex and subjected to FACS, microarray, and qPCR analyses from a total of three mice. For histological sections, at least five mice were imaged with a minimum of 3-5 images per mouse, representative images are shown. Human post-mortem tissue was evaluated in three non-neurological diseased cases with at least 3-5 images taken per case for both RNA ISH and immunohistochemistry. For mouse immunocytochemistry, at least five different cultures of mouse primary astroglia were generated and imaged at least 3-5 times per astroglia generation. For iPSC astroglia a minimum of three control human iPSC lines were generated. All images are representative of the total amount imaged. Statistics were performed by Student's T-test of 8.3-astroglia vs negative cell population, *p<0.05, **p<0.01, ***p<0.001.

FIG. 4. Functional and dysfunctional properties of astroglia subpopulation by examination of LGR6 pathways in the cerebral motor cortex. (A) Neuron-specific RSPO1 immunoreactive localization is restricted to cortical layer V in the mouse motor cortex (c.c.=corpus callosum). (B) RSPO1 treatment in vitro of primary mouse astroglia increases LGR6 immunoreactivity. (C) Lgr6-heterozygote mice display a reduction in the numbers of 8.3-astroglia. (D) Partial loss of LGR6 alters astroglial cortical density of 8.3 astroglia as Lgr6-heterozygote mice display a significant reduction in numbers of 8.3-astroglia. (E) LGR6 alters neurite properties as Lgr6-heterozygote null mice have reduced spine density compared to control mice as assessed with the Golgi-Cox staining. (F) LGR6 receptor agonist RSPO1 affects astroglial Norrin. Norrin levels are increased following RSPO1 treatments on primary astroglia as assessed by ELISA. (G) Norrin mRNA colocalizes with 8.3-astroglia in the mouse motor cortex. At least five different mice with 3-5 coronal slices were imaged for IHC and RNA FISH. Five mice were used for spine analysis with 5 neurons analyzed per mouse motor cortex. At least three different primary astroglia cultures were treated for 48 hours with RSPO1 and repeated 3-5 times for ELISA analysis. Statistics performed was Student's T-test, *p<0.05, **p<0.01, ***p<0.001.

FIG. 5. Norrin is a neuro-modulating protein that affects cortical neuron dendritic morphology and spine density. (A) Norrin-treated mouse cortical neurons show increased length and branching. (B) Treatment with Norrin lead to a significant number of neurite changes including intersections and neurite branching as revealed by Scholl analytics. (C) Neurite length is increased after Norrin treatment of cultured cortical mouse neurons. (D) Loss of Norrin in vivo leads to a significant defect in cortical neuron spine density. Layer V cortical neurons were identified in adult Norrin-null mouse motor cortex and dendritic spines were examined after Golgi-Cox staining. (E) Norrin-null mice have significantly reduced spine density compared to wild-type. (F) Intracortical nanoparticle injections containing 8.3 kb-Norrin significantly increase dendritic spine density in cortical layer V of the mouse motor cortex. Statistics performed by Student's T-test of comparing Norrin treated vs control, *p<0.05, **p<0.01, ***p<0.001, ****p<0.0001. At least 5 different treatments of recombinant Norrin were treated on 10 different isolations of cortical neurons with a minimum of 20 neurons analyzed for each cohort for 48 hours. For spine density analysis, at least five P60 mice were used using Golgi-Cox staining kit; 5-10 cortical neurons, located in the motor cortex cortical layer V, were counted and analyzed per genotype, representative images are shown.

FIG. 6. Norrin alters the electrophysiological properties of cortical neurons and Norrin-null mice display neurobehavioral abnormalities. (A) Norrin-treatment enhances cortical neuron connectivity and firing rate. (B) Norrin-treatment significantly increases the degree of neuronal firing. (C) Norrin-treatment significantly increases the weight of the neuronal firing strength. (D) Norrin-null mice are significantly more hyperactive than their wildtype littermate controls. At least 3 different treatments were analyzed for the MEA. For the open field assay 15-20 mice were used per genotype. Statistics include 2-way Repeated ANOVA, and Student T-test, *p<0.05, **p<0.01, ***p<0.001, ****p<0.0001.

FIG. 7. Selection and generation of the Glt1 promoter fragment transgenic mice. (A) Glt1 (human nomenclature EAAT2) shows four highly methylated sites in its hypothesized promoter region, which were selected for the generation of the transgenic mice.

FIG. 8. Promoter fragments <8.3 kb result in tdTomato in non-astroglia cells in the cerebral cortex. (A) 2.5 kb and 7.9 kb appear to label neurons in the cerebral cortex. (B) The fragment sizes 2 kb, 6.7 kb, and 7.9 kb result in no colocalization with astroglia marker Glt1 but colocalize with neuronal marker, NeuN. At least two to three mice were imaged with 3-5 images per mouse.

FIG. 9. Additional approaches to validate astroglia TdTomato expression and to validate 8.3 promoter construct using nanoparticles. (A) CLARITY-optimized light-sheet microscopy (COLM) imaging was employed to visualize in 3D the whole brain distribution of the 8.3-astroglia. COLM shows the restricted pattern of expression of the 8.3-astroglia in the cortex as well paucity of expression in deep telencephalon structures. (B) Glt1-eGFP colocalizes with 8.3 kb-tdTomato. (C,D) Nanoparticles were packaged and injected intracortically. 8.3 kb-tdTomato expression is observed in cortical layers II/III and V, consistent with the transgenic mice. (E,F) Nanoparticles were packaged with CMV-eGFP and the 8.3 kb constructs as an additional tool to evaluate astroglial specific TdTomato expression. Transmission electron microscopy at 80,000× magnification was used to show the diameter of CMV-GFP nanoparticles. Transmission electron microscopy at 80,000× magnification was used to show the diameter of 8.3 kb-tdTomato nanoparticles. At least two mice were used for COLM. At least three mice were used for ex vivo histology with 3-5 images analyzed per mouse, representative images are shown.

FIG. 10. 8.3-astroglia maintain stable tdTomato expression in vitro and have a unique transcriptome. (A) FACS gating for isolation of CNS cells. FACS was performed on cells isolated from adult dissociated adult cortex of BAC-Glt1-eGFP/8.3-astroglia mice (n=3) and shows reliably identified three fluorescently-unique cell populations: eGFP-only, TdTomato/eGFP, and negative. (B) 8.3-astroglia maintain stable tdTomato-expression in vitro. (C) GLT1-eGFP-only astroglia do not express tdTomato in vitro. (D) The top Ingenuity canonical pathways are listed for each astroglia population and for pathways shared between both astroglia populations from the microarray data. (E) OLIG2 colocalizes with 8.3-astroglia in the motor cortex. The collected populations were subjected to microarray RNA and qPCR analyses (n=3 mice). Cells isolated by dissociation into single-cell and maintained in astroglia medium without FBS for up to two weeks before passaging. For histology, at least three mice were used with 3-5 images per mouse analyzed.

FIG. 11. 8.3-astroglia are LGR6 and KCNJ10 positive. (A) LGR6 colocalizes with ALDHIL1 in adult mouse motor cortex. (B) KCNJ10 is enriched in 8.3-astroglia. (C) KCJN10 mean intensity fluorescence is increased significantly in 8.3-astroglia vs Glt1-eGFP astroglia (N=100 cells). At least 3-5 mice were imaged with 3-5 images per mouse analyzed. Statistics include Student T-test, *p<0.05, **p<0.01, ***p<0.001, ****p<0.0001.

FIG. 12. Rspo1 is restricted to a neuronal subset in the lower cortical layers in the motor cortex. (A) Rspo1-positive mRNA is identified in a cell in the lower cortical layers. (B) Rspo1 is not observed in all cells in lower cortical layers. (C) Rspo1 is restricted to neurons in the lower cortical layers of the motor cortex. (D) Rspo1 is in not observed in all neurons in the lower cortical layers of the motor cortex. At least 3-5 mice were analyzed with 3-5 images per mouse.

FIG. 13. RSPO1-treatment increases LGR6-positive astroglia proliferation but in vivo knockdown of LGR6 leads to reduced cortical thickness. (A) Dose-dependent increase in astroglia post 24-hour RSPO1-treatment. (B) Dose-dependent increase in the overall amount of LGR6-positive astroglia post 24-hour RSPO1-treatment. (C) Lgr6-heterozygote null mice display a significant reduction in cortical thickness. At least 5 different primary astroglia isolations with 3-5 repeats per treatment were used for RSPO1 treatments. At least seven mice were used with 3-5 images per mouse for cortical thickness. Thickness was calculated by measuring the distance from the corpus collasum to the pia of the motor cortex following the Allen Brain Mouse Atlas Reference. Statistics performed was Student's T-test between experimental and control, *p<0.05, **p<0.01, ***p<0.001.

FIG. 14. Norrin-treatment affects the overall morphology of cortical neurons. (A) Primary cortical neurons treated with a PBS vehicle control. (B) Primary cortical neurons treated with Norrin for 48 hours show increased branching and dendritic length compared to the PBS control. At least 5-10 primary cortical isolations with 3-5 repeats per treatment and 15-20 neurons analyzed per treatment. Somas are drawn in, images not to scale.

FIG. 15. Norrin truncated proteins effect on primary cortical neurons. (A) Primary cortical neurons treated for 48 hours with truncated protein 1. (B) Primary cortical neurons treated for 28 hours with truncated protein 2. At least 5-10 primary cortical isolations with 3-5 repeats per treatment and 15-20 neurons analyzed per treatment. Somas are drawn in, images not to scale.

FIG. 16. Lgr6-heterozygote and Norrin-null mice exhibit spine density deficits in cortical layer V of the motor cortex. (A) Wld-type mice dendritic spines. (B) Lgr6-heterozygote spine density is reduced compared to wild-type. Five mice were analyzed per genotype for spine density. (C) Norrin-null spine density is reduced compared to wild-type. Representative images are shown from apical dendrites from pyramidal layer V neurons, a minimum of 5 neurons analyzed per mouse.

FIG. 17. Norrin-treatment alters the electrophysiology of cortical neurons. (A) Rostaplots of the MEA recordings after 24 hours of treatment with Norrin. Representative images are shown.

FIG. 18. Norrin-treatment alters the electrophysiology of cortical neurons. (A) Number of spikes is significantly increased post Norrin-treatment. (B) Percent of spikes in a burst on the MEA recordings is significantly increased post Norrin-treatment. (C) Burst total weight is significantly increased in Norrin treated neurons. (D) Degree of bursts is significantly increased post Norrin-treatment. (E) Degree of spikes is significantly increased in Norrin treated neurons. (F) Weight total of the neuronal spikes is significantly increased in Norrin treated neurons. Repeated at least three times. Statistics performed was Student's T-test between experimental and control, *p<0.05, **p<0.01, ***p<0.001.

FIG. 19. Norrin-null mice display neurobehavioral abnormalities consistent with a hyperactive phenotype. (A) Norrin-null mice enter Y-maze arms significantly more than wildtype littermates. (B) Norrin-null mice are significantly faster. (C) Norrin-null mice travel significantly more than wildtype littermates. (D) Norrin-null mice rest significantly less than their wildtype littermates. (E) Norrin-null mice display significantly more rearing than their wildtype littermates. A minimum of 12 mice were used for behavioral assays. Results were analyzed blinded. Statistics performed was Student's T-test between experimental and control, *p<0.05, **p<0.01, ***p<0.001.

FIG. 20. The physiological pathway hypothesized for 8.3-astroglia interacting with corresponding neuronal neighbors. (A) 8.3-astroglia residing the adult motor cortex typically display normal levels of Lgr6 and Norrin, which lead to typical neuronal morphology, electrophysiology, and overall behavior. Deletion or knockdown of LGR6 or Norrin leads to altered dendritic morphology, spine density, and behavior.

FIG. 21: 8.3-astroglia/SOD1 G93A mouse motor cortex and lumbar spinal cord at end-stage P120. A) Control 8.3-astroglia vs SOD1 G93A end-stage. B) Kir4.1 immunofluorescence in control 8.3-astroglia vs SOD1 G93A end-stage. C) Kir4.1 immunofluorescence in the lumbar spinal cord of control 8.3-astroglia vs SOD1 G93A end-stage. D) 8.3-astroglia in the lumbar spinal cord of control 8.3-astroglia vs SOD1 G93A end-stage. A minimum of 5 mice were analyzed, with 3-5 images analyzed per mouse.

FIG. 22: Human post-mortem motor cortex of control vs c9orf72 ALS. A) Control vs ALS patient motor cortex with Lgr6-immunofluorescence. B) Quantification of Lgr6-positive astroglia in the human control vs ALS motor cortex. C) Relative mRNA levels of Lgr6 in the human motor cortex of control vs ALS. D) Quantification of Lgr6 protein levels detected from western blot for human control vs ALS motor cortex. A minimum of 4-5 patients were analyzed, with 3-5 images analyzed per patient.

FIG. 23: hiPSC-astroglia from control and ALS astrocyte iPS lines. A) Lgr6-positive astroglia in control, SOD1, and c9orf72 patient hiPSC-astroglia. B) Kir4.1-immunoreactivity in control, SOD1, and c9orf72 patient hiPSC-astroglia. C) Quantification of number of Lgr6-positive astroglia. D) Quantification of the overall mean intensity fluorescence of Kir4.1 in patient human iPSC-astroglia. At least 3-5 patient lines were differentiated and analyzed.

FIG. 24: Molecular and cellular functions in 8.3-astroglia from microarray analysis of neocortex Top differentially expressed molecular and cellular functions determined by IPA using the list generated from the microarray analysis of the tdTom+astroglia as compared to eGFP+astroglia and the negative population. Bold=upregulated; Italicized=downregulated

FIG. 25: Microarray analysis of 8.3-astroglia/BAC-GLT1-eGFP astroglia from mouse neocortex. List of some of the top differentially expressed candidates for the identification of the astrocyte subpopulation based on microarray analysis of the neocortex. High stringency was performed for this analysis, including a p-value <0.1 and absolute fold change >1.5, resulting in 260 candidates. Bold=upregulated; Italicized=downregulated

FIG. 26: Ingenuity Pathway Analysis derived from microarray analysis of 8.3-astroglia/BAC-GLT1-eGFP astroglia from mouse neocortex. Top IPA expressed canonical pathways in tdTom-positive astroglia as compared to the eGFP-positive only astroglia and negative population. Analysis performed using same stringency as FIG. 28. Bold=upregulated; Italicized=downregulated

FIG. 27: Astrocyte-enriched candidates from microarray analysis of 8.3-astroglia/BAC-GLT1-eGFP astroglia from mouse neocortex. List of selected enriched candidates shared among astrocyte populations based on microarray analysis of the neocortex. High stringency was performed for this analysis, including a p-value <0.05 and absolute fold change >2.5 against the negative cell population, and absolute fold change no greater than 2.0 between GFP-positive/tdTomato-positive and GFP-positive/negative cells, resulting in 3700 candidates. Bold=upregulated; Italicized=downregulated

FIG. 28: Table S5. Patient Demographics. Human Cortex used for Histology

### DETAILED DESCRIPTION OF THE INVENTION

To begin to develop insight into possible astroglia subgroups, the inventors explored the biology of an astroglia-specific synaptic protein, the glutamate transporter EAAT2, known to be focally altered in some paradigms.

All astroglia normally express EAAT2, also known as GLT1. Astroglial GLT1 is generally expressed uniformly throughout the CNS, although the levels of GLT1 are approximately 10-fold higher in the hippocampus and cortex relative to the spinal cord. Dramatic regional dysregulation of GLT1 expression has been observed in a variety of neurological and psychiatric disorders. For example, GLT1 is downregulated in a subset of astroglia in the ventral spinal cord and layers of motor cortex in human ALS patients as well as in rodent models of ALS. Although the biological basis of this focal dysregulation has historically focused on how neurons may focally regulate GLT1 expression, we sought to define the regulation of GLT1 expression within astroglia specifically.

Results

Identification and Validation of 8.3-Astroglia: Glt1 Promoter Reporter Mouse Lines

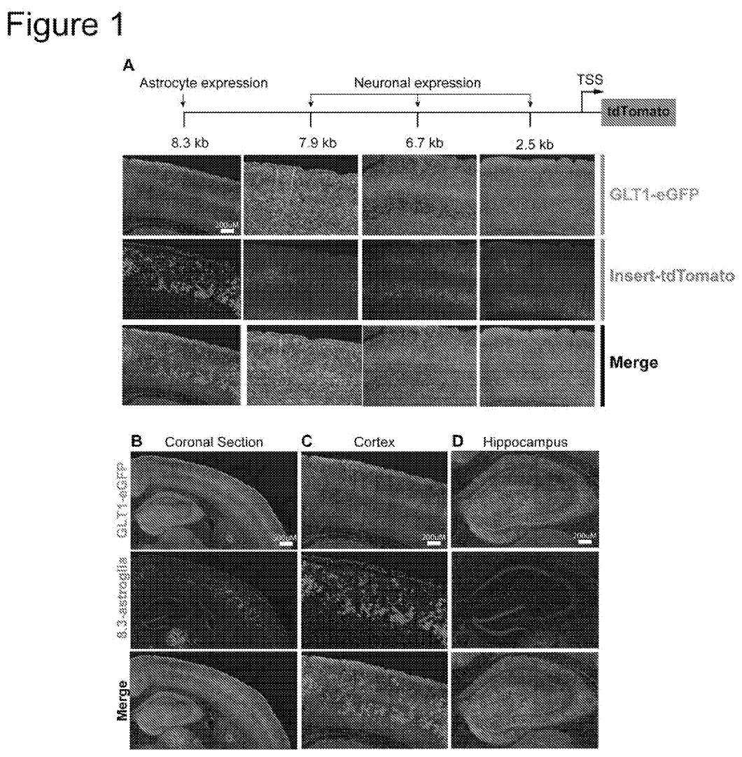

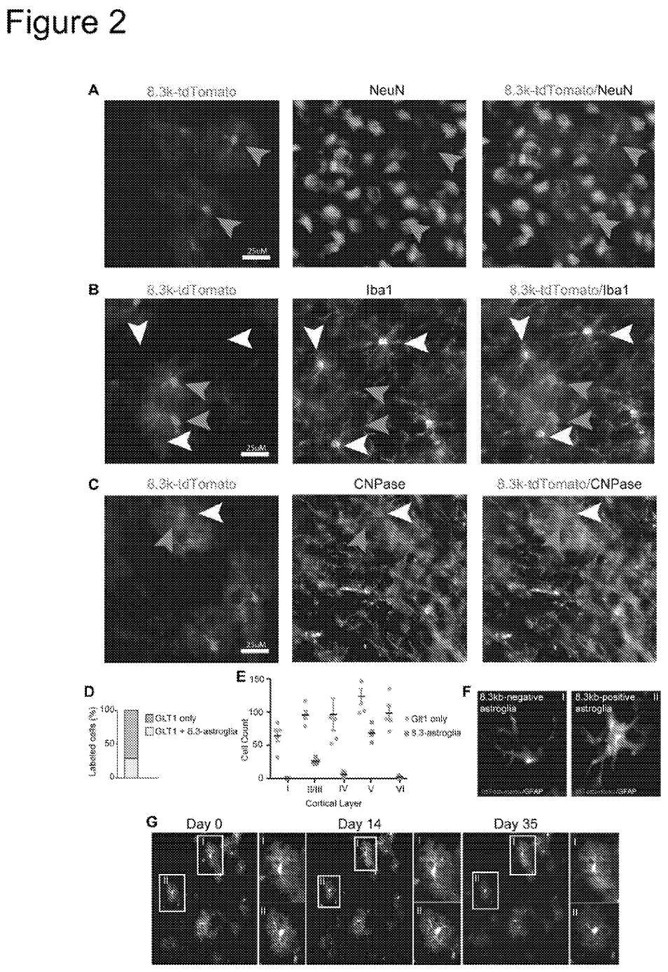

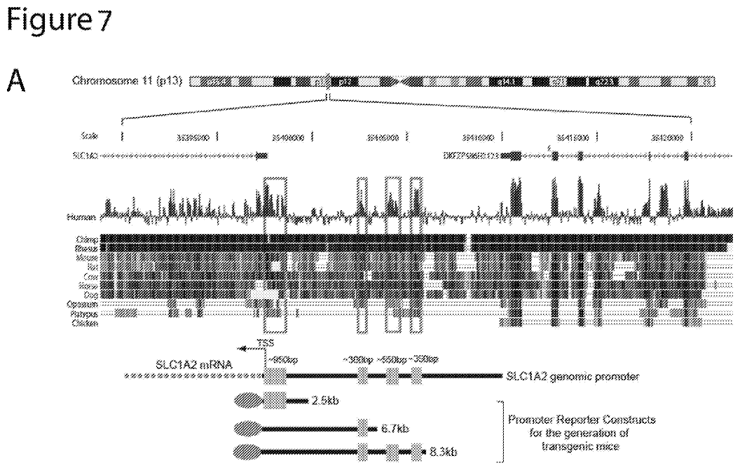

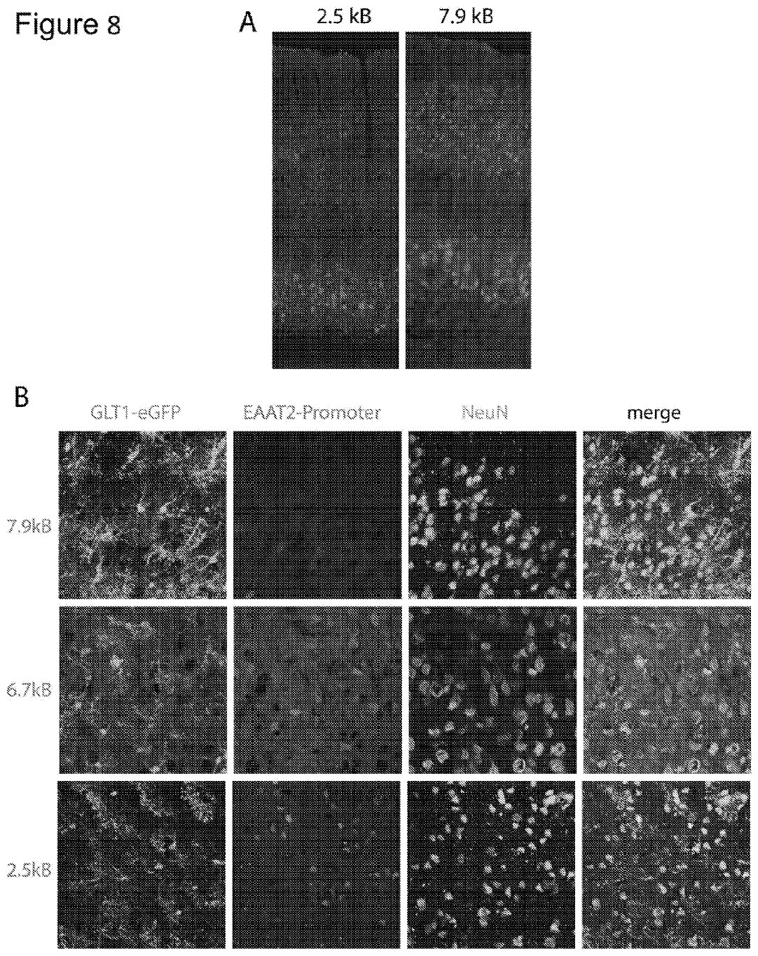

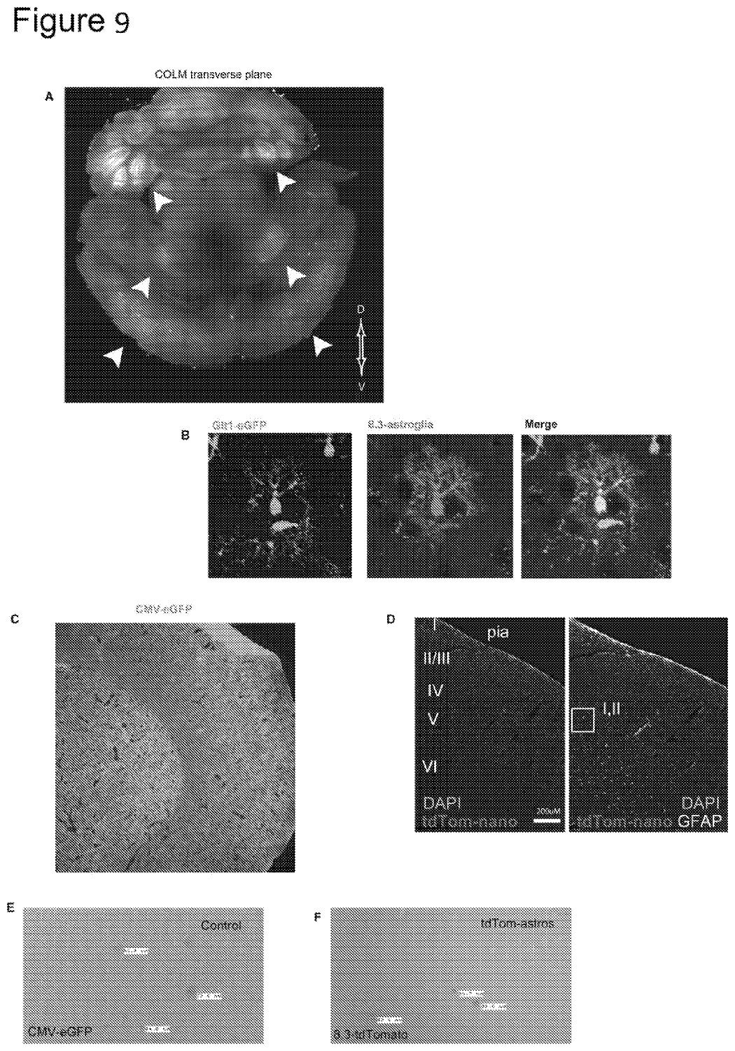

To examine Gill regulation within astroglia, the inventors created multiple mouse lines using DNA inserts that contained fragments of the Gill promoter upstream of the Gill transcriptional start site. Downstream of the Glt1 transcriptional start site, the insertion of a tdTomato reporter allowed us to visualize cell populations actively transcribing the transgenic constructs. The fragments of the Gill promoter were of various sizes, ranging from 2.5 kb to 8.3 kb upstream of the transcription start site (FIG. 1A, FIG. 7A). These fragment sizes were chosen based on conserved genomic regions with high methylation between the mouse and human Glt1gene (human nomenclature, EAAT2) (FIG. 7A). Multiple founder lines expressing a promoter fragment ≤7.9 kb showed tdTomato expression that was restricted to neurons (FIG. 1A, FIG. 8A-B). Unexpectedly, founder lines with only a slightly larger insert size, 8.3 kb, showed tdTomato fluorescence limited to grey-matter astroglia (FIG. 1A to C, FIG. 2A-C, FIG. 9B). Notably, a specific and reproducible subpopulation of grey-matter astroglia expressed tdTomato in the cerebral cortex and several other regions unexplored in this study (FIG. 1B-D, FIG. 9A). To compare this tdTomato-astroglia population (henceforth referred to as “8.3-astroglia”) with all other grey-matter cortical astroglia, we crossed 8.3-astroglia-labeled mice with a BAC-GLT1-eGFP mouse line (GLT1-eGFP), which labels all CNS astroglia with eGFP (FIG. 1A-C, FIG. 9B). In the double transgenic mouse line (8.3-astroglia/GLT1-eGFP), all 8.3-astroglia were also eGFP-positive, indicating that these cells are indeed astroglia expressing GLT1 (FIG. 2A-C, FIG. 9B). In adult cortex grey matter, definable subsets of all eGFP-astroglia were also tdTomato-positive (FIG. 1B-C, FIG. 9B). Notably, 8.3-astroglia were completely absent from the hippocampus (FIG. 1D).

The 8.3-astroglia consisted of approximately 28% of all cortical astroglia (FIG. 2D) as evaluated by the number of 8.3-astroglia/GLT1-eGFP double-positive astroglia in the adult cortex. Layer V of the cortex was preferentially enriched with 8.3-astroglia compared to all other cortical layers, whereas the GLT1-eGFP single-positive astroglia were evenly distributed among the cortical layers as assessed by traditional 2D as well as 3D imaging approaches (FIG. 2E).

To verify that these observations were not due to an artifact of genomic integration and to provide an alternative approach for studying this astroglia subset without the need for labeled transgenic mice, we introduced the 8.3 kb-tdTomato plasmid in vivo in wild-type mice using nanoparticles. Nanoparticles have the advantage of accepting large molecular constructs and have excellent in vivo tissue distribution properties compared to adeno-associated viruses or lentiviruses. CMV-eGFP control and 8.3 kb-tdTomato plasmids were packaged into separate nanoparticles that were approximately the size of nanometer-sized exosomes as visualized by transmission electron microscopy (FIG. 9E-F). Following intracortical injection of CMV-eGFP control nanoparticles, eGFP fluorescence was distributed across the entire hemisphere and in all cell types (FIG. 9C). After cortical injection of the 8.3 kb-tdTomato nanoparticles, however, tdTomato expression was limited to astroglia in the grey matter and specifically enriched in layers II/III and V, consistent with the 8.3-astroglia transgenic mouse (FIG. 2F, 9D). These data support the hypothesis that the 8.3 kb sequence upstream of the Gill transcriptional start site is utilized only by a subset of astroglia.

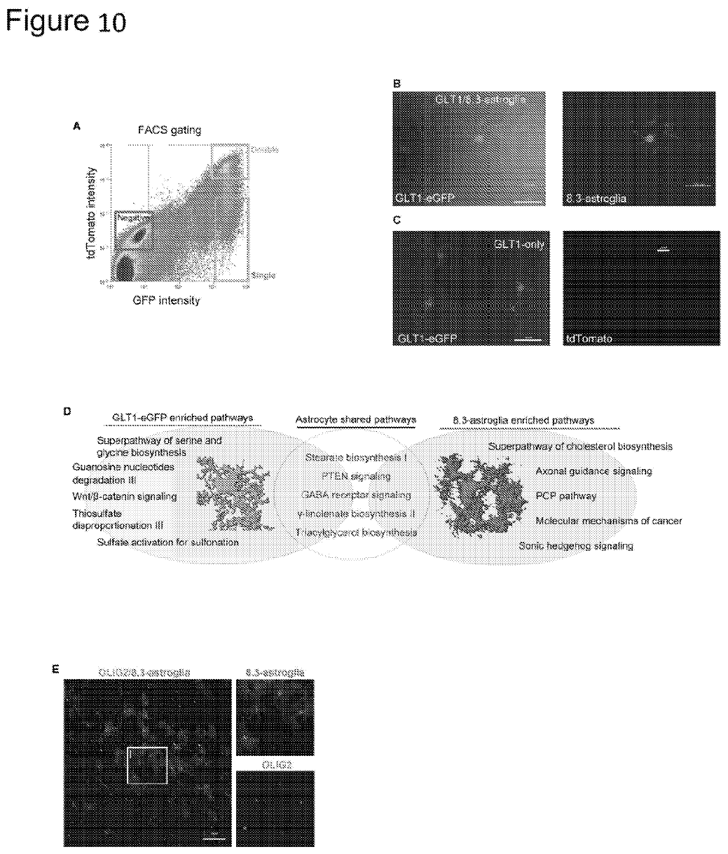

The inventors next determined the stability of tdTomato fluorescence in this astroglia subset. Stable expression of tdTomato within a subset of astroglia throughout their lifespan, without the appearance of additional fluorescent signal from other cell populations, would support the use of tdTomato as a fluorescent marker to study the specific subset of cells utilizing the 8.3 kb promoter fragment. To address this possibility, the inventors performed longitudinal multiphoton imaging of the motor cortex (up to 500 μm deep) of 8.3-astroglia-labeled transgenic mice. The inventors tracked individual 8.3-astroglia over a period of five weeks and saw no changes in fluorescence, nor did they observe the appearance of any additional tdTomato-expressing cells in the cortical regions studied (FIG. 2G). The cell-specific tdTomato fluorescence was first observed during developmental astrogenesis (P5) and remained stable throughout adulthood (not shown). Finally, the inventors assessed the longitudinal expression of tdTomato in vitro using isolated 8.3-astroglia from adult mouse cortex (P60-P90), finding that tdTomato fluorescence continued for up to two weeks prior to cell passaging (FIG. 10B—C). The continuous expression of tdTomato in 8.3-astroglia in vivo and in vitro supports the hypothesis that these cells represent a distinct subpopulation of astroglia and that this transgenic mouse model provides a tool to study this cell population in various contexts.

Molecular Properties of 8.3-Astroglia

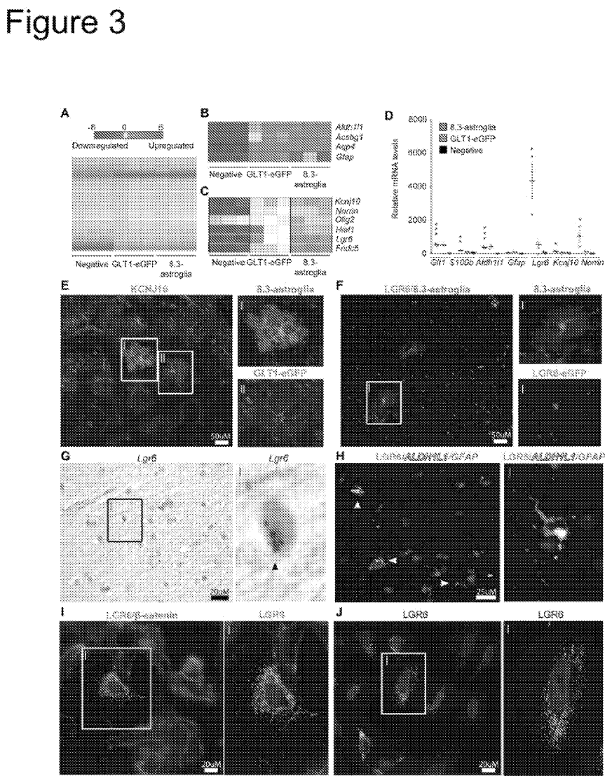

The inventors next assessed the unique molecular properties of the 8.3-astroglia by determining their transcriptomic profile. Adult mouse cortices (n=3) were dissociated into single-cell suspensions and separated by FACS to collect three distinct populations: GLT1-eGFP only (grey-matter astroglia), 8.3-astroglia (grey-matter astroglia subset), and the negative-fluorescent cell population (all cells that are not grey-matter astroglia) (FIG. 10A). During our FACS the inventors noted all astroglia displayed some level of tdTomato-fluorescence, however they were able to isolate only the high expressers which are continued to be referred to as 8.3-astroglia (FIG. 10A). Based on microarray analysis of these populations, each group displayed a unique RNA transcriptome (FIG. 3A). As expected, the two astroglia populations were both enriched in well-established astroglia markers such as Aldh111, Acsbg1, Aqp4, and Gfap (FIG. 3B). However, when comparing 8.3-astroglia to GLT1-eGFP astroglia and the reporter-negative cell population, the 8.3-astroglia in the cortex displayed consistent and profound enrichment in several candidate markers (FIG. 3C-D, FIG. 25). Some of the highest expressing genes, that have also been shown to be involved in neurological disorders, include Kcnj10, Norrin, Olig2, Lgr6, and Fndc5 (FIG. 3C-D, FIG. 25); their expression was validated by quantitative PCR (FIG. 3D).

Hypothesizing that these enriched proteins could provide clues into the unique biological properties of these adult astroglia subpopulations, the inventors generated a list of markers enriched in single-positive GLT1-eGFP astroglia and double-positive astroglia populations and subjected these modified lists to software-based pathway analytics. The inventors found that these astroglia populations were uniquely enriched in specific pathways, with enrichment of the Wnt/β-catenin signaling pathway in GLT1-eGFP astroglia and enrichment of the Sonic Hedgehog pathway in 8.3-astroglia (FIG. 10D). Both astroglia populations showed enrichment in pathways common to the function of all astroglia, such as pathways involved in cholesterol and fat synthesis. (FIG. 10D).

LGR6 and Norrin Expression by 8.3-Astroglia

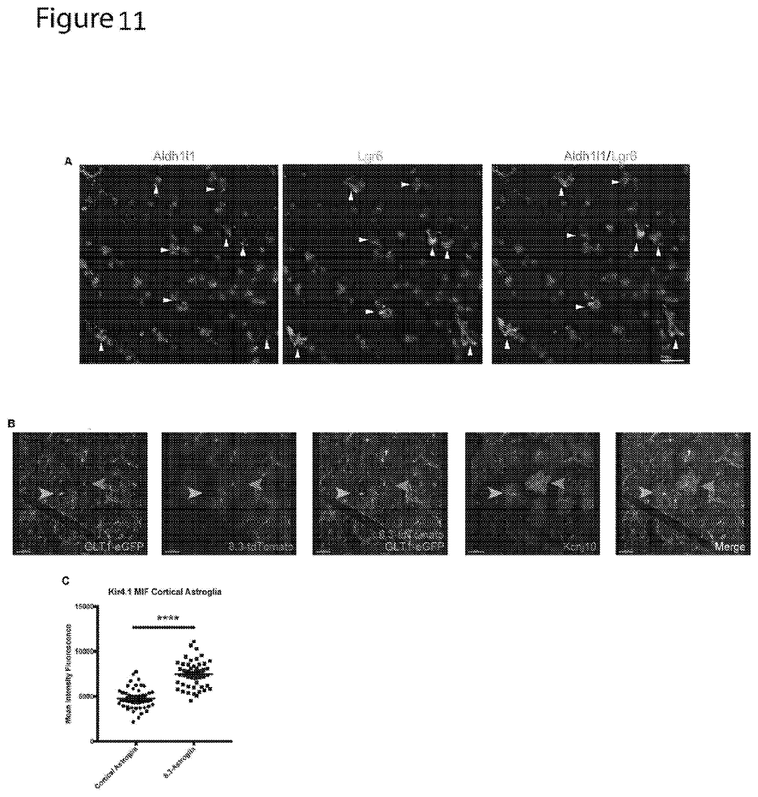

The inventors next sought to generate a panel of markers that could be used to uniquely identify 8.3-astroglia. Starting with candidates identified by our transcriptomic analyses, the inventors assessed four genes that were highly enriched in 8.3-astroglia compared to both the GLT1-eGFP and negative-fluorescent cell populations: Kcnj10, Lgr6, Olig2, and Norrin (FIG. 3C-D). In agreement with our RNA analyses, the cortical distribution of KCNJ10 was enriched in similar regions as the 8.3-astroglia (FIG. 3E, FIG. 11B-C). At higher magnification, 8.3-astroglia displayed a higher mean fluorescence intensity of KCNJ10 compared to GLT1-eGFP astroglia (FIG. 3E, FIG. 11B—C). To visualize Lgr6-expressing cells, we generated a double transgenic mouse model by crossing LGR6-GFP-ires-CreERT2 mice with the 8.3-astroglia mice. As expected, LGR6-eGFP localized to all 8.3-astroglia in the adult cortex, with varying degrees of fluorescence, of these double transgenic mice (FIG. 3F). The LGR6-EGFP fluorescence was only noted in 8.3-astroglia and no other cells. The inventors also detected nuclear OLIG2 expression in 8.3-astroglia, consistent with the microarray data (FIG. 10E). The inventors note that although OLIG2 is widely used to identify oligodendrocyte lineage cells in the CNS, it is also known to exist in some neuroprotective astroglia subpopulations. In aggregate, the molecular identity and the unique CNS localization of the 8.3-astroglia are useful for elucidating the biological significance of this glial subgroup and allows for the studying of this subpopulation with the need of the transgenic mice.

To determine whether this astroglia subpopulation is also present in the human cortex, the inventors used LGR6 expression as a surrogate marker for the 8.3-astroglia population. Using immunohistochemistry and RNA in situ hybridization on post-mortem adult human cortex tissue, the inventors detected LGR6 expression in only a subset of astroglia in the human cortex, consistent with the results of our rodent studies (FIG. 3G-H, 11A). To validate the astroglia-specific expression of LGR6 in humans, the inventors used immunohistochemistry to visualize astrocyte-specific marker ALDH1L1. LGR6 colocalized only with ALDH1L1-positive cells in the adult human cortex, providing further support that these cells are indeed a subset of human cortical astroglia (FIG. 3H, FIG. 11A).

LGR6-positive astroglia subsets could also be reliably identified in vitro in both pure mouse primary cortical cultures and in human induced pluripotent stem (hiPSCs) differentiated into astroglia (FIG. 3I-J). The inventors also observed a higher colocalization of KCNJ10 and LGR6 in hiPSC-derived astroglia, supporting the results of our combinatorial profile (not shown). Thus, these studies establish a well-defined and geographically organized astroglia subpopulation in the adult human and rodent cortex and provide reliable markers to study their involvement in normal and diseased CNS physiology.

Functional Assessment of LGR6 in Astroglia

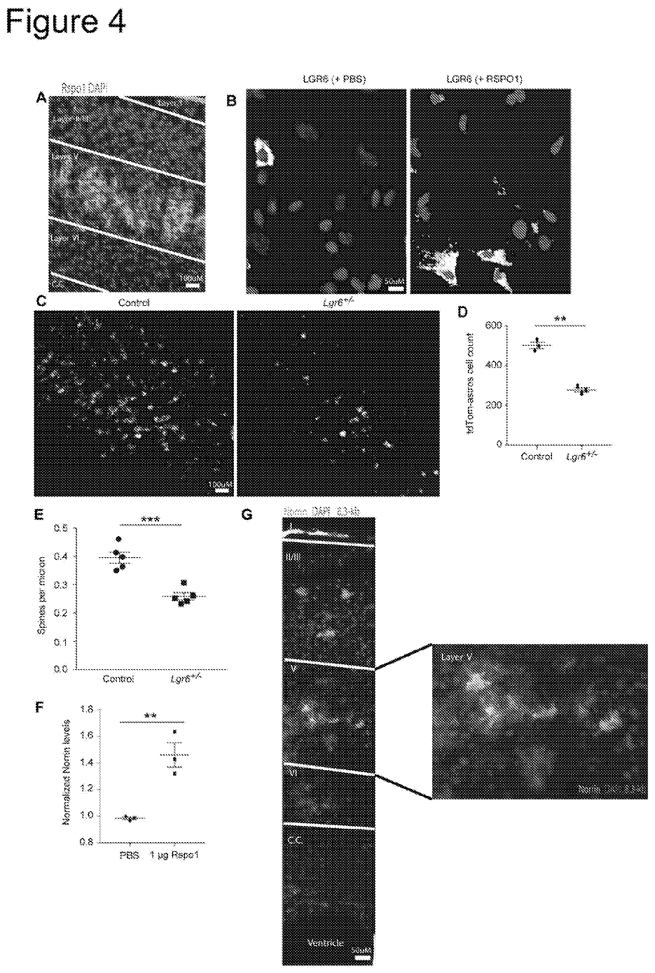

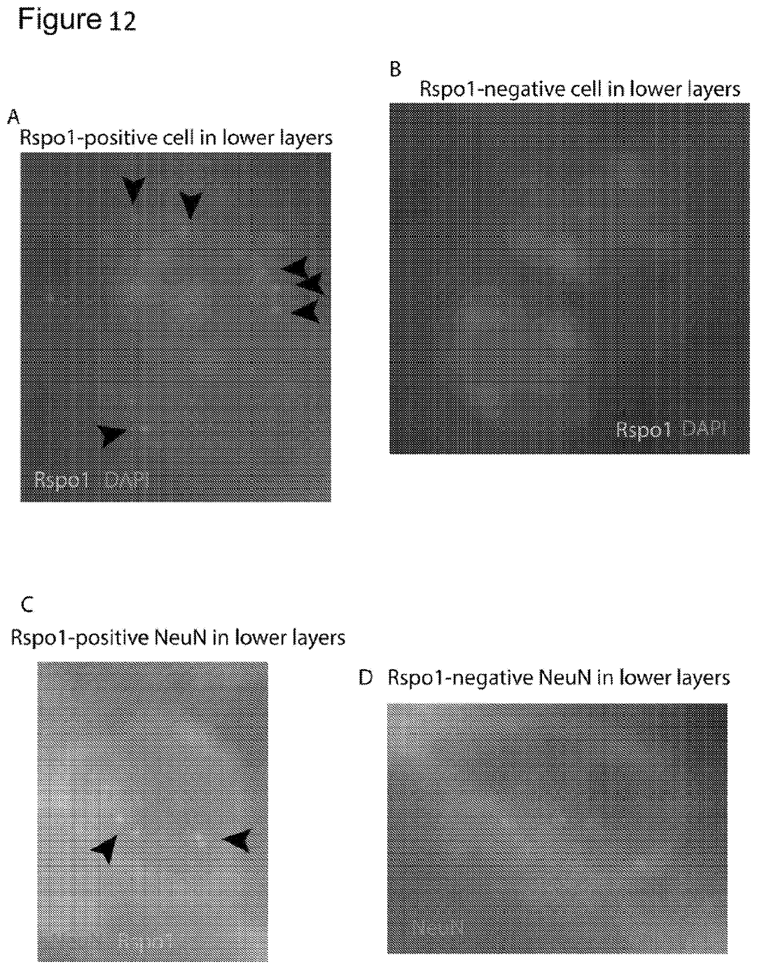

The inventors next aimed to determine the functional significance of our observation that the receptor LGR6 is consistently enriched and labels this astroglia subpopulation. In addition, the inventors focused on LGR6 because it showed the highest enrichment in our RNA analytics and has been widely shown to be astroglia-specific in the CNS. To address this question, the inventors investigated the effects of addition of its ligand, R-spondin (RSPO1). RSPO1 has been shown to be neuron-specific in the adult cortex, but little is known about the downstream consequences of its interaction with LGR6 in the CNS. Consistent with published RNA in situ hybridization data, RSPO1 was highly enriched in areas of dense 8.3-astroglia in layer V (FIG. 4A, FIG. 12A-D). Furthermore, the inventors performed immunofluorescence for neuronal marker, NeuN, followed by RNA fluorescent in situ hybridization (FISH) for Rspo1 and noted that Rspo1 co-localized only to NeuN-positive neurons but only in a subset in the lower cortical layers (FIG. 12A-D). This provides additional support that Rspo1 is neuronal-specific in the adult motor cortex but that it is also limited to only a subset of neurons in cortical layer V.

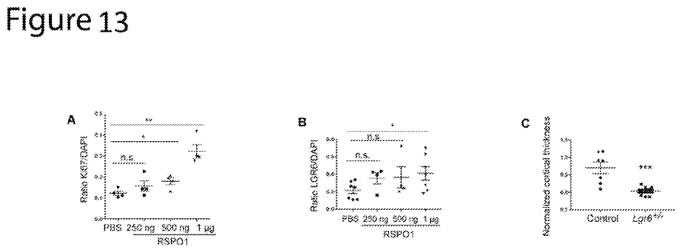

Next, the inventors wanted to explore the astroglia response to RSPO1 in vitro. Treatment of primary astroglia cultures with increasing doses of RSPO1 resulted in significant enrichment in the overall numbers of LGR6-positive astroglia compared to PBS-treated control cultures (FIG. 4B, FIG. 13A-B). To determine whether this increase in LGR6-positive astroglia was due to proliferation, the inventors stained astroglia with the proliferation marker Ki67, revealing a significant dose-response increase of Ki67-positive astroglia following RSPO1 treatment (FIG. 13A). This increase in proliferation was due to an increase of LGR6-positive astroglia (FIG. 13B). These in vitro findings suggest that in postnatal conditions, local neuronal release of RSPO1 could stimulate proliferation of astroglia, suggesting a specific interaction between a RSPO-positive neuron subpopulation and LGR6-positive 8.3-astroglia.

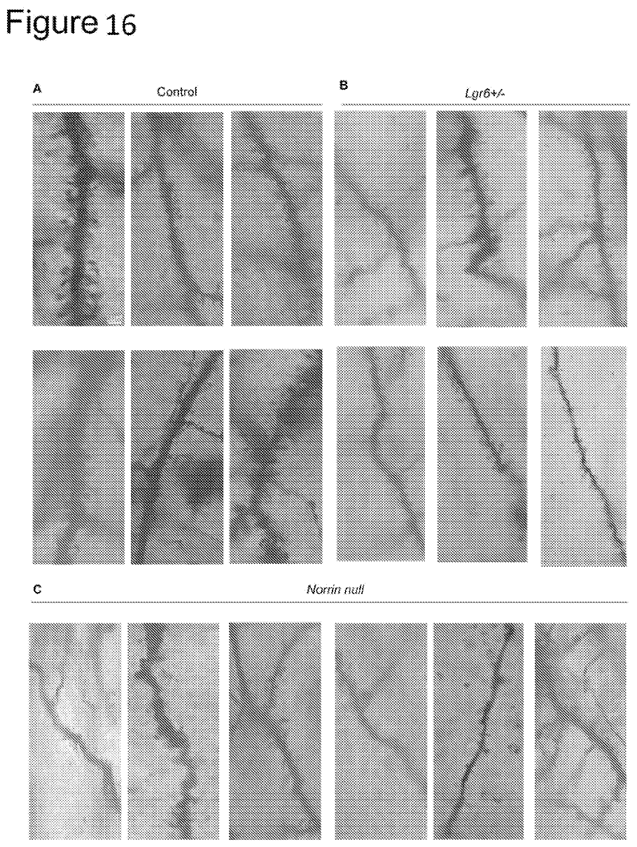

The stimulatory effect of LGR6 on proliferation of 8.3-astroglia in vitro suggested that loss of LGR6 could decrease the population of 8.3-astroglia. Indeed, the inventors observed a dramatic loss of 8.3-astroglia in vivo in the heterozygote 8.3-astroglia/LGR6-GFP-ires-CreERT2 mice, which have 50% lower expression of LGR6 compared to wild-type mice; this decrease further correlated with a minimal but significant loss of cortical thickness (FIG. 4C-D, FIG. 13C). Since astroglia play a major role in neuronal synapse formation and elimination, the inventors analyzed the overall density of spines on apical dendrites in layer V where 8.3-astroglia are highly enriched. The inventors found a significant decrease in spine density in the heterozygote LGR6-eGFP/8.3-astroglia mouse model, correlating with the loss of 8.3-astroglia (FIG. 4E, FIG. 16A-B).

Next, the inventors sought to determine which factor(s) released by 8.3-astroglia could be responsible for the deficits in dendritic spine density, focusing on secreted proteins that were highly enriched in 8.3-astroglia as identified by our transcriptomic analyses. To test for secretion of neuro-modulating proteins the inventors performed stimulation of LGR6-positive astroglia with RSPO1 and used ELISA to detect candidate secreted proteins and determine which proteins increased with RSPO1 treatment. One highly abundant protein was Norrin (Ndp, Norrie disease protein) (FIG. 4F), a protein that is expressed in vivo by cortical astrocytes with strong colocalization with 8.3-astroglia in cortical layer V and has been shown to be astroglia-specific in the cortex (FIG. 4G).

Norrin Release by 8.3-Astroglia Regulates Dendritic Growth and Spine Formation

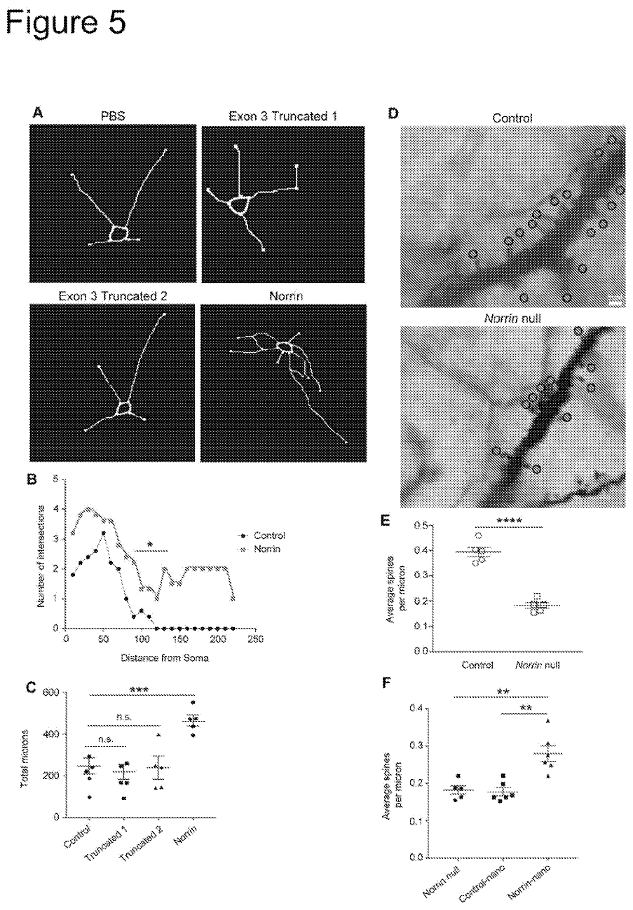

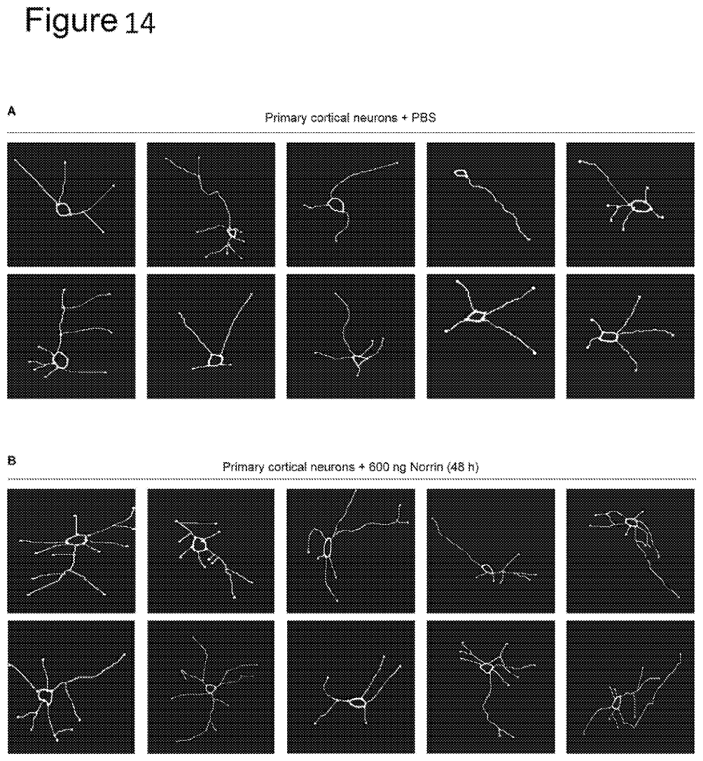

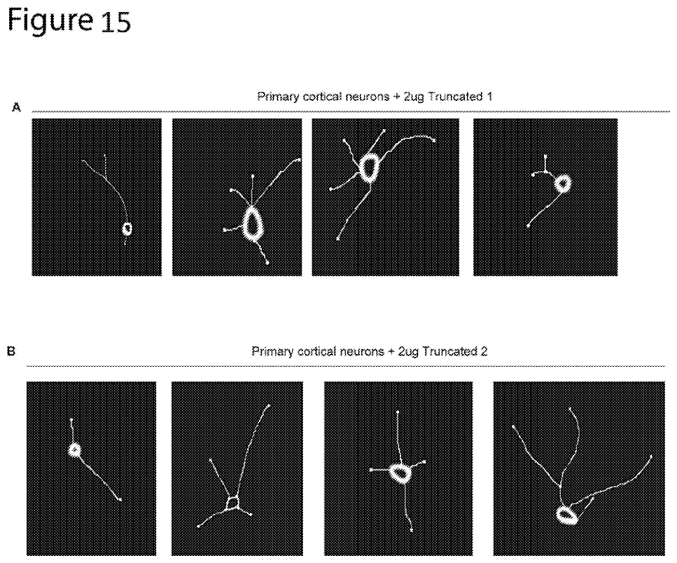

Norrin is known to be astroglia-specific in the adult cortex, where it binds to FZD4 and several other receptors such as LGR4 to activate the Wnt signaling pathway and thereby induce upregulation of neurotrophic growth factors, including Brain-Derived-Neurotrophic-Factor (BDNF). Patients with Norrin mutations develop Norrie disease, a CNS and ocular disease that also has strong cognitive and behavioral deficits including mental retardation, psychosis, and early-onset dementia. Some patients with Norrie disease have a deletion of exon 2, resulting in the possibility of two truncated proteins from exon 3. To evaluate the effects of increased Norrin on cortical neurons, the inventors treated primary mouse cortical neurons with recombinant Norrin, compared to the PBS vehicle control and the two Norrin truncated proteins from exon 3 (referred to as “Truncated 1” and “Truncated 2”) (FIG. 5A, FIG. 14A-B, FIG. 15A-B). The inventors found that treatment with both truncated proteins did not affect dendritic arborization or length, however Norrin significantly affected dendritic arborization and increased dendritic length (FIG. 5B-C; data not shown). These findings show that the truncated Norrin proteins translated in Norrie disease patients is inefficient at effecting neuronal dendrites.

Norrin has known effects on retinal biology, but its function in the cortex is unknown. To determine whether absence of Norrin would lead to neuronal deficits, we analyzed Norrin-null mice and quantified cortical neuron dendritic spine density. The inventors found a significant loss of spines in cortical layer V in Norrin-null mice compared to their control littermates (FIG. 5D-E, FIG. 16A, C).

Next, the inventors wanted to evaluate the effects of in vivo Norrin treatment on cortical neuronal dendritic spine density. To address this, the inventors created a genetic plasmid with the astroglia subset specific promoter 8.3 kb followed by Norrin cDNA and packaged this plasmid into nanoparticles that were injected into the mouse motor cortex. Norrin secretion driven solely by the 8.3 kb promoter significantly increased dendritic spine density in the Norrin-null mice compared to the Norrin-null and CMV-eGFP nanoparticle control (FIG. 5F). This provides evidence that Norrin production solely by 8.3-astroglia is sufficient to restore the dendritic abnormalities and suggested a role for this astroglial specific pathway in the regulation of neuronal spine and synaptic physiology.

Norrin Treatment Effects the Electrophysiological Properties of Neurons

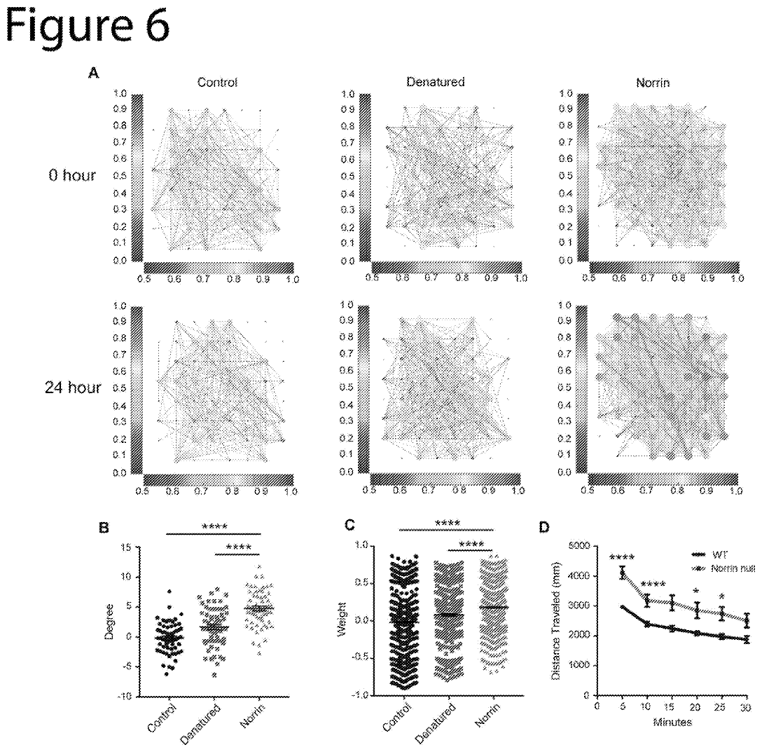

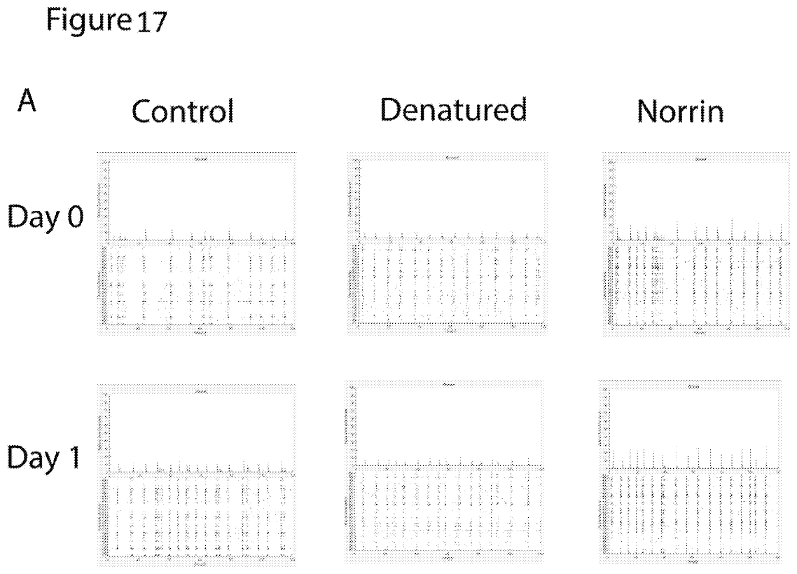

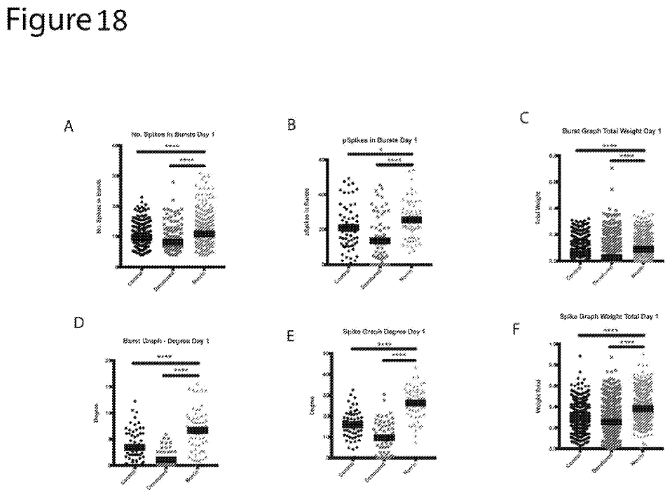

Next, the inventors wanted to explore the effects of Norrin on the electrophysiology of cultured neurons. Using multi-electrode array (MEA) plates with rat cortical neurons and astrocytes, the inventors monitored basal activity before and 24 hours after Norrin treatment (FIG. 6A, FIG. 17A, 18A-F, Videos 3-5). Spike and burst rates were normalized to pre-treatment activity to control for batch effects in baseline activity. There was a significant increase in the difference in both node degree and connection strength between electrodes, as well as a reorganization of spiking patterns wherein a greater percentage of detected spikes were organized into bursts (FIG. 6B-C, FIG. 18A-F). These findings demonstrate that Norrin could function to organize and strengthen cortical neuronal connectivity.

Norrin-Null Mice Display Neurobehavioral Abnormalities

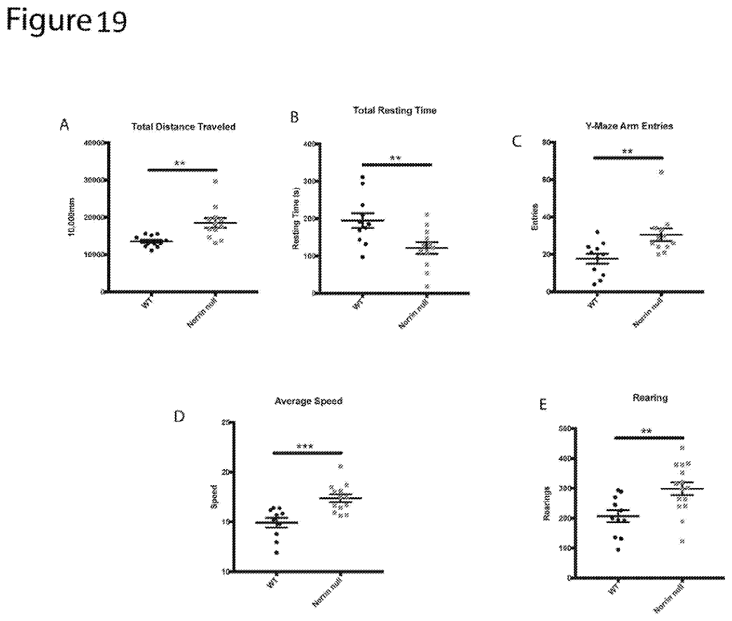

The Norrin-null mouse displayed a pronounced loss of dendritic spines in layer V of the cortex and effected both electrophysiology and dendrites in vitro, so the inventors sought to determine if they also displayed altered behavior. To test this hypothesis, they subjected the Norrin-null mice and their wildtype littermates to an array of behavioral assays. Surprisingly, in the open field assay, Norrin-null mice were significantly more hyperactive than their wildtype littermates, as shown by increased exploration and decreased resting time (FIG. 6D, FIG. 19A-B). In the Y-maze assay, Norrin-null mice displayed increased combinations and arm entries, supportive of a hyperactive phenotype (FIG. 19C). Overall, the average speed of Norrin-null mice was significantly higher than the controls (FIG. 19D). When evaluating rearing, Norrin-null mice had significant more rearing behaviors than their wildtype littermates (FIG. 19E). Lastly, in the catwalk assay, several parameters were also significantly altered in the Norrin-null mice (data not shown). Furthermore, at this time period (P60-P90), these mice did not display abnormalities in several assays such as elevated platform maze, fear conditioning, and rotarod (data not shown). These findings further support that Norrin-null mice have neurobehavioral abnormalities such as hyperactivity which may be the result of their cortical phenotype.

The majority of evidence for astroglia heterogeneity has arisen from developmental studies in the spinal cord or found as a pathological consequence in neurological disorder. To date, there is little data that molecularly and/or physiologically defines the presence of astroglia subsets in the adult nervous system that function to interact and/or regulate regional neuronal populations. Furthermore, no reliable tools exist to accurately and robustly identify these subpopulations in normal and disease states. With the generation of the 8.3-astroglia mouse line, the inventers have discovered and are now able to easily study a specific astroglia subset in the cortex of adult mice. This astroglia subset, 8.3-astroglia, has distinct cortical patterning and molecular profiles compared to other grey-matter astroglia, and can be robustly identified using unique markers such as OLIG2, NORRIN, KCNJ10 and LGR6 in rodent and human CNS, as well as in primary mouse and hiPSC-astroglia cultures.

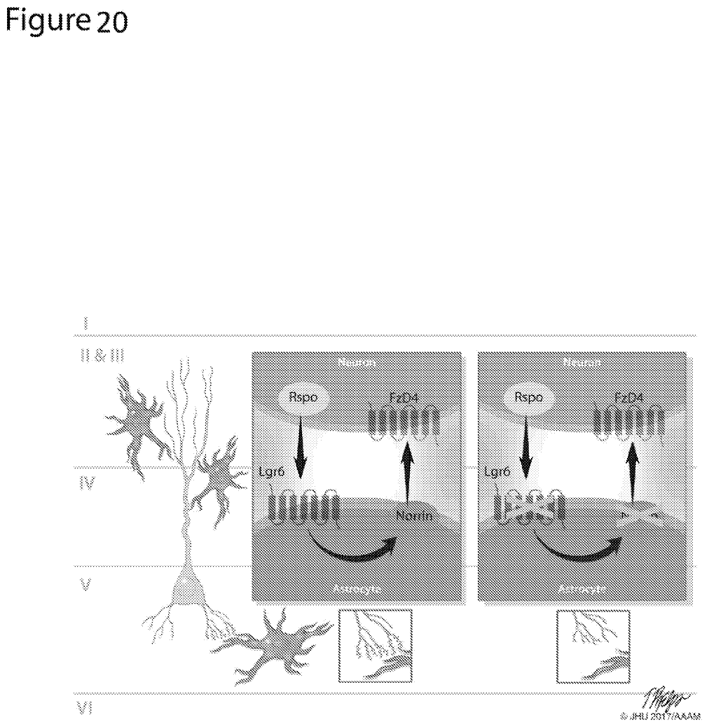

The inventors' extensive profiling data has allowed them to generate a protein/RNA profile to molecularly identify 8.3-astroglia. LGR6 colocalizes with 8.3-astroglia in the mouse and human cortex, along with subsets of in vitro astroglia from rodents and in hiPSC-derived astroglia. LGR6 contributes to Wnt signaling, one of the pathways enriched in grey-matter astroglia, and its ligand, RSPO1, is specifically released by pyramidal neurons. In the cortex of adult mice, the inventors demonstrate that RSPO1 is restricted to a neuronal subset in layer V, the areas highly enriched in LGR6-positive 8.3-astroglia. LGR6 and Norrin have both been shown to be highly enriched and limited to astroglia. The correlation of RSPO1, LGR6, Norrin, and 8.3-astroglia cortical patterning strongly suggests that there is crosstalk between neurons and astroglia, where extrinsic signaling from neuronal subtypes could influence astroglia subtype-specific responses (FIG. 20). In support of this model, the inventors find that release of RSPO1 from neighboring neurons activates the LGR6 pathway in 8.3-astroglia to induce proliferation and the release of Norrin in vitro. It is well known that neurons influence astroglial physiology, for example via astroglia expression of glutamate transporters EAAT2.

The unique anatomical localization of 8.3-astroglia and their enrichment in LGR6 and Norrin suggest that they may be involved in glial-based pathogenesis of Norrie disease. The inventors document a selective loss of spine density in both the LGR6 heterozygote and Norrin-null mice in cortical layer V, corresponding to the location of 8.3-astroglia and Norrin expression. This is particularly interesting as Norrie disease patients with genetic deletion of exon 2 of the Norrin gene developed epilepsy. Epileptic patients have been document to have reduced spine density and altered dendritic arbor. Unfortunately, it is not possible to analyze spine density on Norrie disease patients as there is no available post-mortem brain tissue.

Norrin's effect on cortical neuron dendrites is particularly interesting. The inventors show that treatment with Norrin on neurons leads to increases in dendritic length and arborization. However, treatment with the two truncated Norrin proteins found in Norrie disease patients with deletion of exon 2 exhibited no effect on neuronal dendrites. There is a loss of spines in a widespread of disorders. In an ALS mouse model, there is a significant loss of spines in pyramidal layer V neurons in the motor cortex. This is similar to the pathology in Alzheimer's disease, where dendritic spine loss is also involved. In transcriptomic data of Alzheimer's disease (AD) mice, Norrin is significantly downregulated in astrocytes. There is also an observed loss of Norrin levels in the hippocampus in AD. Finally, mental retardation (another common phenotype in Norrie disease patients) reduced spine density is also observed.

Norrin could serve as a potential therapeutic in neurological disorders with a spine density pathology. The inventors show that by using the 8.3 kb specific promoter to drive the expression of Norrin in the mouse cortex is sufficient at restoring and improving cortical spine density. This data shows that the sole release of Norrin strictly by 8.3-astroglia could serve as a therapeutic. Additionally, the usage of a ubiquitous promoter would hypothetically elevate Norrin levels even more and could lead to a more dramatic increase in spine density, alterations to dendritic arborization and length, and the electrophysiology of cortical neurons. In fact, the multielectrode array studies allowed us to test the effects of Norrin on the electrophysiological properties of neurons and Norrin treatment led to improved neuronal connectivity and strength supporting a role for this astrocyte subpopulation in regulating neuronal connectivity and a candidate therapy.

To assess the clinical phenotype of these mutant mice, the inventors performed a wide array of behavioral assays on the Norrin-null mice. The inventors found that Norrin-null mice displayed abnormal behavior in the open field assay, Y-maze, and catwalk assay. Overall the Norrin-null mice were hyperactive, which is similar to what has been shown in BDNF mutant mice and it has been shown that Norrin can drive the expression of BDNF. These neurobehavioral abnormalities are supportive of a cortical pathology in the Norrin-null mice.

Lastly, the focal localization of the cortical layer of 8.3-astroglia is quite intriguing. It has been shown recently that in the cortex, different layers exhibit different populations of astroglia, when based on their morphology. The inventors now show that the 8.3-astroglia subset are the dominant population of layer V astroglia. It is well established that different cortical layers exhibit different neuronal populations. This provides further evidence for a layer-specific cross-talk between subsets of neurons and subsets of astroglia leading to a high degree of diversity and functional specification. Future studies could explore this relationship, especially in disorders where a subset of cells is affected (i.e. ALS motor neurons).

Taken together, these findings begin to shed light on the complexity of astroglia heterogeneity by uncovering and defining a unique subset of astroglia in the CNS and has led to new tools to manipulate this astroglia subset in efforts to ultimately discover novel therapeutic avenues for neurological disorders affected by this and other astroglia subsets. This has also led to advances in understanding Norrie disease and the contributions that astroglia may play in the pathology. These studies set a foundation to build upon for understanding astroglial subset and unique neuronal glial pairing in normal and pathological states.

Next the inventors investigated a certain astroglia subpopulation, 8.3-astroglia, in a transgenic rodent model of ALS as well as in human autopsy tissue and ALS iPS cell lines. In these experiments, the inventors showed that this specific astroglial subpopulation was lost in areas of motor neuron degeneration, as are corresponding gene expression markers involved in the Wnt-signaling pathway and potassium ion buffering.

First, the inventors explored 8.3-astroglia in the ALS SOD1 G93A animal model. In the double transgenic 8.3-astroglia/SOD1 G93A end-stage mice, the inventors note a complete ablation of 8.3-astroglia in the lumbar spinal cord and partial loss in the murine motor cortex, consistent with surrogate markers of 8.3-astroglia (i.e. Kir4.1,Lgr6). Next, the inventors examined human post-mortem tissue from ALS patients and observed a significant loss of Lgr6-positive astroglia in the motor cortex, which was supported by their biochemical experiments. Lastly, the inventors showed in vitro that ALS patient human induced-pluripotent stem cells (hiPSC) differentiated into astroglia exhibit a dramatic loss of Lgr6-positive astroglia and Kir4.1-immunoreactivity. Taken together, these results showed for the first time that a specific astroglia subpopulation, and not all astroglia, were significantly affected in ALS pathophysiology.

8.3-Astroglia are Lost in the Motor Cortex and Lumbar Spinal Cord of End-Stage SOD1 G93A Mice

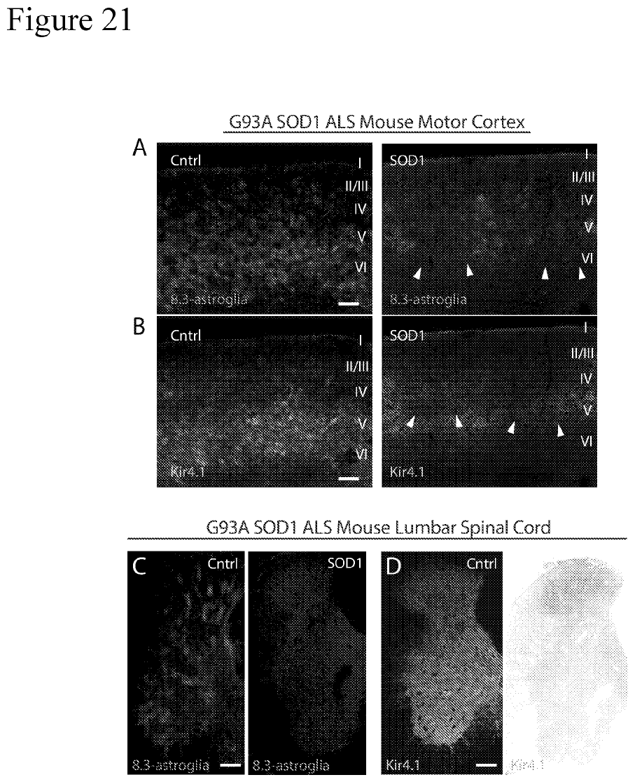

8.3-astroglia are an abundant astroglia subpopulation in the cerebral cortex and spinal cord of 8.3-kb transgenic mice. This astroglia subpopulation modulates cortical excitatory neuron dendritic spine density via secretion of a neuro-modulating protein, Norrin. In addition, this subgroup is enriched for expression of Lgr6, a member of the Wnt-signaling pathway, and also Kir4.1, a key potassium channel involved in several neurological disorders. Therefore, the inventors decided to evaluate this astroglia subgroup in the SOD1 G93A mice. Surprisingly, at end-stage the inventors noticed an extreme loss of 8.3-astroglia in the motor cortex that was accompanied by a similar loss in Kir4.1 (FIG. 21A-B). In the lumbar spinal cord, where motor neurons degenerate, the inventors found a complete ablation of 8.3-astroglia that was characterized by a change in the distribution of Kir4.1-immunoreactivity from the ventral horn to the dorsal horn (FIG. 21C-D).

Lgr6-Positive Astroglia are Lost in the c9orf72 Motor Cortex

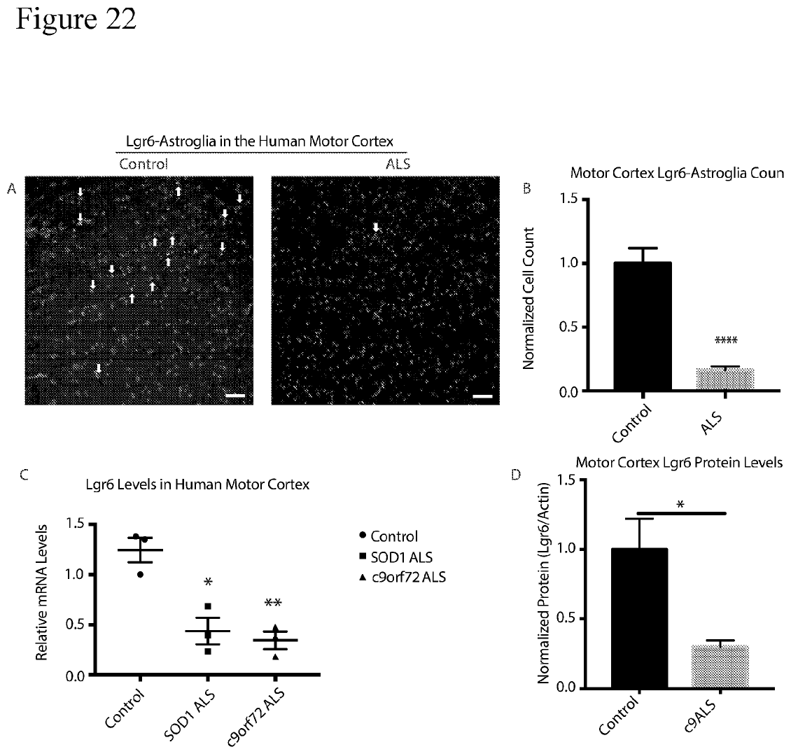

Given the loss of 8.3-astroglia in both motor cortex and lumbar spinal cord of end-stage SOD1 G93A mice, the inventors next evaluated human post-mortem tissue from ALS patients. 8.3-astroglia were recently shown to be present in human post-mortem brain tissue (manuscript in revision). Recent microarray data obtained from c9orf72 patient motor cortex shows a significant downregulation in 8.3-astroglia surrogate markers such as Lgr6 and its cognate, neuron-specific ligand R-spondin 1. Similar to our unpublished transcriptomic data, the inventors saw a dramatic loss of Lgr6-positive astroglia in the c9orf72 ALS (c9ALS) motor cortex compared to control (FIG. 22A-B). Next, the inventors examined the relative Lgr6 mRNA and protein levels. In support of our histology, the inventors noted a significant and robust loss of Lgr6 mRNA and protein levels (FIG. 22C-D).

Lgr6-Positive hiPSC-Astroglia are Lost in c9orf72 and SOD1 ALS Patient Lines

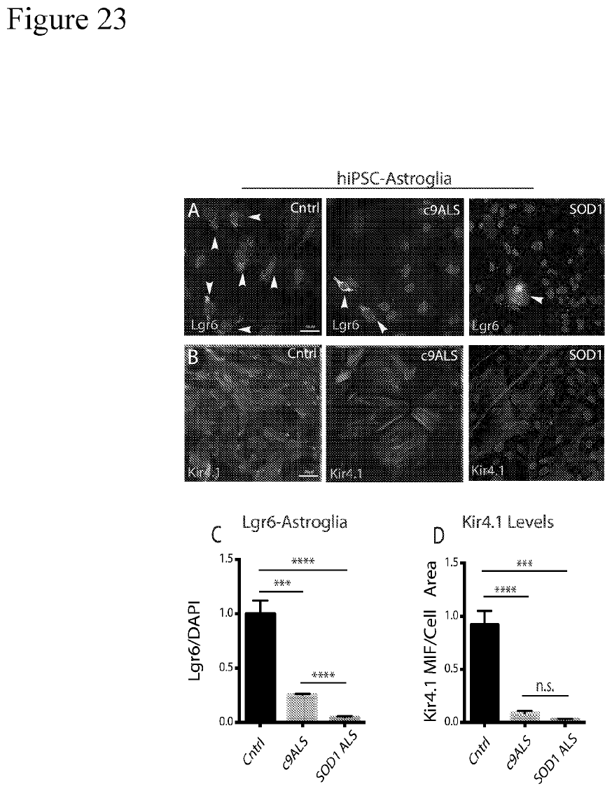

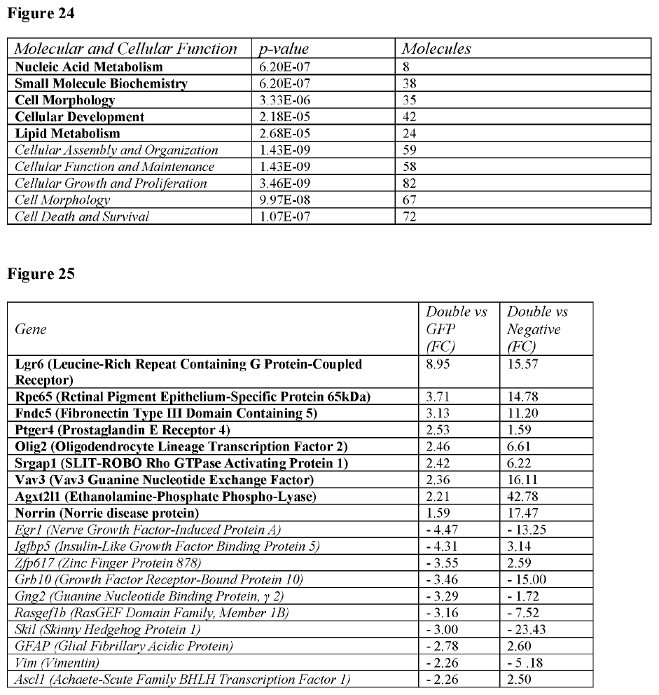

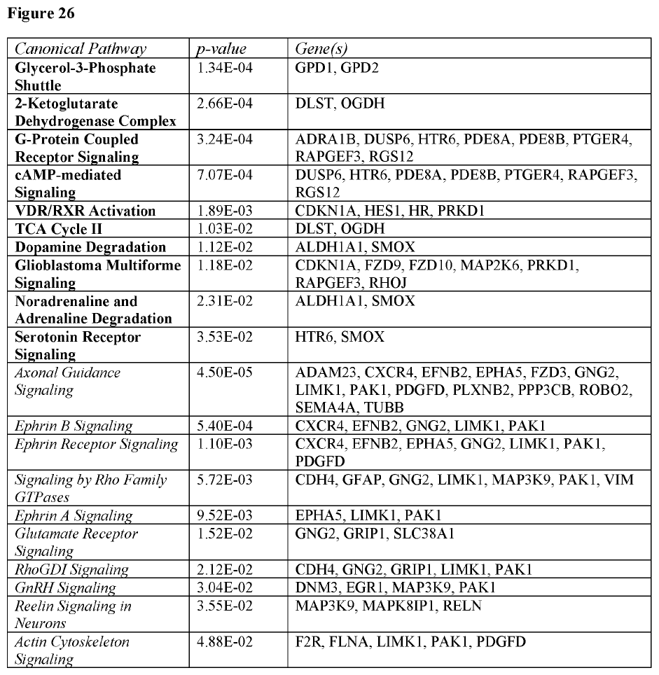

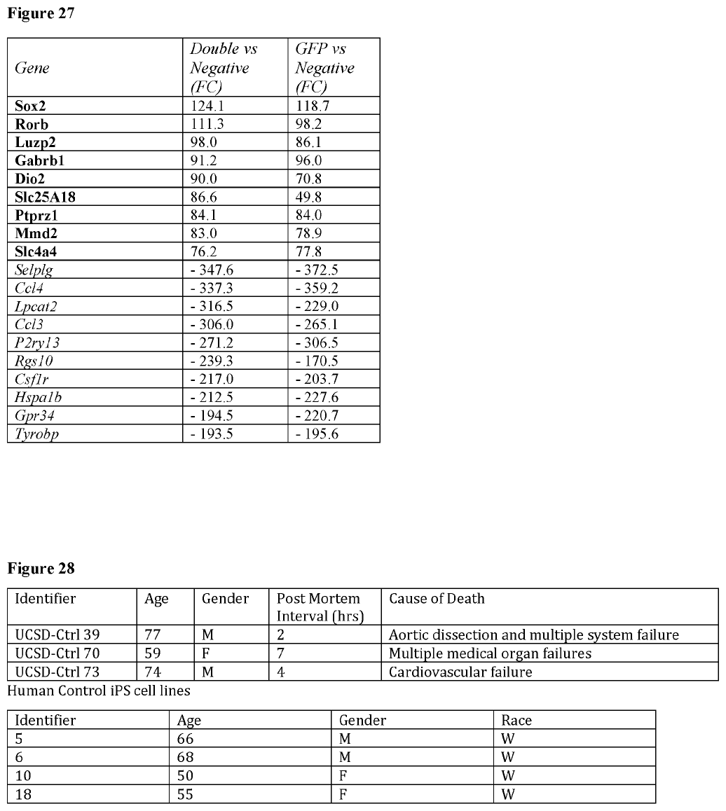

hiPSC-astroglia derived from ALS patients are toxic to motor neurons in culture. To examine Lgr6-positive astroglia in hiPSC-astroglia, the inventors first performed immunofluorescence on mature astroglia. The inventors found that Lgr6- and Kir4.1-immunoreactivity was significantly lower in both c9orf72 (c9ALS) and SOD1 ALS hiPSC-astroglia as compared to controls (FIG. 23A-D). This supports the notion that ALS patients have either a dramatic loss of Lgr6-positive astroglia, or that Lgr6 and Kir4.1 genes and/or protein are significantly downregulated in ALS diseased astroglia.

To date, very little is known about astroglia heterogeneity. The majority of studies have been focused on development subtypes altered in pathological conditions. In ALS, not all astroglia, but instead a subset in the cortex and spinal cord, downregulate essential neurotransmitter transporters and ion channels. It is not surprising, that this astroglia-based dysfunction is localized to areas of neuronal degeneration. This focal dysregulation has been the focus of numerous studies, mainly regarding ALS as a non-cell autonomous disease, involving both neuroglia and neurons.

Both in vivo and in vitro studies show that diseased astroglia are neurotoxic to both healthy and diseased neurons. However, little is known as to whether or not this toxicity is due to a gain-of-function neurotoxicity, a loss-of-function related to neurotrophic signaling, and/or a combination of the two. Furthermore, no studies have concretely explored a single astroglia subtype, but rather they have focused on all astroglia as a homogeneous population. Therefore, the inventor's work is one of the very first to explore a unique subset of astroglia, the 8.3-astroglia in this degenerative disease.

8.3-astroglia are localized to areas of motor neuron death in ALS. In addition, 8.3-astroglia release proteins that enhance neuronal dendritic spine and dendritic branching, including Norrin, and this population of astroglia is enriched in specific ion channels such as Kir4.1. In the cortex of SOD1 G93A mice it has been shown that the mice experience neuronal spine regression and hyperexcitable neurons. The inventors show that 8.3-astroglia are significantly lost in this area of motor cortex. This suggest that some of the cortical phenotypes seen in the ALS model may be due to a loss of 8.3-astroglia and/or their enriched markers.

Next, using markers that are selectively enriched in 8.3-astroglia, the inventors explored human post-mortem brain tissue. The inventors focused on the marker Lgr6, as this has been shown to specifically and robustly label 8.3-astroglia in vivo and in vitro (manuscript in revision). After performing an extensive immunofluorescence analyses on ALS and control patient motor cortex, the inventors found that Lgr6-positive astroglia were dramatically reduced in number. These data, in combination with our ALS mouse data, support the notion that 8.3-astroglia/Lgr6-positive are significantly lost in both the human and mouse motor cortex. This is supported by biochemical approaches evaluating mRNA and protein levels.

Lastly, the inventors utilized the in vitro model of human induced-pluripotent stem cells differentiated into astroglia. When detecting levels of Lgr6-positive and Kir4.1-enriched astroglia, the inventors found a significant alteration in diseased specimens. First, the inventors noted a dramatic loss of Lgr6-positive in both the c9orf72 and SOD1 human patient IPS lines compared to control. Secondly, the inventors found that this loss in Lgr6 was accompanied by significant downregulation of Kir4.1 levels in these same human astroglia cell lines. Taken together, this study demonstrates that perhaps not all, but rather a subset of astroglia are affected in ALS, and that targeting this subtype may lead to potential therapeutic approaches. Unraveling the exact influence, either neurotoxic or neurotrophic, of this astroglia subset on neurons should be the scope of future studies.

Embodiments of the disclosure concern methods and/or compositions for treating and/or preventing a neurological disorder including dendritic spine loss such as amyotrophic lateral sclerosis (ALS), for example, in which modulation of the Norrin pathway is directly or indirectly related. In certain embodiments, individuals with a neurological disorder such as ALS are treated with a modulator of the pathway. In specific embodiments an individual with ALS is provided a modulator of the activity of LGR6, such as an agonist of LGR6 including RSPO1, that induces the expression of Norrin, for example. In certain embodiments, individuals, with a neurological disorder including dendritic spine loss including ALS, are provided with a pharmaceutical composition comprising a Norrin protein, or functional part thereof, to treat or prevent the disorder. Alternative vectors expressing modulators of the Norrin pathway, such as the Norrin protein or RSPO1 may be administered to a subject to treat a condition of dendritic spine loss.

In certain embodiments, the level to which an inducer of Norrin expression increases Norrin expression may be any level so long as it provides amelioration of at least one symptom of the neurological disorder, including ALS. The level of expression may increase by at least 2, 3, 4, 5, 10, 25, 50, 100, 1000, or more fold expression compared to the level of expression in a standard, in at least some cases. An individual may monitor expression levels of Norrin using standard methods in the art, such as northern assays or quantitative PCR, for example.

An individual known to have a condition of dendritic spine loss, suspected of having a condition of dendritic spine loss, or at risk for having a condition of dendritic spine loss may be provided an effective amount of an inducer of Norrin expression, including RSPO1 an agonist of LGR1. Those at risk for a condition of dendritic spine loss may be those individuals having one or more genetic factors, may be of advancing age, and/or may have a family history, for example.

In particular embodiments of the disclosure, an individual is given an agent for a condition of dendritic spine loss therapy in addition to the one or more inducers or modulators of Norrin. Such additional therapy may include administering Norrin protein, or functional parts thereof to a subject, for example. When combination therapy is employed with one or more inducers or modulators of Norrin, the additional therapy may be given prior to, at the same time as, and/or subsequent to the inducer or modulator of Norrin.

Pharmaceutical Preparations

Pharmaceutical compositions of the present invention comprise an effective amount of one or more inducers or modulators of the expression of Norrin, such as RSPO1, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that comprises at least one inducer of expression of Norrin or additional active ingredient will be known to those of skill in the art in light of the present disclosure, as exemplified by Remington: The Science and Practice of Pharmacy, 21^{st} Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The inducer of expression of Norrin, or Norrin, may comprise different types of carriers depending on whether it is to be administered in solid, liquid or aerosol form, and whether it need to be sterile for such routes of administration as injection. The present compositions can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The inducer of expression of Norrin, or Norrin, may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include inducer of expression of Norrin, Norrin, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the inducer of expression of Norrin, or Norrin, may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

Alimentary Compositions and Formulations

In one embodiment of the present disclosure, the inducers of expression of Norrin, or Norrin, are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Parenteral Compositions and Formulations

In further embodiments, inducer of expression of Norrin, or Norrin, may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by FDA Office of Biologics standards.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

Miscellaneous Pharmaceutical Compositions and Formulations

In other preferred embodiments of the invention, the active compound inducer of expression of Norrin, or Norrin, may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998), nanoparticles described herein, and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

Kits of the Disclosure

Any of the compositions described herein may be comprised in a kit. In a non-limiting example, an inducer of expression of Norrin (for example, RSPO1), or Norrin, may be comprised in a kit.

The kits may comprise a suitably aliquoted inducer of expression of Norrin, or Norrin, and, in some cases, one or more additional agents. The component(s) of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the inducer of expression of Norrin, or Norrin, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred. The inducer of expression of Norrin, or Norrin, composition(s) may be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

### METHODS/EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The following Examples are offered by way of illustration and not by way of limitation.

Animal Models

Wild type (C57/BL6), LGR6-GFP-ires-CreERT2 (Jackson Laboratory), Norrin-null (gift from Jeremy Nathans), BAC-GLT1-eGFP, 2.5 kb-tdTomato, 6.7 kb-tdTomato, 7.9 kb-tdTomato, and 8.3 kb-tdTomato mice were used for in vivo experiments. GLT1/EAAT2-tdTomato transgenic mouse were generated by inserting the tdTomato reporter downstream of a 2.5 kb, 6.7 kb, 7.9 kb or 8.3 kb EAAT2/GLT1 promoter fragment as detailed in part previously. Multiple founders were established following pronuclear injection at the transgenic core laboratory of Johns Hopkins University.

The care and treatment of animals were in accordance with the NIH Guide for the Care and Use of Laboratory Animals, the Guidelines for the Use of Animals in Neuroscience Research, and the Johns Hopkins University IACUC. Mice were housed at standard temperature (21° C.) in a light-controlled environment with ad libitum access to food and water. BAC-GLT1-eGFP mice were crossed with 8.3 kb-tdTomato mice to generate double transgenic mice. 8.3 kb-tdTomato mice were also bred with LGR6-GFP-ires-CreERT2 mice. Littermates were used as controls and for comparisons between different astroglia populations.

Generation of GLT1/EAAT2 Promoter tdTomato Transgenic Mice

GLT1/EAAT2 (2.5 kb)-tdTomato: Fluorescence protein tdTomato was cloned into downstream of the human EAAT2 promoter sequence (2.5 kb). This DNA fragment was performed pronuclear injection to produce the transgenic mouse. Promoter sequence was based on the following: chr11:35440759-35443219, 2461 bp.

GLT1/EAAT2 (6.7 kb)-tdTomato Fluorescence protein tdTomato was cloned into downstream of the human EAAT2 promoter sequence (6.7 kb). This DNA fragment was performed pronuclear injection to produce the transgenic mouse. Promoter sequence was based on the following: chr11:35439159-35449110, 6752 bp.

GLT1/EAAT2 (7.9 kb)-tdTomato Fluorescence protein tdTomato was cloned into downstream of the human EAAT2 promoter sequence (7.9 kb). This DNA fragment was performed pronuclear injection to produce the transgenic mouse. Promoter sequence was based on the following: chr11:35439159-35448435, 7677 bp.

GLT1/EAAT2 (8.3 kb)-tdTomato: Fluorescence protein tdTomato was cloned into downstream of the human EAAT2 promoter sequence (8.3 kb). This DNA fragment was performed pronuclear injection to produce the transgenic mouse. Promoter sequence was based on the following: chr11:35440759-35449110, 8352 bp Nanoparticle preparation and injection

Brain-penetrating DNA nanoparticles (DNA-BPN) were formulated as previously reported⁴¹. Briefly, PEI was dissolved in ultrapure water and pH was adjusted to 7.5. Methoxy-PEG-N-hydroxysuccinimide (mPEG-NHS, 5 kDa; Sigma-Aldrich) was conjugated to 25 kDa branched PEI (Sigma-Aldrich) to yield highly PEGylated PEI (PEG-PEI). The synthesis of PEG-PEI was confirmed by nuclear magnetic resonance. GFP-encoding plasmid DNA driven by cytomegalovirus (CMV) promoter (pEGFP) was purchased from Clontech Laboratories. DNA-BPN were formulated by a dropwise addition of 10 volumes of DNA (pEGFP or 8.3 kb-tdTomato at 0.2 mg/ml) to 1 volume of swirling polymer solution. Polymer solution was prepared at nitrogen to phosphate (N/P) ratio, indicative of polymer to DNA ratio, of 6 and at PEG-PEI to PEI molar ratio of 3. After 30 minutes of incubation in room temperature, DNA-BPN were washed with three volumes of ultrapure water and concentrated to 1 mg/ml using Amicon Ultra Centrifugal Filters (100,000 MWCO; Millipore) to remove free polymers. DNA concentration in DNA-BPN was determined using a NanoDrop ND-1000 spectrophotometer (Thermo Fisher).

Generation of Norrin Truncated Proteins

Recombinant proteins were generated and obtained from GenScript. The following amino acid sequences were used:

Truncated protein 1:
(SEQ ID NO. 13)
MVLLARCEGHCSQASRSEPLVSFSTVLKQPFRSSCHCCRPQTSKLKALRL
RCSGGMRLTATYRYILSCHCEECNSGPLLCVASGWDNCRGSSTSQGKTGK
KRVKAKKDATILPGLCIF
Truncated protein 2:
(SEQ ID NO: 14)
MLRGHATHCHLPVHPLLSLRGMQFLRPAAVCGFWMGQL

In Vitro Adult 8.3-Astroglia Dissociation and Culture

Briefly, the Miltenyi Biotec Octo Dissociator was used with the adult CNS dissociation kit (MACS Miltenyi Biotec). Adult 8.3-astroglia/GLT1-eGFP mice (P60-P90, n=3) were perfused with PBS and the cortex was dissected. Astroglia were isolated according to the manufacturer's protocol. Dissociated cells were cultured on Matrigel-coated flasks. Cells were imaged every other day for up to 14 days, prior to passaging, on an inverted Olympus scope.

Human iPSC-Astroglia Differentiation

Patient fibroblasts were collected at Johns Hopkins Hospital with patient consent (IRB protocol: NA 00021979) as described. In vitro hiPSC-astroglia were cultured and differentiated as previously described. Briefly, iPSCs were converted into rosettes and then subjected to neural induction toward the neural progenitor cell (NPC) fate via SMAD inhibition. After differentiation into NPCs, treatment with fetal bovine serum was added to induce astrogenesis. Astroglia-differentiating NPCs were subjected to cell passaging once confluency reached 80-90%. All astroglia were analyzed at the mature state (12-13 weeks) and verified with appropriate astroglia markers as previously published. Patient demographics are available in FIG. 28. Cells were differentiated and imaged at age-matched time points.

Cell Treatments

Primary mouse astroglia cultures were treated with 250 ng to 1 μg of mouse recombinant carrier-free RSPO1 (R&D Biosystems) for 72 h. For cortical neuronal experiments, primary cortical neurons were isolated, matured for 7-10 days, and then treated with 600 ng-2 ug of mouse recombinant carrier-free Norrin (R&D Biosystems) or truncated protein 1 or truncated protein 2 or 1×PBS or DKK-1 for 24 h.

Fluorescence-Activated Cell Sorting (FACS)

Cortex, spinal cord, or cerebellum from BAC-GLT1-eGFP/8.3 kb-tdTomato double transgenic reporter mice (age P60-P90, n=3 male mice per experiment) were analyzed with FACS. Mice were anesthetized with an intraperitoneal injection of ketamine xylazine. Brain tissue was immediately dissected and dissociated as previously described. Single cells were then sorted using a MoFlo MLS high-speed cell sorter and gated based on eGFP and tdTomato fluorescence (FIG. 8D).

Microarray

Microarray procedure and analyses were performed as previously described. Briefly, total RNA was isolated from FACS-sorted populations using an RNA isolation kit (Qiagen) and the concentration was determined by NanoDrop (Thermo Fisher) and Bioanalyzer (Agilent Technologies). Only samples with a RIN score >5 were used. Total RNA was lineally amplified and labeled according to a Nugene protocol. Sample labeling and hybridization with Mouse Exon 1.0 ST chips (Affymetrix) were performed in the Johns Hopkins University Deep Sequencing and Microarray Core following the manufacturer's protocol. After hybridization, hybridization signals were acquired and normalized using Partek Genomics Suite software (Partek). Differential gene expression between conditions was assessed by statistical linear model analysis using Partek, Tibco Spotfire, and Prism 7 (GraphPad) software. The moderated t-statistic p-values derived from the Partek analysis above were further adjusted for multiple testing by Benjamini and Hochberg's method to control false discovery rate (FDR). An FDR cutoff of <10% was used to obtain the list of differentially expressed genes. Tissue from at least three mice were used for each microarray analysis. (Note: Data deposition will be submitted with final manuscript.)

Pathway Analyses

Gene ontology and pathway analyses were performed from data obtained from Partek and Spotfire post-statistical analyses using the Ingenuity Pathway Analysis software package as previously described.

Histological Analytics

Immunohistochemistry-Brain

Mice were injected with a lethal dose of ketamine xylazine and immediately perfused with 1×PBS followed by 4% paraformaldehyde. Post-perfusion, brain, cerebellum, and spinal cord tissue were collected and cyroprotected in 30% sucrose. Tissue samples were prepared for cryostat sectioning and sectioned at a thickness of 20 μm. Sections were treated with blocking buffer (5% donkey serum and 0.1% Triton-X 100 in PBS) for 60 min at room temperature. The following primary antibodies were used: GFP (Rockland), OLIG2 (Millipore), GFAP (Millipore), LGR6 (Abcam, R&D Systems), KCNJ10 (Alomone Labs), RSPO1 (Abcam), Norrin (Abcam), and ALDH1L1 (Abcam). Secondary antibodies were donkey anti-rabbit Alexa Fluor 647 (Abcam), donkey anti-mouse Alexa Fluor 647 (Abcam), donkey anti-rabbit 488 (Abcam), donkey anti-mouse 488 (Abcam), donkey anti-rabbit Cy3 (Abcam), and donkey anti-mouse Cy3 (Abcam). Labeled cells were manually counted or overall fluorescence calculated in Zen Zeiss 2012 software or using spot detection in Bitplane Imaris software 8.3.1⁴⁵. All images were captured using a Zeiss LSM 700 confocal microscope, Zeiss Axioimager, or Zeiss LSM 800 confocal microscope. A minimum of three mice per group were used for each experiment at ages consistent with the microarray experiments (P60-P90).

Immunocytochemistry—Cell Culture

Cells were stained as previously described. Briefly, cells were grown until the appropriate time point or confluency followed by fixation in 4% PFA and 0.3% Triton-X-100 permeabilization. Cells were then stained as described above (see Immunohistochemistry).

Golgi-Cox Staining

Tissue was processed according to manufacturer guidelines (FD Rapid GolgiStain Kit, FD Neurotechnologies).

CLARITY-Optimized Light-Sheet Microscopy (COLM)

Samples in movie 1 (n=2) were imaged using CLARITY-optimized light-sheet microscopy by mounting in a quartz cuvette. The objectives were immersed in a custom immersion liquid with RI of 1.454 (Cargille Labs). For illumination, 4×/0.28 NA/29.5 mmWD (Olympus) objectives were used and a 10×/0.6 NA/8 mmWD (Olympus) objective was used for detection. The image volume was acquired with 5 μm z-step size. The volume rendering was performed using 4×4 fold down-sampled data with Amira (FEI).

Tissue Clearing

A passive CLARITY method was used for tissue clearing. The hydrogel monomer (HM) solution recipe included 1% (wt/vol) acrylamide, 0.0125-0.05% (wt/vol) bisacrylamide, 4% paraformaldehyde, lx PBS, deionized water, and 0.25% of the thermal initiator VA-044 (Wako Chemicals, NC0632395). A trans-cardial perfusion was performed with HM and the brain was incubated overnight or 2 days at 4° C. with the HM solution. The tissue was then de-gassed to replace the oxygen with nitrogen, followed by incubation at 37° C. for 3-4 hours for hydrogel polymerization. Tissue was cleared in a 37° C. shaking incubator with a buffered clearing solution consisting of 4% (wt/vol) sodium dodecyl sulfate and 0.2 M boric acid (pH 8.5) with solution replacement every one to two days. After this step, the clearing solution was washed off with a 0.2 M boric acid buffer (pH 8.5)/0.1% Triton X-100. For imaging, tissues were mounted in 60% 2, 20-Thiodiethanol (TDE) with 1×PBS.

RNA In Situ Hybridization

Human CNS tissue was fixed in 4% PFA, cryoprotected in 30% sucrose, and samples were serially sectioned onto coverglass slides. RNA in situ hybridization was performed using the RNAscope duplex and chromogenic protocol as suggested by the manufacturer (Advanced Cell Diagnostics). Slides were imaged using a Zeiss Axioimager brightfield camera. A minimum of three different brain samples were subjected to RNA in situ hybridization analyses. Hybridization probes were designed and purchased from RNAscope (Advanced Cell Diagnostics). Patient demographics are provided in FIG. 28. Human tissue was obtained from Johns Hopkins and University of California San Diego Target ALS Autopsy Bank. The use of human tissue and associated decedents' demographic information was approved by the Johns Hopkins University Institutional Review Board and ethics committee (HIPAA Form 5 exemption, Application 11-02-10-01RD) and from the Target ALS Consortium.

In Vivo Imaging and Surgical Procedure

Adult (P60-P90, n=5 mice, 100 astroglia) mice were obtained from crosses of the BAC-GLT1-eGFP and 8.3 kb-tdTomato mice. For repeated in vivo imaging, a cranial window was prepared as previously described. In brief, mice were anesthetized with ketamine xylazine and a craniotomy (3 mm diameter) was placed above the motor cortex. The craniotomy was covered with a permanent glass cover slip (4 mm diameter) and sealed with dental acrylic. For multiphoton imaging, mice were anesthetized with a mixture of oxygen and isoflurane. A metal head-bar was fixed over the craniotomy and placed into a head bar holder for imaging. Mice were imaged weekly up to 500 microns below the dura using the Zeiss LSM 710 at the Johns Hopkins Neuroscience Imaging Core.

Mean Intensity Fluorescence Analysis

8.3-astroglia/Glt1-eGFP mice were stained with Kir4.1/Kcnj10 (see immunohistochemistry) and analyzed with Image-J. Briefly, the entire astroglia and processes were traced with the freehand tool to generate a region of interest (ROI). Next, the ROI was measured for mean pixel density and recorded.

Cortical Thickness Calculation

Motor cortex was imaged from multiple mice (n=5), on the same reference slice, and gross cortical thickness was measured by following the reference brain slice available from the Allen Brain Institute Reference Coronal Atlas.

Sholl Analysis and Dendritic Length

Sholl analysis and dendritic length was determined with Image J as previously described.

Statistical Analysis

Data was plotted in Prism 7 (GraphPad). One-way ANOVA (Bonferroni post-hoc analysis), Two-way Repeated ANOVA, and Student's t-test were used for statistical analyses where appropriate; see figure legends for details. *p<0.05, **p<0.01, ***p<0.001. Standard error of the mean was used for errors bars ±mean.

Multi-Electrode Array Recordings

Mult-electrode arrays were used to make extracellular voltage measurements and analyze functional connectivity. Primary neurons were co-cultured with astrocytes at a density of 800 cells/mm² on multichannel electrode array plates (MultiChannel Systems 60MEA200/30uR-Ti) coated with polyethleneimine (Sigma) and laminin (Sigma). Recordings were conducted beginning at DIV 14 using an MEA2100 system (MultiChannel Systems) on a stage heated to 37° C. Voltage, spike, and burst measurements were made using MC_RACK software and filtered using a second order butterworth filter with a 200 Hz cutoff frequency. Spikes were identified as instantaneous time points of voltages that exceed a threshold of at least five standard deviations from baseline. Spike time stamps were exported using MCS DataManager and further analyzed using MEAnalyzer, where electrodes with high baseline noise (>60 μV) were excluded from analysis. Bursts were identified as clusters of at least four spikes occurring within 100 ms of each other. MEAnalyzer was used to calculate spike rate, burst rate, burst length, the number of spikes in bursts, the percent of spikes organized in bursts, and functional connectivity graphs based on spike rate. For the functional connectivity graphs each electrode was considered a node and an “edge” was created between two nodes if the connection strength between them exceeded a threshold of 0.5. Connection strength was defined as cross-correlation of binned spike rates (0.2 s bin widths) at a time lag of 0 exceeded 0.5. The cross correlation of electrode and at time lag for a recording of length T is as: Once the connectivity graphs were created, several metrics were used to evaluate network properties. Node degree is defined as the total number of connections for each node, represented as a percentage of total nodes, and edge weight is the connection strength between two nodes.

Open Field

Spontaneous locomotion was analyzed using a Versamax Animal Activity Monitoring System with infrared beams (AccuScan Instruments Inc., Columbus, Ohio, USA), In brief, mice were placed in activity chambers for a total duration of 30 minutes. Horizontal and vertical activities as well as the amount of time spent in the center or along the chamber walls were automatically recorded.

Y-Maze

This test evaluates spatial working memory in a Y-maze. Mice were placed in one arm and spontaneous activity was recorded for 5 minutes. The number of entries in each of the three arms as well as the percentage of spontaneous alternations (mouse visiting all three arms without entering the same arm twice) was being recorded.

Materials and Methods for ALS Studies

Animal Models

SOD1 G93A (Jackson Laboratory) and 8.3 kb-tdTomato mice were used for in vivo experiments. The care and treatment of animals were in accordance with the NIH Guide for the Care and Use of Laboratory Animals, the Guidelines for the Use of Animals in Neuroscience Research, and the Johns Hopkins University IACUC. Mice were housed at standard temperature (21° C.) in a light-controlled environment with ad libitum access to food and water. SOD1 G93A mice were crossed with 8.3 kb-tdTomato mice to generate double transgenic mice. Littermates were used as controls and for comparisons between different astroglia populations.

Human iPSC-Astroglia Differentiation

Patient fibroblasts were collected at Johns Hopkins Hospital with patient consent (IRB protocol: NA 00021979) as described⁷. In vitro hiPSC-astroglia were cultured and differentiated as previously described. Briefly, iPSCs were converted into rosettes and then subjected to neural induction toward the neural progenitor cell (NPC) fate via SMAD inhibition. After differentiation into NPCs, treatment with fetal bovine serum was added to induce astrogenesis. Astroglia-differentiating NPCs were subjected to cell passaging once confluency reached 80-90%. All astroglia were analyzed at the mature state (12-13 weeks) and verified with appropriate astroglia markers as previously published. Patient demographics are available in FIG. 28. Cells were differentiated and imaged at age-matched time points.

Immunohistochemistry

Mice were injected with a lethal dose of ketamine xylazine and immediately perfused with 1×PBS followed by 4% paraformaldehyde. Post-perfusion, brain, cerebellum, and spinal cord tissue were collected and cyroprotected in 30% sucrose. Tissue samples were prepared for cryostat sectioning and sectioned at a thickness of 20 μm (brain) or 10 μm (spinal cord). Sections were treated with blocking buffer (5% donkey serum and 0.1% Triton-X 100 in PBS) for 60 min at room temperature. The following primary antibodies were used: LGR6 (Abcam, R&D Systems), KCNJ10 (Alomone Labs). Secondary antibodies were donkey anti-rabbit Alexa Fluor 647 (Abcam), donkey anti-mouse Alexa Fluor 647 (Abcam), donkey anti-rabbit 488 (Abcam), donkey anti-mouse 488 (Abcam), donkey anti-rabbit Cy3 (Abcam), and donkey anti-mouse Cy3 (Abcam). Labeled cells were manually counted or overall fluorescence calculated in Zen Zeiss 2012 software or using spot detection in Bitplane Imaris software 8.3.1. All images were captured using a Zeiss LSM 700 confocal microscope, Zeiss Axioimager, or Zeiss LSM 800 confocal microscope. A minimum of three mice per group were used for each experiment at ages P120.

Immunocytochemistry

Cells were stained as previously described. Briefly, cells were grown until the appropriate time point or confluency followed by fixation in 4% PFA and 0.3% Triton-X-100 permeabilization. Cells were then stained as described above (see Immunohistochemistry).

1. A method of using a norrin protein or functional part thereof for treating or preventing dendritic spine loss in a subject comprising the steps of:
administering to a subject having or at risk of having dendritic spine loss a pharmaceutical composition comprising an agent selected from the group consisting of a salt, solvate, or stereoisomer of a norrin protein or functional part thereof, a vector expressing the norrin protein or functional part thereof, and a combination thereof; and a pharmaceutically acceptable carrier; and
treating or preventing the dendritic spine loss in the subject.

2. The method of claim 1 wherein the vector is selected from the group consisting of nanoparticles, retrovirsuses, adenoviruses or a combination thereof.

3. The method of claim 1 wherein the subject has or is at risk for amyotrophic lateral sclerosis (ALS).

4. The method of claim 1 wherein the norrin protein or functional part thereof is selected from the group consisting of SEQ ID NOs: 1,4,6,7 and a combination thereof.

5. The method of claim 1 wherein the norrin protein or functional part thereof is expressed from a nucleic acid sequence selected from the group consisting of SEQ ID NOs: 2, 3, 5, and a combination thereof.

6. A method of using an agonist of a Leucine Rich Repeat Containing G Protein Coupled Receptor 6 (LGR6) for treating or preventing dendritic spine loss in a subject comprising the steps of:
administering to a subject having or at risk of having dendritic spine loss a pharmaceutical composition comprising an agonist of LGR6 and a pharmaceutically acceptable carrier; and
treating or preventing the dendritic spine loss in the subject.

7. The method of claim 6 wherein the agonist is R-spondin-1 (RSPO1), or a functional part thereof.

8. The method of claim 7 wherein the RSPO1 is selected from the group consisting of SEQ ID NOs: 15, 16, and a combination thereof.

9. The method of claim 6 wherein the agonist of LGR6 is a vector that expresses the agonist of LGR6.

10. The method of claim 9 wherein the LGR6 agonist is RSPO1, or a functional part thereof.

11. The method of claim 9 wherein the vector comprises nucleic acid sequences selected from the group consisting of SEQ ID NOs: 8, 9, 10, 11, 12 and a combination thereof.

12. The method claim 6 further comprising the step of administering a pharmaceutical composition comprising a salt, solvate, or steriosomer of a norrin protein or functional part thereof, and a pharmaceutically acceptable carrier.

13. The method of claim 6 wherein the condition of dendritic spine loss is amyotrophic lateral sclerosis (ALS).

14. A method of using a modulator of the expression of Norrin for treating or preventing a condition of dendritic spine loss in a subject comprising the steps of:
administering to a subject having or at risk of having dendritic spine loss a pharmaceutical composition comprising a modulator of the expression of Norrin and a pharmaceutically acceptable carrier to a subject; and
treating or preventing the condition of dendritic spine loss.

15. The method of claim 14 wherein the modulator is an agonist of LGR6.

16. The method of claim 14 wherein the modulator is RSPO1, or functional part thereof.

17. The method of claim 16 wherein the RSPO1 is selected from the group consisting of SEQ ID NOs: 15, 16, and a combination thereof.

18. The method of claim 14 wherein the modulator is a vector that expresses an agonist of LGR6.

19. The method of 18 wherein the vector comprises nucleic acid sequences selected from the group consisting of SEQ ID NOs: 8, 9, 10, 11, 12 and a combination thereof.

20. The method of claim 18 wherein the agonist is RSPO1, or a functional part thereof.

21. The method of claim 14 wherein the condition of dendritic spine loss is ALS.

22-25. (canceled)


SEQUENCE LISTING (16 sequences)

SEQ ID NO: 1 - protein, 133 aa, Homo sapiens
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 2 - DNA, 2058 bp, Homo sapiens
aagatgctccgtggaagggagccgagcggtgggcagaggctgagtccccgataacgagcgcctcacatttccgtggcattcccatttgctagtgcgctgctgcggccgcacgcctgattgatatatgactgcaatggcacttttccatttgacattctctctctctctctccctctctctctctccctctctctctccctctctctctctccctgtgtcgcttaaacaacagtcctaacttttgtgtgttgcaaatataaaaggcaagccatgtgacagagggacagaagaacaaaagcatttggaagtaacaggacctctttctagctctcagaaaagtctgagaagaaaggagccctgcgttcccctaagctgtgcagcagatactgtgatgatggattgcaagtgcaaagagtaagacaaaactccagcacataaaggacaatgacaaccagaaagcttcagcccgatcctgccctttccttgaacgggactggatcctaggaggtgaagccatttccaattttttgtcctctgcctccctctgctgttcttctagagaagtttttccttacaacaatgagaaaacatgtactagctgcatccttttctatgctctccctgctggtgataatgggagatacagacagtaaaacggacagctcattcataatggactcggaccctcgacgctgcatgaggcaccactatgtggattctatcagtcacccattgtacaagtgtagctcaaagatggtgctcctggccaggtgcgaggggcactgcagccaggcgtcacgctccgagcctttggtgtcgttcagcactgtcctcaagcaacccttccgttcctcctgtcactgctgccggccccagacttccaagctgaaggcactgcggctgcgatgctcagggggcatgcgactcactgccacctaccggtacatcctctcctgtcactgcgaggaatgcaattcctgaggcccgctgctgtgtgtggcttctggatgggacaactgtagaggcagttcgaccagccagggaaagactggcaagaaaagagttaaggcaaaaaaggatgcaacaattctcccgggactctgcatattctagtaataaagactctacatgcttgttgacagagagagatactctgggaacttctttgcagttcccatctcctttctctggtacaatttcttttggttcattttcagattcaggcattttcccccttggctctcaatgctgtttgggtttccaacaattcagcattagtgggaaaaagtgggccctcatacacaagcgtgtcaggctgtcagtgtttggtgcacgctggggaagaatttactttggaaagtagaaaagcccagcttttcctgggacatcttctgttattgttgatgtttttttttaccttgtcattttggtctaaggttgccattgctgctaaaggttaccgatttcaaagtccagataccaagcatgtggatatgtttagctacgtttactcacagccagcgaactgacattaaaataactaacaaacagattcttttatgtgatgctggaactcttgacagctataattattattcagaaatgactttttgaaagtaaaagcagcataaagaatttgtcacaggaaggctgtctcagataaattatggtaaaattttgtaagggagcagacttttaaagacttgcacaaatacggatcctgcactgactctggaaaaggcatatatgtactagtggcatggagaatgcaccatactcatgcatgcaaattagacaaccaagtatgaatctatttgtgggtgtgctatagctttagccgtgtcacgggcatcattctctaatatccacttgtccatgtgaaacatgttgccaaaatggtggcctggcttgtcttctgaacgtttggttcaaatgtgttttggtcctggaggctcaaattttgagttattcccacgttttgaaataaaaagagtatattcaaaaaaaaaaaaaaaaaaaaaaaaaaaaaaaaaaaaaaaaaaaaaaaaaaaaaaaaaa

SEQ ID NO: 3 - DNA, 1962 bp, Mus musculus
ctcatcttaagatgctccatggaaaagatccgagcagtgggcagaggctgtgagtccccgataacgagagcgcctaacatttccgtggtattcccatttgctagagcgctgctgcggccacacgcctgattgatatatgcctgcaatggcacttttccatttgacactctccctctctctctccctctctctccctctctctctctccctctctctccctctctctccctgggtcgcttaaacaacagtcctaacttttgtgtgttgcaaatataaaaggcaagccatgtgacagagggacagaagaacaaaagcatttggaagtaacaggacctctttctagctctcagaaaagtctgagaagaaaggagccctgcgtttcccccaagctgtgcagcacatactgctgtgatcggttgcaagtgaaagaagcaagagaaattcctgcatacaaaggacaatgacaaccagaaagcggcagccctatcctgctctggccttgaaagggactggatcctaggaggtggcagcatttccaatctattgtcctctgtctccctctgctgtttttctggaggagtttccctttacaacaatgagaaatcatgtactagctgcatccatttctatgctctccctgctggccataatgggagatacagacagcaaaacagacagttcatttctgatggactctcaacgctgcatgagacaccattatgtcgattctatcagtcacccactgtacaaatgtagctcaaagatggtgctcctggccagatgtgaggggcactgcagccaggcatcacgctctgagcccttggtgtccttcagcactgtcctcaagcaacctttccgttcctcctgtcactgctgccgaccccagacttccaagctgaaggctctgcgtctgcgctgctcagggggcatgcgacttactgccacttaccggtacatcctctcctgtcactgtgaggaatgcagctcctgagacttgctgatgattggctttctgactggcacaaccacaggagcagttcaacctgccagagacggactggcaagaaaagagttaaggcagataaagatggagcaagtcccataggattttgcatattcttgtcctaaagactcaatgtgcttttgacagaaagtgactctgggaacttgcttttcattcccatctcctttccctggaagaatttcttttggttactttacagattcaggcatttcccctgttggctctaattgtggtttgggtgcctgacagctctgcattagtgggaaaatgtggggccctgtgcaatagcatgtcaggctgttcctatttggtgcatattagggaaaattttacctaactctccttaggagatctttgcttgttgtttcccctggtcattttggtctaagatttgcctctaaagtttcctggtttcaagatctggacacccagtccatggatgtttagtgaggcttactcacagccagctaactgctactaaaataactaacacatgggttcttttatgtgacagcgggactcctgaccactatagtaattattcagaagtgactgaggggatataaatgtggcagaggaatttataatctgaagccttttgtgaggaagcaggctttcacacatacacactcaggtggatcctgcactgactctggagaaggcatacattatacttggtgtggagaacacaccatactcataattgagcattagtcaagcatgtaggaatctacttgtgggtgtgcaatagcttcagccatatcttagctatatccacctgtctatgtgaagcttgttgccgtagtggtggcccgacttattgtctgaaatttttgtttcaatatatttttggtcctcgaagctcaaattttgaagtctccccatgttttcaaataaaaatagagtaccttcaaaaaaaaaaaaaaaaaaaaaaaaaa

SEQ ID NO: 4 - protein, 131 aa, Mus musculus
MRNHVLAASISMLSLLAIMGDTDSKTDSSFLMDSQRCMRHHYVDSISHPLYKCSSKMVLLARCEGHCSQASRSEPLVSFSTVLKQPFRSSCHCCRPQTSKLKALRLRCSGGMRLTATYRYILSCHCEECSS

SEQ ID NO: 5 - DNA, 1076 bp, Mus musculus
  CDS (140)..(532)
  modified_base (887)..(887): a, c, t, g, unknown or other
agcaagcacaggtggcagcggctgcaggggcgcatcgccggcgtgcgccctcctgcagccctgggcgcatcgctctctcggggaagccaccctcggagcccccggagctccccgccaagcgccataagcttcgatcgccatgagaaatcatgtactagctgcatccatttctatgctctccctgctggccataatgggagatacagacagcaaaacagacagttcatttctgatggactctcaacgctgcatgagacaccattatgtcgattctatcagtcacccactgtacaaatgtagctcaaagatggtgctcctggccagatgtgaggggcactgcagccaggcatcacgctctgagcccttggtgtccttcagcactgtcctcaagcaacctttccgttcctcctgtcactgctgccgaccccagacttccaagctgaaggctctgcgtctgcgctgctcagggggcatgcgacttactgccacttaccggtacatcctctcctgtcactgtgaggaatgcagctcctgaacgcgtacgcggccgctcgagagcgctaagctccgagggccgccaccacctgttcctgtacggcatggacgagctgtacaagtaagaattcgatatcggatccgcagatcccggccagataccgatgctgccgcagcaaaagcaggagcagatgccgccgtcgcaggcgaagatgtcgcagacggaggaggcgatgctgccggcggaggaggcgaagtaagtagagggctgggctgggctgtggggggtgtggggtgcgggactgggcagtctgggagtccctctcaccacttttcttacctttctaggatgctgccgtcgccgccgctcatacaccataaggtgtaaaaantactagatgcacagaatagcaagtccatcaaaactcctgcgtgagaattttaccagacttcaagagcatctcgccacatcttgaaaaatgccaccgtccgatgaaaaacaggagcctgctaaggaacaatgccacctgtcaataaatgttgaaaactcatcccattcctgcctcttggtccttgggctt

SEQ ID NO: 6 - protein, 133 aa, Homo sapiens; identical to SEQ ID NO: 1

SEQ ID NO: 7 - protein, 131 aa, Mus musculus; identical to SEQ ID NO: 4

SEQ ID NO: 8 - DNA, 3089 bp, Homo sapiens
attccctccctggtgctcgcagaggactggcccctctccgggctgggagctccggccgagcggaggcgcgacggagagcaccagcgcagggcagagagcccggagcgaccggccagagtagggcatccgctcgggtgctgcggagaacgagggcagctccgagccgccccggaggaccgatgcgccgggtggggcgctggccccgagggcgtgagccgtccgcagattgagcaacttgggaacgggcgggcggagcgcaggcgagccgggcgcccaggacagtcccgcagcgggcgggtgagcgggccgcgccctcgcccctcccgggcctgcccccgtcgcgactggcagcacgaagctgagattgtggtttcctggtgattcaggtgggagtgggccagaagatcaccgctggcaaggactggtgtttgtcaactgtaaggactcatggaacagatctaccagggattctcagaccttagtttgagaaatgctgcaattaaaggcaaatcctatcactctgagtgatcgctttggtgtcgaggcaatcaaccataaagataaatgcaaatatggaaattgcataacagtactcagtattaaggttggtttttggagtagtccctgctgacgtgacaaaaagatctctcatatgatattccgaggtatctttgaggaagtctctctttgaggacctccctttgagctgatggagaactgggctccccacaccctctctgtccccagctgagattatggtggatttgggctacggcccaggcctgggcctcctgctgctgacccagccccagaggtgttagcaagagccgtgtgctatccaccctccccgagaccacccctccgaccaggggcctggagctggcgcgtgactatgcggcttgggctgtgtgtggtggccctggttctgagctggacgcacctcaccatcagcagccgggggatcaaggggaaaaggcagaggcggatcagtgccgaggggagccaggcctgtgccaaaggctgtgagctctgctctgaagtcaacggctgcctcaagtgctcacccaagctgttcatcctgctggagaggaacgacatccgccaggtgggcgtctgcttgccgtcctgcccacctggatacttcgacgcccgcaaccccgacatgaacaagtgcatcaaatgcaagatcgagcactgtgaggcctgcttcagccataacttctgcaccaagtgtaaggagggcttgtacctgcacaagggccgctgctatccagcttgtcccgagggctcctcagctgccaatggcaccatggagtgcagtagtcctgcgcaatgtgaaatgagcgagtggtctccgtgggggccctgctccaagaagcagcagctctgtggtttccggaggggctccgaggagcggacacgcagggtgctacatgcccctgtgggggaccatgctgcctgctctgacaccaaggagacccggaggtgcacagtgaggagagtgccgtgtcctgaggggcagaagaggaggaagggaggccagggccggcgggagaatgccaacaggaacctggccaggaaggagagcaaggaggcgggtgctggctctcgaagacgcaaggggcagcaacagcagcagcagcaagggacagtggggccactcacatctgcagggcctgcctagggacactgtccagcctccaggcccatgcagaaagagttcagtgctactctgcgtgattcaagctttcctgaactggaacgtcgggggcaaagcatacacacacactccaatccatccatgcatacatagacacaagacacacacgctcaaacccctgtccacatatacaaccatacatacttgcacatgtgtgttcatgtacacacgcagacacagacaccacacacacacatacacacacacacacacacacacctgaggccaccagaagacacttccatccctcgggcccagcagtacacacttggtttccagagctcccagtggacatgtcagagacaacacttcccagcatctgagaccaaactgcagaggggagccttctggagaagctgctgggatcggaccagccactgtggcagatgggagccaagcttgaggactgctggtgacctgggaagaaaccttcttcccatcctgttcagcactcccagctgtgtgactttatcgttggagagtattgttacccttccaggatacatatcagggttaacctgactttgaaaactgcttaaaggtttatttcaaattaaaacaaaaaaatcaacgacagcagtagacacaggcaccacattcctttgcagggtgtgagggtttggcgaggtatgcgtaggagcaagaagggacagggaatttcaagagaccccaaatagcctgctcagtagagggtcatgcagacaaggaagaaaacttaggggctgctctgacggtggtaaacaggctgtctatatccttgttactcagagcatggcccggcagcagtgttgtcacagggcagcttgttaggaatgagaatctcaggtctcattccagacctggtgagccagagtctaaattttaagattcctgatgattggcatgttacccaaatttgagaagtgctgctgtaattccccttaaaggacgggagaaagggccccggccatcttgcagcaggagggattctggtcagctataaaggaggactttccatctgggagaggcagaatctatatactgaagggctagtggcactgccaggggaagggagtgcgtaggcttccagtgatggttggggacaatcctgcccaaaggcagggcagtggatggaataactccttgtggcattctgaagtgtgtgccaggctctggactaggtgctaggtttccagggaggagccaaacacgggccttgctcttgtggagcttagaggttggtggggaagaaaataggcatgcaccaaggaattgtacaaacacatatataactacaaaaggatggtgccaagggcaggtgaccactggcatctatgcttagctatgaaagtgaataaagcagaataaaaataaaatactttctctcagg

SEQ ID NO: 9 - DNA, 2910 bp, Homo sapiens
attccctccctggtgctcgcagaggactggcccctctccgggctgggagctccggccgagcggaggcgcgacggagagcaccagcgcagggcagagagcccggagcgaccggccagagtagggcatccgctcgggtgctgcggagaacgagggcagctccgagccgccccggaggaccgatgcgccgggtggggcgctggccccgagggcgtgagccgtccgcagattgagcaacttgggaacgggcgggcggagcgcaggcgagccgggcgcccaggacagtcccgcagcgggcgggtgagcgggccgcgccctcgcccctcccgggcctgcccccgtcgcgactggcagcacgaagctgagattgtggtttcctggtgattcaggtgggagtgggccagaagatcaccgctggcaaggactgggttggtttttggagtagtccctgctgacgtgacaaaaagatctctcatatgatattccgaggtatctttgaggaagtctctctttgaggacctccctttgagctgatggagaactgggctccccacaccctctctgtccccagctgagattatggtggatttgggctacggcccaggcctgggcctcctgctgctgacccagccccagaggtgttagcaagagccgtgtgctatccaccctccccgagaccacccctccgaccaggggcctggagctggcgcgtgactatgcggcttgggctgtgtgtggtggccctggttctgagctggacgcacctcaccatcagcagccgggggatcaaggggaaaaggcagaggcggatcagtgccgaggggagccaggcctgtgccaaaggctgtgagctctgctctgaagtcaacggctgcctcaagtgctcacccaagctgttcatcctgctggagaggaacgacatccgccaggtgggcgtctgcttgccgtcctgcccacctggatacttcgacgcccgcaaccccgacatgaacaagtgcatcaaatgcaagatcgagcactgtgaggcctgcttcagccataacttctgcaccaagtgtaaggagggcttgtacctgcacaagggccgctgctatccagcttgtcccgagggctcctcagctgccaatggcaccatggagtgcagtagtcctgcgcaatgtgaaatgagcgagtggtctccgtgggggccctgctccaagaagcagcagctctgtggtttccggaggggctccgaggagcggacacgcagggtgctacatgcccctgtgggggaccatgctgcctgctctgacaccaaggagacccggaggtgcacagtgaggagagtgccgtgtcctgaggggcagaagaggaggaagggaggccagggccggcgggagaatgccaacaggaacctggccaggaaggagagcaaggaggcgggtgctggctctcgaagacgcaaggggcagcaacagcagcagcagcaagggacagtggggccactcacatctgcagggcctgcctagggacactgtccagcctccaggcccatgcagaaagagttcagtgctactctgcgtgattcaagctttcctgaactggaacgtcgggggcaaagcatacacacacactccaatccatccatgcatacatagacacaagacacacacgctcaaacccctgtccacatatacaaccatacatacttgcacatgtgtgttcatgtacacacgcagacacagacaccacacacacacatacacacacacacacacacacacctgaggccaccagaagacacttccatccctcgggcccagcagtacacacttggtttccagagctcccagtggacatgtcagagacaacacttcccagcatctgagaccaaactgcagaggggagccttctggagaagctgctgggatcggaccagccactgtggcagatgggagccaagcttgaggactgctggtgacctgggaagaaaccttcttcccatcctgttcagcactcccagctgtgtgactttatcgttggagagtattgttacccttccaggatacatatcagggttaacctgactttgaaaactgcttaaaggtttatttcaaattaaaacaaaaaaatcaacgacagcagtagacacaggcaccacattcctttgcagggtgtgagggtttggcgaggtatgcgtaggagcaagaagggacagggaatttcaagagaccccaaatagcctgctcagtagagggtcatgcagacaaggaagaaaacttaggggctgctctgacggtggtaaacaggctgtctatatccttgttactcagagcatggcccggcagcagtgttgtcacagggcagcttgttaggaatgagaatctcaggtctcattccagacctggtgagccagagtctaaattttaagattcctgatgattggcatgttacccaaatttgagaagtgctgctgtaattccccttaaaggacgggagaaagggccccggccatcttgcagcaggagggattctggtcagctataaaggaggactttccatctgggagaggcagaatctatatactgaagggctagtggcactgccaggggaagggagtgcgtaggcttccagtgatggttggggacaatcctgcccaaaggcagggcagtggatggaataactccttgtggcattctgaagtgtgtgccaggctctggactaggtgctaggtttccagggaggagccaaacacgggccttgctcttgtggagcttagaggttggtggggaagaaaataggcatgcaccaaggaattgtacaaacacatatataactacaaaaggatggtgccaagggcaggtgaccactggcatctatgcttagctatgaaagtgaataaagcagaataaaaataaaatactttctctcagg

SEQ ID NO: 10 - DNA, 2589 bp, Homo sapiens
attccctccctggtgctcgcagaggactggcccctctccgggctgggagctccggccgagcggaggcgcgacggagagcaccagcgcagggcagagagcccggagcgaccggccagagtagggcatccgctcgggtgctgcggagaacgagggcagctccgagccgccccggaggaccgatgcgccgggtggggcgctggccccgagggcgtgagccgtccgcagattgagcaacttgggaacgggcgggcggagcgcaggcgagccgggcgcccaggacagtcccgcagcgggcgggtgagcgggccgcgccctcgcccctcccgggcctgcccccgtcgcgactggcagcacgaagctgagattgtggtttcctggtgattcaggtgggagtgggccagaagatcaccgctggcaaggactgggttggtttttggagtagtccctgctgacgtgacaaaaagatctctcatatgatattccgagtcagtgccgaggggagccaggcctgtgccaaaggctgtgagctctgctctgaagtcaacggctgcctcaagtgctcacccaagctgttcatcctgctggagaggaacgacatccgccaggtgggcgtctgcttgccgtcctgcccacctggatacttcgacgcccgcaaccccgacatgaacaagtgcatcaaatgcaagatcgagcactgtgaggcctgcttcagccataacttctgcaccaagtgtaaggagggcttgtacctgcacaagggccgctgctatccagcttgtcccgagggctcctcagctgccaatggcaccatggagtgcagtagtcctgcgcaatgtgaaatgagcgagtggtctccgtgggggccctgctccaagaagcagcagctctgtggtttccggaggggctccgaggagcggacacgcagggtgctacatgcccctgtgggggaccatgctgcctgctctgacaccaaggagacccggaggtgcacagtgaggagagtgccgtgtcctgaggggcagaagaggaggaagggaggccagggccggcgggagaatgccaacaggaacctggccaggaaggagagcaaggaggcgggtgctggctctcgaagacgcaaggggcagcaacagcagcagcagcaagggacagtggggccactcacatctgcagggcctgcctagggacactgtccagcctccaggcccatgcagaaagagttcagtgctactctgcgtgattcaagctttcctgaactggaacgtcgggggcaaagcatacacacacactccaatccatccatgcatacatagacacaagacacacacgctcaaacccctgtccacatatacaaccatacatacttgcacatgtgtgttcatgtacacacgcagacacagacaccacacacacacatacacacacacacacacacacacctgaggccaccagaagacacttccatccctcgggcccagcagtacacacttggtttccagagctcccagtggacatgtcagagacaacacttcccagcatctgagaccaaactgcagaggggagccttctggagaagctgctgggatcggaccagccactgtggcagatgggagccaagcttgaggactgctggtgacctgggaagaaaccttcttcccatcctgttcagcactcccagctgtgtgactttatcgttggagagtattgttacccttccaggatacatatcagggttaacctgactttgaaaactgcttaaaggtttatttcaaattaaaacaaaaaaatcaacgacagcagtagacacaggcaccacattcctttgcagggtgtgagggtttggcgaggtatgcgtaggagcaagaagggacagggaatttcaagagaccccaaatagcctgctcagtagagggtcatgcagacaaggaagaaaacttaggggctgctctgacggtggtaaacaggctgtctatatccttgttactcagagcatggcccggcagcagtgttgtcacagggcagcttgttaggaatgagaatctcaggtctcattccagacctggtgagccagagtctaaattttaagattcctgatgattggcatgttacccaaatttgagaagtgctgctgtaattccccttaaaggacgggagaaagggccccggccatcttgcagcaggagggattctggtcagctataaaggaggactttccatctgggagaggcagaatctatatactgaagggctagtggcactgccaggggaagggagtgcgtaggcttccagtgatggttggggacaatcctgcccaaaggcagggcagtggatggaataactccttgtggcattctgaagtgtgtgccaggctctggactaggtgctaggtttccagggaggagccaaacacgggccttgctcttgtggagcttagaggttggtggggaagaaaataggcatgcaccaaggaattgtacaaacacatatataactacaaaaggatggtgccaagggcaggtgaccactggcatctatgcttagctatgaaagtgaataaagcagaataaaaataaaatactttctctcagg

SEQ ID NO: 11 - DNA, 2721 bp, Homo sapiens
attccctccctggtgctcgcagaggactggcccctctccgggctgggagctccggccgagcggaggcgcgacggagagcaccagcgcagggcagagagcccggagcgaccggccagagtagggcatccgctcgggtgctgcggagaacgagggcagctccgagccgccccggaggaccgatgcgccgggtggggcgctggccccgagggcgtgagccgtccgcagattgagcaacttgggaacgggcgggcggagcgcaggcgagccgggcgcccaggacagtcccgcagcgggcgggtgagcgggccgcgccctcgcccctcccgggcctgcccccgtcgcgactggcagcacgaagctgagattgtggtttcctggtgattcaggtgggagtgggccagaagatcaccgctggcaaggactgggttggtttttggagtagtccctgctgacgtgacaaaaagatctctcatatgatattccgaggtatctttgaggaagtctctctttgaggacctccctttgagctgatggagaactgggctccccacaccctctctgtccccagctgagattatggtggatttgggctacggcccaggcctgggcctcctgctgctgacccagccccagaggtgttagcaagagccgtgtgctatccaccctccccgagaccacccctccgaccaggggcctggagctggcgcgtgactatgcggcttgggctgtgtgtggtggccctggttctgagctggacgcacctcaccatcagcagccgggggatcaaggggaaaaggcagaggcggatcagtgccgaggggagccaggcctgtgccaaaggctgtgagctctgctctgaagtcaacggctgcctcaagtgctcacccaagctgttcatcctgctggagaggaacgacatccgccaggtgggcgtctgcttgccgtcctgcccacctggatacttcgacgcccgcaaccccgacatgaacaagtgcatcaaatgcaagatcgagcactgtgaggcctgcttcagccataacttctgcaccaagtgtaaggagggcttgtacctgcacaagggccgctgctatccagcttgtcccgagggctcctcagctgccaatggcaccatggagtgcagtagtcctgggcagaagaggaggaagggaggccagggccggcgggagaatgccaacaggaacctggccaggaaggagagcaaggaggcgggtgctggctctcgaagacgcaaggggcagcaacagcagcagcagcaagggacagtggggccactcacatctgcagggcctgcctagggacactgtccagcctccaggcccatgcagaaagagttcagtgctactctgcgtgattcaagctttcctgaactggaacgtcgggggcaaagcatacacacacactccaatccatccatgcatacatagacacaagacacacacgctcaaacccctgtccacatatacaaccatacatacttgcacatgtgtgttcatgtacacacgcagacacagacaccacacacacacatacacacacacacacacacacacctgaggccaccagaagacacttccatccctcgggcccagcagtacacacttggtttccagagctcccagtggacatgtcagagacaacacttcccagcatctgagaccaaactgcagaggggagccttctggagaagctgctgggatcggaccagccactgtggcagatgggagccaagcttgaggactgctggtgacctgggaagaaaccttcttcccatcctgttcagcactcccagctgtgtgactttatcgttggagagtattgttacccttccaggatacatatcagggttaacctgactttgaaaactgcttaaaggtttatttcaaattaaaacaaaaaaatcaacgacagcagtagacacaggcaccacattcctttgcagggtgtgagggtttggcgaggtatgcgtaggagcaagaagggacagggaatttcaagagaccccaaatagcctgctcagtagagggtcatgcagacaaggaagaaaacttaggggctgctctgacggtggtaaacaggctgtctatatccttgttactcagagcatggcccggcagcagtgttgtcacagggcagcttgttaggaatgagaatctcaggtctcattccagacctggtgagccagagtctaaattttaagattcctgatgattggcatgttacccaaatttgagaagtgctgctgtaattccccttaaaggacgggagaaagggccccggccatcttgcagcaggagggattctggtcagctataaaggaggactttccatctgggagaggcagaatctatatactgaagggctagtggcactgccaggggaagggagtgcgtaggcttccagtgatggttggggacaatcctgcccaaaggcagggcagtggatggaataactccttgtggcattctgaagtgtgtgccaggctctggactaggtgctaggtttccagggaggagccaaacacgggccttgctcttgtggagcttagaggttggtggggaagaaaataggcatgcaccaaggaattgtacaaacacatatataactacaaaaggatggtgccaagggcaggtgaccactggcatctatgcttagctatgaaagtgaataaagcagaataaaaataaaatactttctctcagg

SEQ ID NO: 12 - DNA, 1834 bp, Mus musculus
ggattccctccctcgtgcgagccggggaccggcccctctccgggcgcggggcgcagagcccgggcggcgcactgcggggcccgcgcgggccgccccagcaccaatgcaccgggcggggcgctggcggccgagaaggcattgagcaactgggcggcgggcggagcgcggggccgacggcaacgcgggacccagtggccgcgccctcgcccctccgggctgccccgccacggccgctgcgccaggtctatcttgggggtggttctctgctggcgtgagaagacttctcatgtgaccctctgaggtggattcaagcaggacaggacctccctttggaccaatggagaagccggctccaaaccctctcggatcccagctaaggttatggtggatccgggcctggctctcctgccactgacccagcctcagagccttttagcaagagaccacccctcctgccaggggcccggggctggccagtgactatgcggcttgggctgtgcgtggtggccctggttctgagctggacacacatcgccgtgggcagccgggggatcaagggcaagagacagaggcggatcagtgctgaggggagccaagcctgcgccaagggctgtgagctctgttcagaagtcaacggttgcctcaagtgctcgcccaagctcttcattctgctggagaggaacgacatccgccaggtgggcgtctgcctgccgtcctgcccacctggatactttgatgcccgcaaccccgacatgaacaaatgcatcaaatgcaagatcgagcactgtgaggcctgcttcagccacaacttctgcaccaagtgtcaggagggcttgtacttacacaagggccgctgctatccagcctgccctgagggctctacagccgctaacagcaccatggagtgcggcagtcctgcacaatgtgaaatgagcgagtggtccccgtggggaccctgctccaagaagaggaagctgtgcggtttccggaagggatcggaagagcggacacgcagagtgctccatgctcccgggggagaccacaccacctgctccgacaccaaagagacccgcaagtgtaccgtgcgcaggacgccctgcccagaggggcagaagaggaggaaggggggccagggccggagggagaatgccaacaggcatccggccaggaagaacagcaaggagccgggctccaactctcggagacacaaagggcaacagcagccacagccagggacaacagggccactcacatcagtaggacctacctgggcacagtgaccggtctccagatacctgtggaagagtacagtgctgtactgtataatgagaactttccagaactggagcatctgggagagtccacacataccccatccacccacccatccaactatccatccatccatccatgcacacatatggccacatctgaaaacgtcaacacacacacacacacacacacacacacacacacacattcttgaggtcactgaagacacttctattctgtggcccagctgtatattcagtctttaatgctcttggaagacatatctgagagaacctttcccagcatctgaaactaaggagtggaaccttctggaggaacttctgggacagcatctgacagatggatggcagattggagccaaagctggagcagctgccgagagggagagagagggaaagcgctttcccggcttgagaggcactcccagctgtgagacttgattgtcggagatgagaattattacacatccgtggtacacgtcacggatgacctgacttggaaactgcttaaaggtttatttcaaattaaaaaagagaaaaac

SEQ ID NO: 13 - protein, 118 aa, Artificial Sequence
  Description of Artificial Sequence Synthetic polypeptide
MVLLARCEGHCSQASRSEPLVSFSTVLKQPFRSSCHCCRPQTSKLKALRLRCSGGMRLTATYRYILSCHCEECNSGPLLCVASGWDNCRGSSTSQGKTGKKRVKAKKDATILPGLCIF

SEQ ID NO: 14 - protein, 38 aa, Artificial Sequence
  Description of Artificial Sequence Synthetic polypeptide
MLRGHATHCHLPVHPLLSLRGMQFLRPAAVCGFWMGQL

SEQ ID NO: 15 - protein, 263 aa, Homo sapiens
MRLGLCVVALVLSWTHLTISSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 16 - protein, 265 aa, Mus musculus
MRLGLCVVALVLSWTHIAVGSRGIKGKRQRRISAEGSQACAKGCELCSEVNGCLKCSPKLFILLERNDIRQVGVCLPSCPPGYFDARNPDMNKCIKCKIEHCEACFSHNFCTKCQEGLYLHKGRCYPACPEGSTAANSTMECGSPAQCEMSEWSPWGPCSKKRKLCGFRKGSEERTRRVLHAPGGDHTTCSDTKETRKCTVRRTPCPEGQKRRKGGQGRRENANRHPARKNSKEPGSNSRRHKGQQQPQPGTTGPLTSVGPTWAQ